(19) United States
(12) Patent Application Publication
(10) Pub. No.: US 2002/0132021 A1
(43) Pub. Date: Sep. 19, 2002

(54) Elicited plant products

(76) Inventors: Ilya Raskin, Manalapan, NJ (US); Alexander Poulev, Highland Park, NJ (US)

Correspondence Address: MARSHALL, GERSTEIN & BORUN, 6300 SEARS TOWER, 233 SOUTH WACKER, CHICAGO, IL, 60606-6357, US

(21) Appl. No.: 09/929,328

(22) Filed: Aug. 13, 2001

Related U.S. Application Data
(63) Continuation-in-part of application No. 09/130,185, filed on Aug. 6, 1998, now abandoned.
(63) Continuation-in-part of application No. 09/203,772, filed on Jun. 23, 1998, now abandoned.
Application No. 09/203,772 is a continuation-in-part of application No. 09/067,836, filed on Apr. 28, 1998, now abandoned.
(60) Provisional application No. 60/045,220, filed on Apr. 30, 1997.
(60) Provisional application No. 60/050,441, filed on Jun. 27, 1997.

(51) Int. Cl. A61K 35/78

(52) U.S. Cl. 424/773

(57) ABSTRACT

Intact living plants or plant parts are contacted with water to extract from the plant or plant part exuded chemical compounds, with the extracted chemical compounds subsequently being recovered from the water. The plant is treated with an elicitor or inducer to initiate or increase production of a chemical compound. The roots may be harvested for recovery of the chemical compounds. Valuable substances exuded from or onto a plant surface, such as a plant cuticle or the root of a plant, can be identified as biologically active. Libraries of substances exuded or secreted from various plant species can be elicited or induced to produce one or more of such substances.

This application claims priority to U.S. patent. application Ser. No. 09/130,185, filed Aug. 6, 1998, and U.S. patent application Ser. No. 09/203,772, filed Jun. 23, 1998, which is a continuation-in-part application of U.S. patent application Ser. No. 09/067,836, filed Apr. 28, 1998, which claims priority to U.S. Provisional Application Nos. 60/045,220 and 60/050,441, filed on Apr. 30, 1997 and Jun. 27, 1997, respectively. Each of the above-mentioned provisional and nonprovisional U.S. patent applications are herein expressly incorporated by reference.

### BACKGROUND OF THE INVENTION

Plants have long been recognized as providing a potential source of chemical compounds or more commonly products, known as phytochemicals. A wide variety of compounds of commercial interest, including those having pharmaceutical or therapeutic activity, have been discovered to be plant products. In general, such compounds have been recovered either as a crude extract or as purified compounds, which requires the use of complex extraction and purification procedures. While many therapeutically active compounds have been isolated from extracts of plant roots, methods of screening the plants or plant parts for therapeutically active compounds and isolation of compounds demonstrating such activity from exudates have not been fully investigated.

One source of the chemical compounds or products are the leaf surfaces of the plant. The leaf surfaces of higher plants, in particular, are covered with non-cellular cuticular materials, which are non-living tissue that is heterogeneous in chemical composition. These materials can include lipids, wax, and cutin (biopolymer composing fatty and hydroxy fatty acids), as well as proteins and many secondary metabolites associated with leaf surface or present in hairs or trichomes covering leaf surfaces. These cuticular compounds can be assessed or removed by rapid immersion of intact leaves in organic solvents or by running the solvents over the leaf surface (Martin and Batt, 1958; Purdy and Truter, 1963; S. Fernandes et al., 1964). Generally these techniques can avoid contamination by substances within the leaf making the process, which is significantly different from a total tissue extraction. The amounts of cuticular compounds present on the leaf surfaces of different species are variable, but normally lie in the range 0.01-0.5 mg/cm². Cuticular compounds are usually more frequently obtained from the lower leaf surface rather than the upper leaf surface. Waxes and other cuticular compounds typically are deposited in early stage of leaf growth and continue to deposit throughout the period of leaf expansion (Richmond and Martin, 1959; Baker et al., 1963; Baker et al., 1968). In additional leaf waxes are known to inhibit spore germination of pathogenic fungi (Hafiz, 1952; Topps and Wain, 1957; van Velson 1957; Komo, Akitsma, 1960).

In addition to leaves, plant roots also continuously produce and secrete a characteristically unique set of compounds into their immediate environment (rhizosphere). It has been suggested that up to 10% of photosynthetically fixed carbon is secreted from the roots in the form of biologically active compounds (Shepherd et al 1993; Johansson et al. 1993). Despite the interest and investigation of these such phenomenon, the systematic study of compounds present in root exudates of diverse plant species and their therapeutic activity has not been undertaken. In addition, certain compounds present in root exudates have been shown to play an important role in several biological processes including activation of the Rhizobium genes responsible for the nodulation process (Peters et al 1986; Peters et al. 1988) and, possibly, for vesicular-arbuscular mycorrhiza (VAM) colonization (Tsai and Phillips 1991). Strigol, a germination stimulant for the parasitic plant Striga asitica, has been found in the root exudates of many cereals (Siame et al 1993). Moreover, root-secreted compounds called phytosiderophores may be involved in the acquisition of essential plant nutrients from soils (Cakmak et al 1994) and in defense against such toxic metals as aluminum (de la Fuente et al 1997).

For example, genistein and daidzein are isoflavonoids present in a number of plants which have been recognized as having anti-cancer activity. However, such chemicals currently are available only to the consumer in the form of soybean flours and other crude soybean products. Compounds have been recovered from roots and leafs and used either as a crude root exudate extract or as purified compounds which require the use of complex extraction and purification procedures. Other plant chemicals are available in purified forms; however, they are generally recovered only after a costly and laborious tissue extraction procedure. Accordingly, although there is some information about plant or plant parts producing compounds having therapeutic activity, very little literature, if any, is available regarding the systematic application of chemicals or agents to induce or improve the production of therapeutic compounds, compositions, extracts or exudates in plants. More particularly, there remains a need for a systematic method of inducing or improving the production of chemical compounds or compositions demonstrating antibacterial, antifungal, and anticancer activity.

In particular, such methods do not identify novel agents having therapeutic activity or provide methods of reproducing compounds, extracts, or compositions having therapeutic activity in a consistent manner.

### SUMMARY OF THE INVENTION

The present invention is directed to a method of eliciting chemical compounds in plants. Although a wide variety of elicitors is disclosed, the chemical compound acetic acid is particularly contemplated for inducing or improving compounds or compositions demonstrating anti-microbial (i.e., anti-bacterial or anti-fungal) and/or anti-cancer activity. The plants typically are subjected to treatment and/or conditions to induce (i.e., to initiate or increase) production of such compounds in the plant. Such treatment includes maintaining a living plant or plant portion in contact with water, while alive, in order to induce or improve production of one or more compounds demonstrating therapeutic activity. The living plant or plant portion is subjected to treatment and/or conditions to induce and/or increase production of one or more compounds in the roots of the plant in particular. Such compounds are recovered from the water for example by the process of extracting or exudation. The water may or may not include other ingredients, such as nutrients or elicitors.

The process of the present invention may be employed for commercial production of desired compounds. The commercial production of such compounds can be accomplished in an aqueous medium containing an elicitor. The elicitor can be any elicitor, including for example, chemical elicitors such as acetic acid, which has demonstrated promising activity in inducing and improving compounds having therapeutic activity.

Roots of the plant can be harvested before extraction. The compounds produced or increased in quantity as a result of the treatment can be recovered from the harvested roots and then screened for potential therapeutic activity. The plant and plant parts can be screened for potential compounds of interest by recovering and isolating such compounds from solvent extract of leaves or roots of the plant.

The plants or plant parts of the invention can be specifically grown or maintained for the purpose of recovering compounds therefrom. A chemical compound library generated from the recovered compounds may be used for screening for a desired compound or activity. In this respect, plants or plant parts which are specifically grown or maintained for the purpose of recovering compounds therefrom are contacted with water while alive in order to recover a variety of compounds, particularly for potential screening. The plants or plant parts are preferably contacted with an elicitor to increase the amount and/or diversity of compounds which can be recovered in the root exudate. The chemical compound library may be used for screening for a desired compound or activity.

In a preferred embodiment, the plant is grown hydroponically or aeroponically. In a particularly preferred embodiment, the roots are harvested in a manner such that the plant remains alive and can grow new roots for future harvesting and recovery of additional compounds.

The invention also provides a method of identifying an agent exuded from or onto a plant surface having therapeutic activity. The method comprises: (a) removing cuticular material located on the surface of a leaf of the plant, for example by contacting the leaf surface with a solvent, thereby resulting in a solvent solution; (b) assaying the solvent solution for the identification of agents of the cuticular material which have therapeutic activity; and (c) analyzing the solvent solution so as to identify the agent which has the therapeutic activity.

The plants which are used in the invention may be any one of a wide variety of plants and may be sexually or vegetatively propagated plants as is further described herein. In particular, plants suitable for use in the invention, such as use in the method for eliciting a compound having therapeutic activity as described below, include: Atropa belladonna, Erythrina flabelliformis, Ipomoea tricolor, Erythrina crista, Celosia cristata, Gallium spurium, Laurus nobilis, Vitis labrusca, Gratiola officinalis, Symphitum officinalis, Hosta fortunei, Cassia hebecarpa, Thalictrum flavum, Scutellaria altissima, Portulacca oleracea, Scutellaria certicola, Physalis sp., Geum fauriei, Gentiana tibetica, Linum hirsutum, Aconitum napellus, Podophyllum emodii, Thymus cretaceus, Carlina acaulis, Chamaecrista fasciculata, Pinus pinea, Peganum harmala, Tamarindus indica, Carica papaya, Cistus incanus, Capparis spinosa, Cupressus lusitanica, Diospyros kaki, Eryngium campestre, Aesculus woerlitzensis, Aesculus hippocastanum, Cupressus sempervirens, Celtis occidentalis, Polygonum cuspidatum, Elaeagnus angustifolia, Elaeagnus commutata, Gentiana macrophylla, Brassica rapa, Sesbania exaltata, Sesbania speciosa, Spartina potentiflora, Brassica juncea, Helianthus annuus, Poinsettia sp., Pelargonium zonale, Synapsis sp., Leontopodium alpinum, Lupinus luteus, Buxus microphylla var. japonica, Liatris spicata, Primula japonica, Betula nigra, Filipendula vulgrais, Lobelia siphilitica, Grevillea robusta, Reseda luteola, Gentiana littoralia, Campanula carpatica, Ageratum conizoides, Psidium guajava, Ailanthus altissima, Hydrocotyle asiatica, Brugmansia suaveolens, Thymus pulegioides, Thymus lema-barona, Thymus serphyllum (wild), Gaultheria procumbens, Thymus camosus, Thymus thracicus, Calycanthus floridus, Zingiber officinalis, Lamium dulcis, Thymus praecox “arcticus”, Thymus speciosa, Thymus pseudolamginosus, Thymus vulgraris, Ficus religiosa, Forsythia suspensa, Chelidonium majus, Thymus wooly, Thymus portugalense, Nicotiana tabacum, Thymus cytriodorus “aureus”, Cactus officinailis, Lablab purpurea, Juglans regia, Actinidia chinensis, Hemerocallis sp., Betula pendula, Gardenia jasminoides, Taxodium distichum, Magnolia loebherii, Crataegus praegophyrum, Larix decidua, Thuja orientalis, Thuja ocidentalis, Cupressocyparis leylandii, Pseudotsuga menziesii, Abies firma, Parthenocissus quinquefolia, Allium cemuum, Juniperus “blue pacific”, Taraxacum officinalis, Yucca sp., Tsuga canadensis, Ilex aquifolium, Ilex comuta, Taxus hiksii, Taxus media, Metasequoia glyptostroboides, Pinus bungiana, Buxus sempervirens, Stewartia koreana, Prunus sp., Betula dahurica, Plantago minor, Acer palmatum, Acer campestre, Cotinus coggygria, Quercus robur, Acer truncatum, Achyranthes bidentata, Allium japonicum, Carum capsicum, Agastache mexicana, Prunella vulgaris, Tagetes minuta, Nepeta cataria, Ratibida columnaris, Aster novae-angliae, Myrica cerifera, Pittosporum tobira, Plantago major, Pinus sylvestris, Acorus canadensis, Pieris japonica, Pinus strobus, Trifolium pratense, Prunus serotina, Datura stramonium, Geranium maculatum, Hydrocotyle asiatica, Astragalus sinicus, Centaurea maculata, Ruschia indurata, Myrthus communis, Platanus occidentalis, Licium barbatum, Lavandula officinalis, Grevillea robusta, Hypophaë rhamnoides, Filipendula ulmaria, Betula pendula, Polygonum odoratum, Brugmansia graveolens, Rhus toxicodenta, Armoracia rusticana, Ficus benjaminii, Sufflera sp, Baikiaea recurvata, Asimina triloba, Lippia dulcis, Epilobium augustifolium, Brugmansia suaveolens, Xanthosoma sagittifolium, Monstera deliciosa., Aglaonema commutatus, Dieffenbachia leopoldii, Anthurium andreanum, Syngonium podophyllum, Dracaena fragrans, Ananas comosus, Strelitzia reginae, Dieffenbachia segiune, Syngonium auritum, Dracaena sp., Haemanthus katharinae, Anthurium altersianum, Spathiphyllum grandiflorum, Spathiphyllum cochlearispatum, Monstera pertusa, Anthurium magnificum, Anthurium hookeri, Anthurium elegans, Calathea zebrina, Yucca elephantipes, Bromelia balansae, Musa textilis, Myrthus communis, Olea oleaster, Olea europaea, Nerium oleander, Cocculus laurifolius, Microsorium punctatum, Sanseviera sp., Adansonia digitata, Boehmeria biloba, Piper nigrum, Phymatosorus scolopendria, Tumera ulmifolia, Nicodemia diversifolia, Tapeinochilos spectabilis, Rauwolfia tetraphylla, Ficus elastica, Cycas circinalis, Caryota urens, Cynnamomum zeylonicum, Aechmea luddemanniana, Phoenix zeylonica, Ficus benjamina, Ficus pumila, Murraya exotica, Trevesia sundaica, Clerodendrum speciosissimum, Actinidia kolomikta, Paeonia lactiflora, Paeonia suffruticosa, Quercus imbricaria, Iris pallida, Portulacca olleracea, Polygonum aviculare, Iris pseudocarpus, Allium nutans, Allium fistulosum, Anthericum ramosum, Veratrum nigrum, Polygonum lapathifolium, Hosta lancifolia, Hosta sieboldii, Echinops sphaerocephalus, Paeonia dahurica, Inula helenium, Crambe pontica, Digitalis lutea, Baptisia australis, Aristolochia australis, Hyssopus seravschanicus, Teucrium chamaedrys, Sedum album, Heracleum pubescens, Origanum vulgare, Cachrys alpina, Laser trilobum, Matteuccia struthiopteris, Sedum telephium, Bocconia cordata, Ajuga reptans, Thalictrum minus, Anemone japonica, Clematis rectae, Alchemilla officinalis, Potentilla alba, Poterium sangiusorba, Menispermum dauricum, Oxybaphus nyctagineus, Armoracia rusticana, Crambe cordifolia, Agrimonia eupatoria, Anchusa officinalis, Polemonium caeruleum, Valeriana officinalis, Pulmonaria molissima, Stachys lanata, Coronilla varia, Platycarya grandiflora, Lavandula officinalis, Vincetoxicum officinale, Acalypha hispida, Gnetum gnemon, Psychotria nigropunctata, Psychotria metbacteriodomasica, Codiaeum variegatum, Phyllanthus grandifolius, Pterigota alata, Pachyra affinis, Sterculia elata, Philodendron speciosum, Pithecellobium unguis-cati, Sanchezia nobilis, Oreopanax capitatus, Ficus triangularis, Kigelia pinnata, Piper cubeba, Laurus nobilis, Erythrina caffra, Metrosideros excelsa, Osmanthus fragrans, Cupressus sempervirens, Jacobinia sp., Senecio platyphylloides, Livistona chinensis, Tetraclinis articulata, Eucalyptus rudis, Podocarpus spinulosus, Eriobotrya japonica, Gingko biloba, Rhododendron sp., Thuja occidentalis, Fagopyrum suffruticosum, Geum macrophyllum, Magnolia kobus, Vinca minor, Convallaria majalis, Corylus avellana, Berberis sp., Rosa multiflora, Ostrya carpinifolia, Ostrya connogea, Quercus rubra, Liriodendron tulipifera, Sorbus aucuparia, Betula nigra, Castanea sativa, Bergenia crassifolia, Artemisia dracunculus, Ruta graveolens, Quercus nigra, Schisandra chinensis, Betula alba, Sambucus nigra, Gentiana cruciata, Encephalartos horridus, Phlebodium aureum, Microlepia platyphylla, Ceratozamia mexicana, Stenochlaena tenuifolia, Adiantum trapeziforme, Adiantum raddianum, Lygodium japonicum, Pessopteris crassifolia, Asplenium australasicum, Agathis robusta, Osmunda regalis, Osmundastrum claytonianum, Phyllitis scolopendrium, Polystichum braunii, Cyrtomium fortunei, Dryopteris filix-mas, Equisetum variegatum, Athyrium nipponicum, Athyrium filix-femina, Parthenocissus tricuspidata, Ligusticum vulgare, Chamaecyparis pisifera, Rosa canina, Cotinus coggygria, Celtis occidentalis, Picea schrenkiana, Cydonia oblonga, Ulmus pumila, Euonymus verrucosus, Deutzia scabra, Mespilus germanica, Quercus castaneifolia, Euonymus europea, Securinega suffruticosa, Koelreuteria paniculata, Syringa josikaea, Zelkova carpinifolia, Abies cephalonica, Taxus baccata, Taxus cuspidata, Salix babylonica, Thuja occidentalis, Actinidia colomicta, Mahonia aquifolium, Aralia mandschurica, Juglans nigra, Euonymus elata, Prinsepia sinensis, Forsythia europaea, Sorbocotoneaster pozdnjakovii, Morus alba, Crataegus macrophyllum, Eucommia ulmifolia, Sorbus commixta, Philodendron amurense, Cornus mas, Kerria japonica, Parrotia persica, Jasminum fruticans, Swida sanguinea, Pentaphylloides fruticosa, Sibiraea altaiensis, Cerasus japonica, Kolkwitzia amabilis, Amigdalus nana, Acer mandschurica, Salix tamarisifolia, Amelanchier spicata, Cerasus mahaleb, Prunus cerasifera, Corylus avellana, Acer tataricum, Viburnum opulus, Syringa vulgaris, Fraxinus exelsior, Quercus trojana, Chaenomeles superba, Pinus salinifolia, Berberis vulgaris, Cotoneaster horisontalis, Cotoneaster fangianus, Fagus sylvatica, Pinus pumila, Pinus sylvestris and Berberis thunbergii.

An aspect of the invention provides a method for eliciting a compound having therapeutic activity from a plant or plant part, comprising the steps of: contacting a living, intact plant or plant part with an effective amount of acetic acids; and allowing the acetic acid to induce or improve the production of a compound from the plant or plant part. A preferred acetic acid concentration is about 0.1% (v/v) and a preferred aqueous medium is water. A preferred plant portion is a plant root, although leaves and shoots are also contemplated for use in the methods according to the invention. A preferred therapeutic activity is an anti-microbial activity (such as an anti-bacterial activity or an anti-fungal activity) or an anti-cancer activity.

In a related aspect, the invention contemplates a method for eliciting a compound having therapeutic activity from a plant or plant part that further comprises recovering the compound from the plant or plant part. For example, the recovery step may comprise extracting or exuding the compound into an aqueous medium and collecting the compound from the aqueous medium. In addition, the recovery step may involve extracting that comprises macerating the plant or plant parts in an aqueous medium. Alternatively, the step of extracting the compounds may comprise removing cuticular material located on the surface of a leaf by contacting the leaf surface with a solvent, such as an organic solvent (e.g., methylene chloride or chloroform). Compounds recovered in this manner include, but are not limited to, lipid, wax, cutin, protein, a primary metabolite and/or a secondary metabolite. In some embodiments of the invention, the medium is a liquid medium; in other embodiments, the medium is an agar medium.

In a further related aspect, the above-described method for eliciting a compound having therapeutic activity from a plant or plant part further comprises providing a chemical library of compounds recovered from the aqueous medium in an amount sufficient to assay for biological activity. In some embodiments, the method further comprises assaying the solvent solution for therapeutic activity, such as anti-microbial activity (e.g., anti-bacterial or anti-fungal activity) or anti-cancer activity. In another embodiment, the step of assaying the solvent solution comprises contacting the solution with a medium containing a living microorganism and determining the rate of growth of the microorganism, whereby an inhibition of the growth of the microorganism is indicative of an agent in the solvent solution having therapeutic activity.

Another aspect of the invention provides a method of preparing a composition having therapeutic activity, comprising the steps of: contacting a living intact plant or plant part with an effective amount of acetic acid; allowing the acetic acid to induce a compound or component having therapeutic activity from the plant or plant part; and collecting a composition comprising the compound or component. A preferred concentration of acetic acid for use in the method is about 0.1% (v/v). In one embodiment, the composition is collected by contacting a surface of the plant or plant parts with a solvent suitable for removing cuticular or epicuticular material. In another embodiment, the composition is collected by macerating the plant or plant parts in an aqueous medium.

Numerous additional aspects and advantages of the invention will become apparent to those skilled in the art upon consideration of the following detailed description of the invention.

### BRIEF DESCRIPTION OF THE DRAWING

FIG. 1 is a graphical presentation of the amount of daidzein recovered from root exudates produced by soybean plants treated with different elicitors in accordance with an embodiment of the invention.

FIG. 2 is a graphical presentation of the amount of genistein recovered from root exudates produced by soybean plants treated with different elicitors in accordance with an embodiment of the invention.

FIG. 3 is an HPLC profile of the diversity of compounds recovered from root exudates of various plants in accordance with an embodiment of the invention.

FIG. 4 is an HPLC profile of the diversity of compounds recovered from Lupinus luteus in accordance with an embodiment of the invention.

FIG. 5 is an HPLC profile of the diversity of compounds recovered from root exudates of Brassica juncea in accordance with an embodiment of the invention.

FIG. 6 is an HPLC profile of the diversity of compounds recovered from root exudates of Datura metel in accordance with an embodiment of the invention.

FIG. 7 is an HPLC profile of the diversity of compounds recovered from root exudates of Lupinus polyphyllus in accordance with an embodiment of the invention.

FIG. 8 is an HPLC profile of the diversity of compounds recovered from root exudates of Melilotus medicaginoides in accordance with an embodiment of the invention.

FIG. 9 is an HPLC profile of the diversity of compounds recovered from Solanum melongena without treating with an elicitor.

FIG. 10 is an FPLC profile of the diversity of compounds recovered from root extracts from Solanum melongena which was treated with an elicitor.

FIG. 11 is an HPLC profile of the diversity of compounds recovered from Daucus carota which was treated with an elicitor.

FIG. 12 is an HPLC profile of the diversity of compounds recovered from Glycyne max which was treated with elicitors, as compared to a control.

FIG. 13 is an HPLC profile of the diversity of compounds recovered from Daucus carota which was treated with elicitors, as compared to a control.

FIG. 14 is an HPLC profile of the diversity of compounds recovered from Daucus carota which was treated with elicitors, as compared to a control.

FIG. 15 is an HPLC profile of the diversity of compounds recovered from Lycopersicon aesculentum which was treated with elicitors, as compared to a control.

FIG. 16 is an HPLC profile of the diversity of compounds recovered from Lupinus polyphyllus which was treated with elicitors, as compared to a control.

FIG. 17 is a representation of an agar plate showing antimicrobial activity of the root exudate of Laurus nobilis (1881) against Escherichia coli.

FIG. 18 is a representation of an agar plate showing antimicrobial activity of the root exudate of Gentiana tibetica (1881) against Escherichia coli.

FIG. 19 is a representation of an agar plate showing antimicrobial activity of the root exudate of Aconitum napellus (1881) against Escherichia coli.

FIG. 20 is a representation of an agar plate showing antimicrobial activity of the leaf surface compounds (identified on the Figure as samples) of Erythrina christa-galli (1363) against Staphylococcus aureus.

FIG. 21 is a representation of an agar plate showing antimicrobial activity of the leaf surface compounds (identified on the Figure as samples) of Laurus nobilis (1513) against Staphylococcus aureus.

FIG. 22 is a representation of an agar plate showing antimicrobial activity of the root exudate of Scutellaria altissima (1671) against Staphylococcus aureus.

FIG. 23 is a representation of an agar plate showing antimicrobial activity of the root exudate of Scuttellaria creticola (1691) against Staphylococcus aureus.

FIG. 24 is a representation of an agar plate showing antimicrobial activity of the root exudate of Hosta fortunei (1645) against Saccharomyces cerevisiae.

FIG. 25 is a representation of an agar plate showing antimicrobial activity of the root exudate of Cunninghamia lanceolata (2489) against Aspergillus flavus.

FIG. 26 is a representation of an agar plate showing antimicrobial activity of leaf surface compounds (identified on the Figure as samples) from leaves of Thymus citriodorus “aureus” (59) and Hydrocotyle asiatica (32a) against Staphylococcus aureus.

FIG. 27 is a representation of an agar plate showing antimicrobial activity of leaf surface compounds (identified on the Figure as samples) from leaves of Betula pendula (24) against Staphylococcus aureus.

FIG. 28 is a representation of an agar plate showing antimicrobial activity of leaf surface compounds of Eucalyptus rudis (229) against Staphylococcus aureus.

FIG. 29 is a representation of an agar plate showing antimicrobial activity of leaf surface compounds of Eucalyptus rudis (229) against Saccharomyces cerevisiae.

FIG. 30 is a representation of an agar plate showing antimicrobial activity of leaf surface compounds of Oreopanax capitatus (216) against Staphylococcus aureus.

FIG. 31 is a representation of an agar plate showing antimicrobial activity of leaf surface compounds of Oreopanax capitatus (216) against Escherichia coli.

### DETAILED DESCRIPTION OF THE INVENTION

The plant used in the present invention may be an entire grown plant or a plant seed or seedling or a plant shoot or root. The plant or plant portion is maintained intact and alive and is capable of being sustained without either an organic nutritional supplement or without maintaining sterile conditions for the plant. If desired or preferred, inorganic supplements may be employed in order to increase plant growth, but are not necessary to the method of the invention.

Typically, the plant or plant part is grown and maintained in a growth state similar to that in a natural surrounding. The plant functions as a natural bioreactor for producing valuable plant products, which may be recovered by contacting the plant or plant part with an aqueous medium, for example water. Moreover, the plants are capable of being grown and maintained in a manner of continuous growth, i.e., without destroying the plant. The plant or plant parts can be grown without any organic supplements although an organic supplement could be used if desired. As such the plant or plant parts are different than plants or plant parts that are cultured in a laboratory which generally require organic nutritional supplements and sterile conditions in order to maintain growth.

As herein above indicated, the plants or plant parts maybe subjected to physical or chemical treatment to elicit or induce an increased production of one or more compounds. The plants, and particularly in the roots of the plant may be contacted with any elicitor or inducer. Physical elicitors can include treatment such as ultra-violet radiation; low and high temperature stress; osmotic stress, for example as induced by salt or sugars; and nutritional stress, such as depriving the plant of essential nutrients (N, P, or K). More specifically, compounds that have demonstrated effectiveness in inducing the production of compounds with therapeutic activity in plants include, but are not limited to, salicylic acid, acetic acid, silver nitrate, chitosan, N-hexanoyl homericinelactone, methyl jasmonate, tetcyclases, pentafluorobenzoic acid, dihydroxybenzoic acid, cinnamic acid, 2-fluorobenzoic acid, sodium fluoride, yeast extract, laminarin, SDS, jasmonic acid, okadaic acid, polygalacturonic acid, 1-phosphatidic acid, polyethylene glycol. hydrogen peroxide, paraquate, calyculin A, 1-aminobutyrate, eicosapentanoic acid, arachidonic acid, glutathione, ascorbic acid, nickel, copper, and lead. Chemical compounds that can elicit or induce compounds with therapeutic activity includes, but are not limited to organic and inorganic acids, fatty acids, glycerides, phospholipids, glycolipids, organic solvents, amino acids peptides, monosaccharides, oligosaccharides, polysaccharides, lipopolysaccharides, phenolics, alkaloids, terpenes, terpenoids, antibiotics, detergents, polyamines, peroxides, ionophores, and the like.

For example, a glycopeptide elicitor may be prepared from germ tubes of the rust fungus Puccinia graminis Pers. F. sp. Tritici Erkss. & Henn (Pgt), as well as chitin oligosaccharides, chitosan, and methyl jasmonate (MG) stimulated lipoxygenase (LOX) activity (E.C. 1.13.11.12) in wheat (Triticum aestivum) leaves. The effects of elicitor concentration and exposure time on growth and levels of biologically active compounds vary. For example, transient studies at the same level demonstrated possible catabolism as serpentine, tabersonine, and lochnericine levels decreased immediately after elicitation. The levels of these compounds recovered back to control levels or were higher than control levels after some time. Jasmonic acid was found to be a unique elicitor leading to an enhancement in flux to several branches in the indole alkaloid pathway. Jasmonic acid addition caused an increase in the specific yields of ajmalicine (80%), serpentine (60%), lochnericine (150%), and horhammericine (500%) in dosage studies. Tabersonine, the likely precursor of lochnericine and horhammericine, decreased at lower levels of jasmonic acid and then increased with increasing jasmonic acid concentration. Transient studies showed that lochnericine and tabersonine levels go through a maxima, then decrease back to control levels and reduce below control levels, respectively. The yields of ajmalicine, serpentine, and horhammericine increased continuously after the addition of jasmonic acid. The methods described in the invention could generally be used in devising strategies for enhancement in productivity of secondary metabolites and for probing and studying the complex secondary metabolite pathways in plant tissue cultures.

The elicitor treatment may be applied continuously or intermittently to the plant or plant part. In one embodiment, such treatment may be accomplished by contacting the plant roots with a solution containing the elicitor or by irradiating the roots or exposing them to other environmental stresses, for example temperature stresses; however, the invention is not limited to such an embodiment in that other portions of a plant or seedlings may be contacted with an elicitor.

One elicitor, acetic acid, demonstrates particular effectiveness for inducing the production of compounds having therapeutic activity in plants. The exposure of plants or plant parts to acetic acid induced compounds demonstrating cytotoxicity against breast, central nervous system, and lung cancer cells in at least 40% of plant species tested. In addition, at least 30% of plant species exposed to an acetic acid elicitor demonstrated antimicrobial activity, for example activity against gram-positive bacteria, gram-negative bacteria, or inhibition of yeast or fungal growth. While not intended to be limited by any theory of the invention, it is particularly interesting to note that the effects of elicitation by acetic acid in plants or plant parts is not limited to, or necessarily, correlated with any change in the pH of the plant environment, as would be expected by one of skill in the art. Moreover, although acetic acid may be a very basic precursor of the indole-3-acetic acid pathway in plants, to the best knowledge of the applicants the literature has neither suggested nor reported any utility of acetic acid as a precursor.

The elicitor is applied in any amount suitable for inducing or improving production of a compound having therapeutic activity in the plant or plant parts. As used herein, the term “effective amount” refers to any amount capable of inducing or improving particularly antibacterial, anti-fungal, or anticancer activity in the subject plant or plant parts.

The present invention may be employed for screening for potentially valuable products. The plant or plant part subjected to the elicitor treatments can be extracted into water or recovered from harvested roots as herein above described.

Generally, the living plant or plant part is contacted with water or other aqueous extraction medium. The water or extraction medium containing the secreted phytochemicals is then analyzed to ascertain whether or not the medium contains a potentially valuable chemical compound, i.e. a compound demonstrating therapeutic activity. The contact with water may be effected by placing at least the plant roots in water or by “aeroponics,” which involves contacting the plant, in particular, the roots of the plant, with water droplets from which chemical compounds is recovered. The living plant or plant part subjected to the process of the invention is an intact plant or plant part in the form of an entire plant or plant seedlings including for example seeds, leaves, or plant roots. In each case, the plant or plant part is contacted with water, which may be pure water or water containing appropriate additives, such as desired chemical elicitors. In the case where a whole plant is used, it is preferred that the plant roots are contacted with water in order to recover chemical products.

In accordance with a preferred aspect, the chemical products are those which are secreted or leached from the plant, either the seedling or a shoot, and in particular, the plant root. In the case where seedlings, seeds, or shoots are used, and the plant, seed, shoot or root may be treated with an elicitor or inducer to increase production in the plant of one or more products. The inducer or elicitor may be present in the water that contacts the plant for chemical recovery or may be separately applied to the plant in any suitable manner.

In accordance with one preferred aspect of the invention, secreted chemical products are recovered from a plant (or plant portions) in a continuous process contacting the plant or plant parts with an aqueous or in a medium continuous or periodical manner to dissolve the chemicals secreted by the plant or plant parts into the aqueous medium. The aqueous medium containing exuded or secreted chemical products is then treated to recover the products from the water.

Alternatively, the roots are harvested and the chemicals are recovered from the harvested root by extracting and macerating the root tissue in an aqueous medium. In such embodiment, the plant or rooted shoots may be grown hydroponically and the plants are cultivated on top of a mesh with a small portion of their root system anchored in a layer of artificial soil above the mesh. The artificial soil layer is employed to supply all essential nutrients to the plants. In such an embodiment, a major portion of the root system grows through the mesh and soil layer into water, or other aqueous medium, which is below the mesh layer. The water flows over the roots in order to recover exuded chemicals. The water containing the exuded chemicals is then treated to recover and/or isolate the chemicals therefrom. In another embodiment, the root is harvested and chemicals are recovered from the harvested roots.

In a further embodiment, plants may be germinated and supported in rockwool cubes, as known in the art, with roots extending into a water solution which typically contains the elicitor and any inorganic nutrients.

In another embodiment, plant seedlings are employed as a source of the phytochemicals. In such an embodiment, seeds are germinated in aerated water, with the water being recovered on a continuous or semi-continuous basis in order to treat the water to obtain therefrom secreted phytochemicals. Although the preferred embodiment is a continuous process, batch processes may also be employed for accomplishing the method of the invention.

Thus, in accordance with the present invention, plants or plant parts may be grown on a large scale and used to effectively generate a diverse library of compounds for screening for various applications. Such diversity may be obtained by using a variety of plants and/or a variety of elicitors. Such library may be effectively generated and screened in that such variety of compounds may be recovered as root exudates by a simple water extraction. Moreover, the extraction medium may also contain the elicitors which function to elicit various compounds in the root exudates.

Furthermore, the present invention provides a “factory” for large scale production of compounds in that desired compounds can be simply recovered in root exudates from plants or plant parts which are hydroponically grown. Moreover, by use of a selected elicitor, desired compounds which may not be normally present in root exudates and/or which may not be present in sufficient quantities in root exudates can be recovered on a large scale from root exudates produced by hydroponically grown plants or plant parts by a simple water extraction or by harvesting roots without destroying the plant.

The water or aqueous medium which contains the secreted chemicals also can be treated to recover the chemicals therefrom. The chemical compounds are recovered from the water or extraction medium used on harvested roots. For example, in one aspect, the invention provides a method of identifying an agent which is exuded onto the surface of a leaf of a plant having therapeutic activity. The method comprises (a) removing cuticular material located on the surface of the leaf, comprising contacting the leaf surface with a solvent, thereby resulting in a solvent solution; (b) assaying the solvent solution for the identification of an agent which has therapeutic activity; and (c) analyzing the solvent solution so as to identify the agent which has the therapeutic activity. The extraction medium is analyzed by various techniques in order to assist in identifying the recovered compounds. For example, the chemicals may be recovered by column chromatography, crystallization, distillation, liquid or solid phase extraction and the like. Such procedures are known in the art and should be apparent to those skilled in the art from the teachings herein.

The cuticular material can be a lipid, wax, cutin, protein, primary or secondary metabolite. Typically, the solvent is an organic solvent. Examples of solvents include, but are not limited to, methylene chloride and chloroform.

The step of analyzing the solution further can include fractioning the solvent solution directly or following any drying or resuspension step. In one embodiment, the resulting agent is identified subsequent to fractioning the solution. Fractionation methods are known to those skilled in the art. For example, chromatographic methods, for example HPLC, may be employed to identify the compound. In particular, the chromatography separation of extracted products may be employed with an HPLC-system consisting of Waters 996 Photodiode Array Detector (PDA) with usable UV range from 190 to 800 nm; a Waters 717 plus autosampler; two Beckman and a Beckman System Gold Analog Interface 406. The Beckman solvent delivery system is controlled by a NEC PC-8300 computer. Chromatography and spectral data is managed by Waters Millennium chromatography software, version 2.10, using a NEC Image 466es computer. All hardware components, except the solvent delivery system, are connected through a standard IEEE 488 communication system. Compounds are separated on a Waters Nova Pak® C-18 reverse phase column, 3.9×150 mm, 60 Å pore size, and 4 μm particle size. Prior to use, each batch of solvent A is digested under vacuum and ultrasonications for 5 minutes. The mobile phase flow is adjusted to 1 mL/min, and a gradient mode of separation is used for all separations. Compounds are detected with PDA detector or with Waters™ Thermabeam Mass Detector.

Further, one may fractionate the sample by chromatography techniques, followed by the chemical structure analysis using mass spectroscopy; infrared spectroscopy; or 1D or 2D nuclear magnetic resonance spectroscopy (proton or ¹³C). Fractionation and analysis methods also are known to those skilled in the art.

As used herein, the term “therapeutic activity” refers to a biological activity, typically selected from antibacterial activity, antifungal activity, or anticancer activity. Particularly, “anti-microbial activity” as used herein can identify anti-bacterial and anti-fungal activity. Therapeutic activity can be identified by contacting the solvent solution, aqueous medium, or its components with a media containing a suspension of a microorganism, wherein the inhibition of the growth of the suspension of the microorganism is indicative of an agent in the solvent solution having therapeutic activity. The medium may be a liquid media or an agar media. Microorganisms can include, but not limited to any bacteria or fungi that grow within or develop in or on the media. Inhibition of the growth is detected by standard means known to those skilled in the art. For example, the growth inhibition on agar may be measured in terms of zone of inhibition, which is known to those skilled in the art.

The microorganism can be selected from a gram positive or a gram negative bacteria, protozoan, fungus, or virus. As defined herein, “antimicrobials” means the spectrum of organisms against which they are active, whether they kill the organism or merely slow its growth and reproduction (i.e., cidal or static), and the biochemical system on which they exert their major biochemical action (e.g., inhibit protein synthesis or cell wall synthesis). Antimicrobials can include gram positive or gram negative bacteria and may even act against such organisms as Rickettsia, fungi, or protozoans. Examples of gram positive and gram negative bacteria are known to those skilled in the art. Microorganisms include, but are not limited to, Escherichia coli K-12 F, prototropic Str, Staphylococcus aureus subsp. Aureus, Pseudomonas aeruginosa, Saccharomyces cerevisiae, Aspergillus flavus, and Penicillium Nigra.

Two common applications of agar diffusion assays are potency testing of new production lots in the pharmaceutical industry and bacterial susceptibility testing. Such applications are based on the same principles, but susceptibility assays are unknown bacterial strains, and potency assays use bacterial strains with well-characterized performance against the test drug. Agar diffusion potency assays are relatively comparable in sensitivity and accuracy to radiometric (RIA) enzyme, fluorescent (FIA) enzyme, & ELISA. For example, single-plate assay is based on having all standard and unknown concentrations on one single plate. This eliminates plate to plate variation, and facilitates layout and reading. Alternatively, 2 or 3 identical plates may be laid out, read and averaged. Usually NUNC (Denmark) large 24.3 mm square plates are used, that allow up to 64 samples that easily fit on one plate. As such, the assay provides for up to 6 standards, and up to 10 samples, with 4 replicates of each standard and unknown sample concentrations, on one plate. Multiple-Plate format uses many 90-100 mm petri dishes, and conforms strictly to US-FDA, US-CFR, and US-USP published methodology. Zone diameters are measured by Video or Caliper directly into the software.

In one embodiment the plant is a higher plant. The use of any plant is contemplated and may be employed in the method of the invention. For example, the following plants may be employed in order to identify agents which are produced by elicitation: Atropa belladonna, Erythrina flabelliformis, Ipomoea tricolor, Erythrina crista, Celosia cristata, Gallium spurium, Laurus nobilis, Vitis labrusca, Gratiola officinalis, Symphitum officinalis, Hosta fortunei, Cassia hebecarpa, Thalictrum flavum, Scutellaria altissima, Portulacca oleracea, Scutellaria certicola, Physalis creticola, Geum fauriei, Gentiana tibetica, Linum hirsutum, Aconitum napellus, Podophyllum emodii, Thymus cretaceus, Hosta fortunei, Carlina acaulis, Chamaecrista fasciculata, Pinus pinea, Peganum harmala, Tamarindus indica, Carica papaya, Cistus incanus, Capparis inermis, Cupressus lusitanica, Diospyros kaki, Eryngium campestre, Aesculus woerlitzensis, Aesculus hippocastanum, Cupressus sempervirens, Celtis occidentalis.

Further plants species used for screening are as follows: Polygonum cuspidatum, Elaeagnus angustifolia, Elaeagnus commutata, Gentiana macrophylla, Brassica rapa, Sesbania exaltata, Sesbania speciosa, Spartina potentiflora, Brassica juncea, Helianthus annus, Poinsettia sp., Pelargonium zonale, Leontopodium alpinum, Lupinus luteus, Buxus microphylla, Liatris spicata, Primula japonica, Betula nigra, Filipendula vulgrais, Lobelia siphilitica, Grevillea robusta, Reseda luteola, Gentiana littoralis, Campanula carpatica, Aesculus hippocastanum, Aesculus woerlitzensis, Ageratum conizoides, Psidium guajava, Ailanthus altissima, Buxus microphylla, Hydrocotyle asiatica, Grevillea robusta, Brugmansia suaveolens, Thymus pulegioides, Thymus lema-barona, Thymus serphyllum, Gaultheria procumbens, Thymus camosus, Thymus thracicus, Calycanthus floridus, Zingiber officinalis, Lamium dulcis, Thymus praecox, Thymus pulegioides, Thymus speciosa, Thymus carnosus, Thymus pseudolamginosus, Thymus vulgraris, Ficus religiosa, Forsythia suspensa, Chelidonium majus, Thymus wooly, Thymus portugalense, Nicotiana tabacum, Thymus cytridorus, Cactus officinailis, Lablab purpurea, Juglans regia, Actinidia chinensis, Hemerocallis, Betula pendula, Gardenia jasminoides, Taxodium distichum, Magnolia loebherii, Crataegus praegophyrum, Larix decidua, Thuja orientalis, Thuja occidentalis, Thuja orientalis, Cupressocyparis leylandii, Pseudotsuga menziesii, Abies firma, Parthenocissus quinquefolia, Allium cemnuum, Juniperus “blue pacific”, Taraxacum officinalis, Yucca sp., Ilex aquifolium, Tsuga canadensis, Ilex comuta, Taxus hiksii, Taxus media, Metasequoia glyptostroboides, Pinus bungiana, Buxus sempervirens, Stewartia koreana, Prunus sp., Betula dahurica, Plantago minor, Acer palmatum, Acer campestre, Cotynus coggygria, Quercus robur, Acer truncatum, Achyranthes bidentata, Allium japonicum, Carum capsicum, Agastache mexicana, Prunella vulgaris, Tagetes minuta, Nepeta cataria, Ratibida columnifera, Aster novae angliae, Myrica cerifera, Pittosporum tobira, Taxodium distichum, Plantago major, Pinus sylvestris, Acorus canadensis, Pieris japonica, Pinus strobus, Trifolium pratense, Prunus serotica, Datura stramonium, Geranium maculata, Hydrocotyle asiatica, Taxodium distichum, Astragalus sinicus, Centauria maculata, Ruschia indurata, Myrthus communis, Platanus occidentalis, Licium barbatum, Lavandula officinalis, Grevillea robusta, Hippophaee rhamnoides, Filipendula ulmaria, Betula pendula, Polygonum odoratum, Brugmansia graveolens, Rhus toxicodenta, Armoracia rusticana, Ficus benjaminii, Sluffera sp, Pelagonium zonale, Allium sp, Asimina triloba, Lippa dulcis, Epilobium augustifolium, Brugmansia suavecolens (old), Brugmansia suaveolens (young), Xanthosoma sagittifolium. (leaf), Xanthosoma sagittifolium (stem), Monstera deliciosa., Aglaonema commutatus, Dieffenbachia leopoldii, Anthurium andreanum, Syngonium podophyllum, Dracaena fragrans, Ananas comosus, Strelitzia reglinae, Diffenbachia segiunae, Syngonium aurutum, Dracaena sq, haemanthus katharina, Anthurium altersianum, Spathiphyllum grandiflorum, Spathiphyllum cochlearispatum, Monstera, pertusa, Anthurium magnificum, Anthurium hookeri, Anthurium elegans, Calathea zebrina, Yucca elephantipes, Bromelia balansae, Musa textilis (Leaf), MUSA textilis (Stem), Myrthus communis, Olea olcaster, Olea europaea, Verium oleander, Cocculus laurifolius, Microsorium punctatum, Ficus sp., Senseviera sp., Adansonia digitata, Boechimeria boloba, Piper nigrum, Phymatosorus scolopendria, Turnera ulmifolia, Nicodemia diversifolia, Tapeinochilos spectabilis, Rauwolfia tetraphylla, Ficus elastica, Cycas cirinalis, Caryota ureus, Cynnamonum zeylonicum, Aechmea luddemoniana, Foenix zeulonica, Ficus benjamina, Ficus pumila, Murraya exotica, Trevesia sungaica, Clerodendrum speciossicum, Actinidi colonicta, Paeonia lactiflora, Paeonia suffructicisa, Quercus imbricaria, Iris alida, Portulacca olleracea, Poligonum aviculare, Iris pseudocarpus, Allium nutans, Allium fistulosum, Antericum ramosum, Veratrum nigrum, Polygonum latifolia, Hosta lancefolia, Hosta zibalda, Echinops sphae, Paeonia dahurica, Inula hilenium, Trambe pontica, Digitalis lutea, Bactisia australis, Austolachia australis, Hissopus zeraucharicus, Feucrium ham. edris., Sedum album, Heraclelum pubescens, Origanum vulgare, Cachris alpina, Haser trilobum, Matteuccia struthiopteris, Sedum telchium, Bocconia cordata, Ajuga reptans, Thalictrum minus, Anemona japonica, Clematis rectae, Thalictrum sp., Alchemilla sp., Potentilla alba, Poterium sangiusorba, Menisperrnum dauricum, Oxybaphus nyctagineus. Armoracea rusticana, Crambe cordifolia. A rimonia eupatora, Anchusa officinalis, Poly monium ceruleum, Valeriana officinalis, Pulmonaria molissima, Stachys lanata, Coronilla varia, Platycarya grandiflora, Lavandula officinalis, Vincetoxicum officinale, Acalypha hispida, Gnetum gnemon, Psychotria nigropunctata, Psychotria metbacteriodomasica, Codiaeum variegatum, Phyllanthus grandifolius, Pterigota alata, Pachyra affinis, Sterculia elata, Philodendron speciosum, Pithecellobium unguis-cati, Sanchezia nobilis, Oreopanax capitatus, Ficus triangularis, Kigelia pinnata, Piper cubeba, Laurus nobilis, Erythrina caffra, Metrosideros excelsa, Osmanthus fragrans, Cupressus sempervirens, Jacobinia sp., Senecio platyphylloides, Livistona chinensis, Tetraclinis articulata, Eucalyptus rudis, Podocarpus spinulosus, Eriobotrya japonica, Gingko biloba, Rhododendron, Thuja occidentalis, Fagopyrum suffruticosum, Geum macrophyllum, Magnolia kobus, Vinca minor, Convallaria majalis, Corylus avellana, Berberis sp., Rosa multiflora, Ostrya carpinifolia, Ostrya connogea, Quercus rubra, Liriodendron tulipifera, Sorbus aucuparia, Betula nigra (leaf), Betula nigra (flower), Castanea sativa, Bergenia crassifolia, Artemisia dracunculus, Ruta graveolens, Quercus nigra, Schisandra chinensis, Betula alba, Sambucus nigra, Gentiana cruciata, Encephalartos horridus, Phlebodium aureum, Microlepia platyphylla, Ceratozamia mexicana, Stenochlaena tenuifolia, Adiantum trapeziforme, Adiantum raddianum, Lygodium japonicum, Pessopteris crassifolia, Asplenium australasicum, Agathis robusta, Osmunda regalis, Osmundastrum claytonianum, Phyllitis scolopendrium, Polystichum braunii, Cyrtomium fortunei, Dryopteris filix-mas, Equisetum variegatum, Athyrium nipponicum, Athyrium filix-femina, Parthenocissus tricuspidata, Ligusticum vulgare, Chamaecyparis pisifera, Rosa canina, Cotinus coggygria, Pinus strobus, Celtis occidentalis, Picea schrenkiana, Cydonia oblonga, Ulmus pumila, Euonymus verrucosus, Deutzia scabra, Mespilus germanica, Quercus castaneifolia, Euonymus europea, Securinega suffruticosa, Koelreuteria paniculata, Syringa josikaea, Zelkova carpinifolia, Abies cephalonica, Taxus baccata, Taxus cuspidata, Salix babylonica, Thuja occidentalis, Actinidia colomicta, Mahonia aquifolium, Aralia mandschurica, Juglans nigra, Euonymus elata, Prinsepia sinensis, Forsythia europaea, Sorbocotoneaster pozdnjakovii, Morus alba, Crataegus macrophyllum, Eucommia ulmifolia, Sorbus commixta, Philodendron amurense, Cornus mas, Kerria japonica, Parrotia persica, Jasminum fruticans, Swida sanguinea, Pentaphylloides fruticosa, Sibiraea altaiensis, Cerasus japonica, Kolkwitzia amabilis, Amigdalus nana, Acer mandschurica, Salix tamarisifolia, Amelanchier spicata, Cerasus mahaleb, Prunus cerasifera, Corylus avellana, Acer tataricum, Viburnum opulus, Syringa vulgaris, Fraxinus exelsior, Quercus trojana, Chaenomeles superba, Pinus salinifolia, Berberis vulgaris, Cotoneaster horisontalis, Cotoneaster fangianus, Fagus sylvatica, Pinus pumila, Pinus sylvestris, and Berberis thunbergii.

The invention will be further described with respect to the following examples. The examples are intended to provide an illustration of the invention and should not be construed as a limitation of the invention in any way.

### EXAMPLES

### Example 1

### Plant Production

Seeds were germinated in a greenhouse equipped with supplementary lighting (16-h photoperiod 24-28° C.). Seeds were placed inside 0.9 cm diameter, 0.9 cm deep well drilled in Grodan rockwool cubes (3.4 cm width×3.4 cm depth×3.7 cm height) purchased from Grodania A/S, Hedehousene, Denmark.

Depending on the speed of germination, the seeds were either placed directly into the rockwool cubes or sterilized to prevent rotting during the germination process. For sterilization, seeds were immersed first in 70% ethyl-Alcohol for 10-15 seconds, then in 2.5% Sodium Hypochlorite for 10-15 min., and finally rinsed thoroughly with distilled water. The sterilized seeds were placed in a Petri dish lined with no. 1 Wattinan paper (Wattman International Ltd., Maidstone, England), soaked in either a sterile water for seeds larger than 1 mm in diameter, or for smaller seeds with mineral salts nutrient solution. The Petri dishes were sealed with parafilm before being placed in a growth chamber (12-h photoperiod 22-24° C.) until the seeds germinated.

Rockwool cubes were placed inside standard greenhouse plastic trays (dimensions 52 cm width×25 cm depth×7 cm height) and watered with an intermittently operating overhead misting system triggered by a moisture sensor (Mist-A-Matic, E.C. Geiger Inc., Larleysville, Pa.). Seeds were allowed to germinate for 3-6 days till the roots started to emerge from the bottom of the rockwool cube.

After germination, the cubes with the seedlings were inserted into a 3.2 cm diameter round opening cut in the center of Styrofoam ring (8.2 cm diameter, 2.5 cm thickness). The ring was floated on the surface of 400-800 mL of hydroponic nutrient solution (2 g/L Hydro-Sol [Scotts-Sierra Horticultural Products Comp., Marysville, Ohio] supplemented with 1.2 g/L Ca[NO₃]₂) containing inside light impermeable, high-density polyethylene cylinder (9.0 cm in diameter, 16 cm in height).

Aeration was provided either by shaking the cylinders at 50 rpm on the platform shaker (Model Orbit, Lab-Line Instruments, Inc., Melrose Park, Ill.) or by bubbling compressed air through the solution. Seedlings were cultivated hydroponically in this system for 3 to 5 weeks with roots growing in a nutrient solution. Thereafter, the root system (average root dry weight 0.1±0.05 g) was removed from the nutrient solution and placed inside a 30 mL glass beaker, containing 10-20 mL of distilled water or distilled water supplemented with the elicitor. To prevent the water loss from the plant canopy and the drying of the collecting solution, shoots of the plants were covered with a plastic bag. After 24 h, unless noted otherwise, a small sample from the root solution was removed and analyzed for the phytosecreted products.

### Example 2

### Flow-through Phytosecretion System

The flow-through phytosecretion system consisted of a stainless steel container (53 cm width×34 cm depth×20 cm height) with 15-24 soybean plants (glycine max) supported by the rockwool cubes inserted in the openings in the Styrofoam raft (5.0 cm thickness) which had dimensions slightly smaller than the internal dimensions of the container. This Styrofoam raft was floating on top of approximately 10 L of nutrient solution (2 g/L Hydro-Sol supplemented with 1.2 g/L Ca(NO₃)₂, aerated with compressed air supplied through an air hose placed on the bottom of the container. After 4-5 weeks, or when the roots reached the appropriate size [PLEASE DEFINE—SIZE EQUIVALENT TO_ROOTS GROWN AS DESCRIBED FOR 4-5 WEEKS?], the volume of nutrient solution was reduced to 2 L. The flow of the nutrient solution, with or without an elicitor, through the flow through system was maintained with a peristaltic pump (Variable Flow Mini-Pump, Fisher Scientific, Pittsburgh, Pa.), which allowed easy adjustments in the volume of the solution entering the system. Typically, flow rates used in the experiments ranged from 1.5 to 4.5 L/day. The intake tube of the peristaltic pump was immersed in a 60 L plastic storage container containing nutrient solution. Solution from the storage container dripped into the pbytosecretion system through the tube attached to its wall. When necessary, elicitors were added to the storage container at the desired concentration. The solution was discharged from the phytosecretion container in the side opposite to the inlet through the opening cut in the bottom of the container. Solution level in the phytosecretion container was adjusted by changing the height of the opening of the outlet tube. Solution samples were taken from the end of the outlet tube at the specific intervals and analyzed for the presence of the phytosecreted compounds.

### Example 3

### High-pressure Liquid Chromatography (HPLC) Analysis of Phytosecreted Natural Products (isoflavonoids)

Soybean (glycine Max) seeds were germinated and a root solution was removed as described in Example 1.

An HPLC method for separation and identification of phytosecreted compounds, using isoflavonoids daidzein and genistein is used as an example. The chromatography separation was performed with an HPLC-system consisting of Waters 996 Photodiode Array Detector (PDA) with usable UV range from 190 to 800 nm; a Waters 717 plus autosampler; two Beckman 110B solvent Delivery Modules, connected with a Beckman System Organizer (mixer) and a Beckman System Gold Analog Interface Module 406. The Beckman solvent delivery system was controlled by a NEC PC-8300 computer. Chromatography and spectral data was managed by Waters Millennium chromatography software, version 2.10, using a NEC image 466es computer. All hardware components, except the solvent delivery system, were connected through a standard IEEE communication system. Isoflavonoid compounds were separated on a Waters Nova Pak® C-18 reverse phase column, 3.9×150 mm, 60 Å pore size, and 4 μm particle size.

The mobile phase consisted of two components: Solvent A-0.5% ACS grade acetic acid in double distilled water, pH 3-3.5; and Solvent B-acetonitrile. Prior to use, each batch of solvent A was degassed under vacuum and ultrasonication for 5 minutes.

The mobile phase flow was adjusted to 1 mL/min, and a gradient mode of separation was used for all separations. The gradient profile was as follows:

0-20min 0% B-100% B;

20-22 min 100% B;

22-25 min 100% B-0% B;

25-33 min 0% B (column equilibration for next injection).

Compounds were detected with PDA detector within the wavelength range of 200 to 400 mu. The column temperature was ambient.

Under the above conditions, daidzein had retention time 11.725 min and UV maxima at 250.9 nm and 302.9 nm and genistein had a RT of 12.94 min and UV maximum at 260.3 nm. Depending on the resolution setting of the PDA detector, a negligible shift of ±3 nm in the absorbance maxima was observed. A ±0.5 min of tolerance in the retention times with the different batches of solvents was detected.

All plants were grown hydroponically, as previously described, and phytosecreted compounds collected for 24 hours in distilled water containing an elicitor or mixtures of different elicitors, except for treatment no. 47, where no elicitors were present in the collecting water. Daidzein and genistein content in root exudates from un-elicited plants grown under the same conditions was below the detection limits −400 pg, or 4 μg/L for daidzein, and 25 pg, or 250 ng/L for genistein.

Elicitor Treatments

3-Salicylic acid (5 mM), Tetcyclases (0.2 mM) and 7.5% ethanol (EtOH)

5-Salicylic acid (5 mM) and 5% EtOH

6-Salicylic acid (2.5 mM) and 2.5% EtOH

7-Salicylic acid (5 mM) and 0.5 g/L SDS)

10-Salicylic acid (1 mM) and Pentafluorobenzoic acid (2 mM)

21-Silver nitrate (1 mM) in acidic pH (citric acid, pH 2.7)

24-Silver nitrate (2 mM) in acidic pH (acetic acid, pH 2.7)

25-Acetic acid (pH 2.7)

37-Pentafluorobenzoic acid (5 mM)

38-2.6-Dihydroxybenzoic acid (5 mM)

40-Cinnamic acid (16.5 mM) and 35.5% EtOH

42-Cinnamic acid (3.3 mM) and 7.1% EtOH

45-2-Fluorobenzoic acid (10 mM) and 2% EtOH

47-UV-light irradiation of the whole plant for 3 hours

55-Sodium fluoride (250 mM) and 10% EtOH

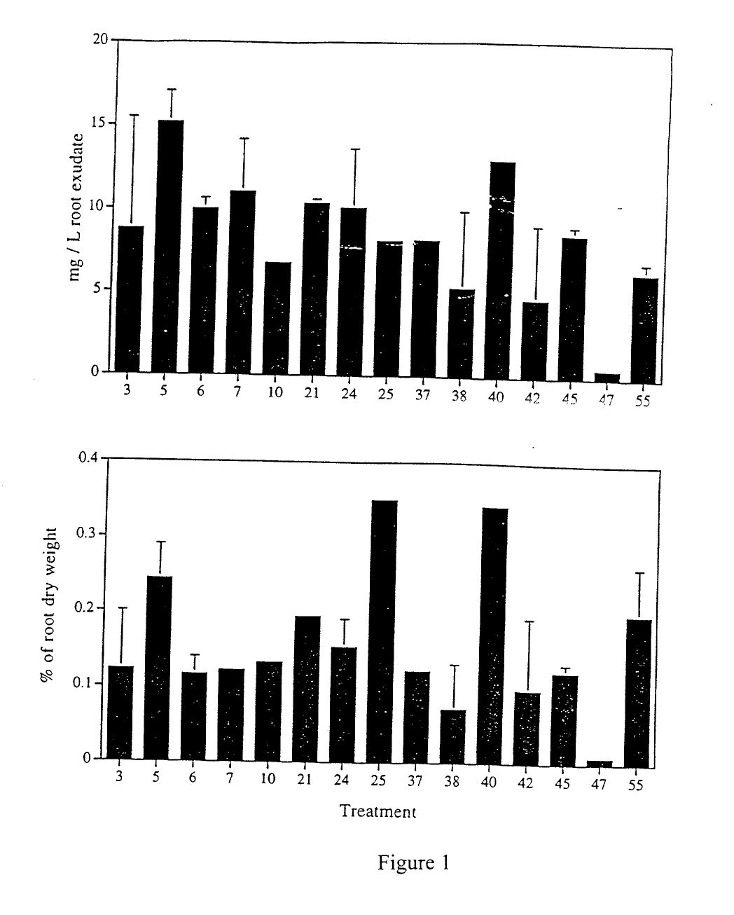

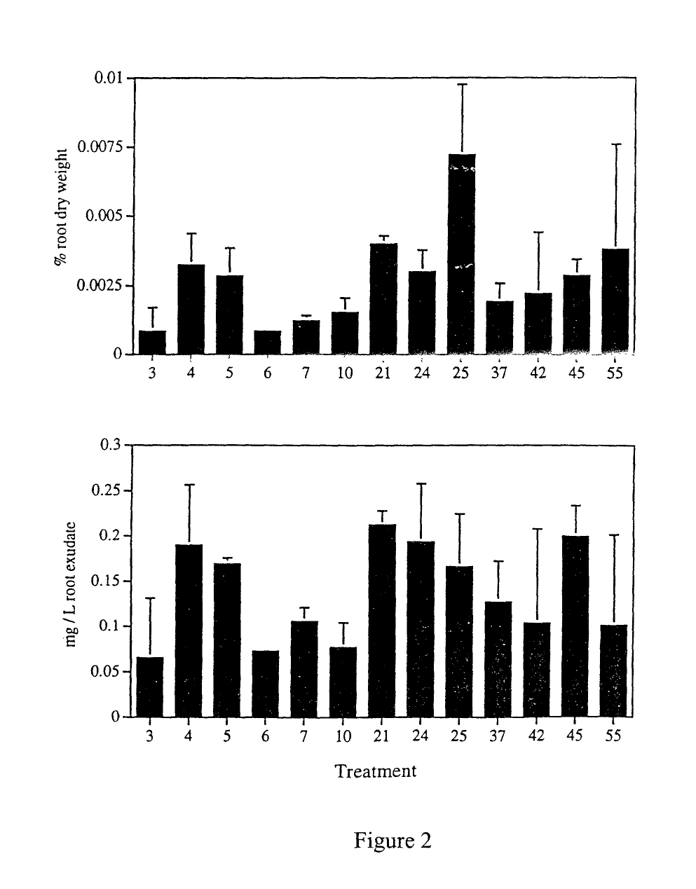

Fifteen treatments which elicited some of the highest levels of daidzein and genistein are shown in FIGS. 1 and 2 for simplicity. Some of the above-elicitors induced mild to moderate phytotoxicity in the treated plants. All compounds used in the mixtures produced significant levels of daidzein and genistein, when applied alone. However, combinations of various elicitors shown in FIGS. 1 and 2 usually produced higher levels of target compounds. Other compounds used as elicitors, such as yeast extract, laminarin, SDS, jasmonic acid, methyl jasmonate, okadaic acid, polygalacturonic acid, 1-phosphatidic acid, polyethylene glycol, hydrogen peroxide, paraquate, calyculin A, 1-amino butyrate, eicosapentanoic acid, arachidonic acid, glutathione, ascorbic acid, and some heavy metals (nickel, copper, lead) showed lower degree of elicitation of the target compounds.

Various plants were grown hydroponically as previous described and secreted compounds (root exudates) were collected in distilled water with and without an elicitor(s).

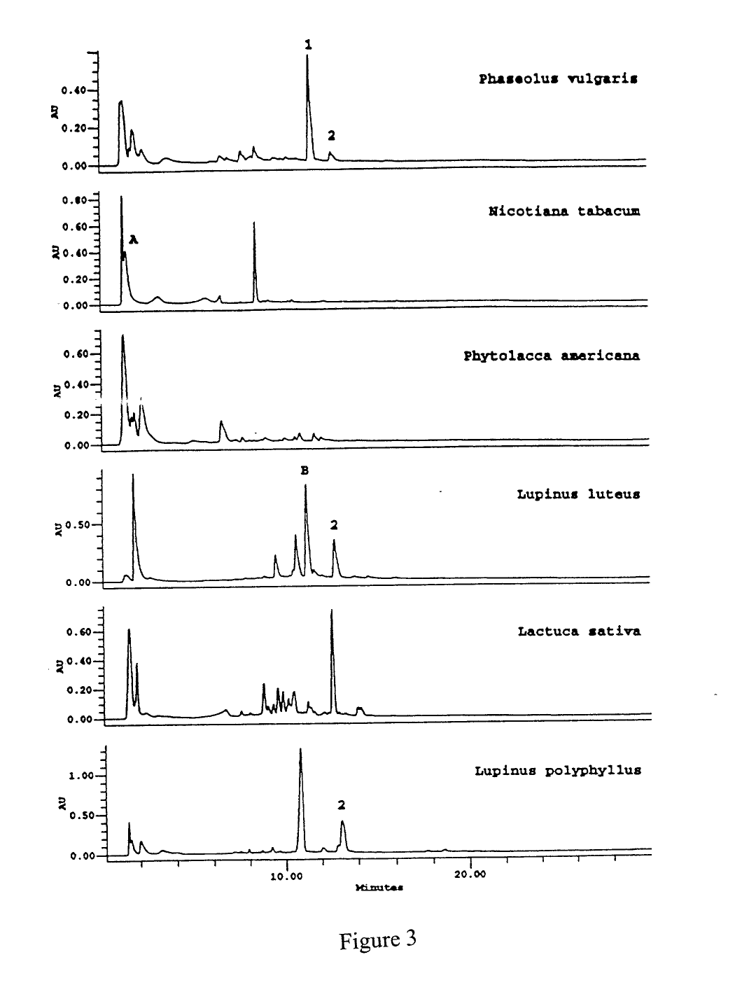

FIG. 3 is an HPLC profile of compounds recovered from the root exudates with UV detection at 251.8 nm. Most compounds were not identified, however, as shown in FIG. 3 the following designations in FIG. 3 were positively or putatively identified.

1. Positively identified Daidzein

2. Positively identified Genistein

A. Putatively identified Nicotine

B. Putatively identified at (5-0-methyl-genistein) Lupin luterus roots were not elicited; Lupinus polyphyllus roots were elicited with 2 mM Salicylic acid in 2% Ethanol; all other species were elicited with 0.3 M Acetic acid, pH 2.7.

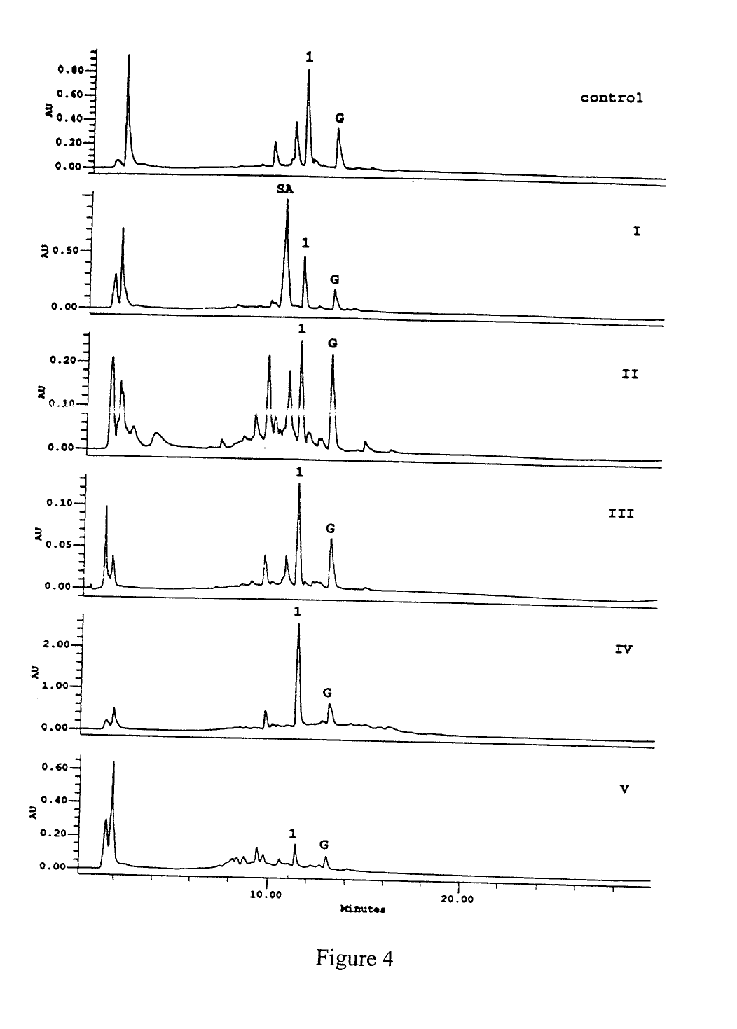

FIG. 4 demonstrates the diversity of compounds excreted from the roots of one plant species (Lupinus luterus) treated with different elicitors. (Note the large differences in the HPLC profiles of root exudates produced by different elicitors) UV detection at 251.8 nm.

Elicitor Treatments

control—No treatment

I—Treatment with 2 mM Salicylic acid in 2% Ethanol

II—Treatment with 0.3 M Acetic acid, pH-2.7

III—Treatment with 2 mM AgNO₃

IV—Treatment with 7.5 mM Arachidonic acid

V—Treatment with 5 mM Jasmonic acid

Most compounds were not identified. Those positively or putatively identified are:

1—Putatively identified as 5-0-Methyl-genistein

G—Positively identified as Genistein p0 SA—Salicylic acid

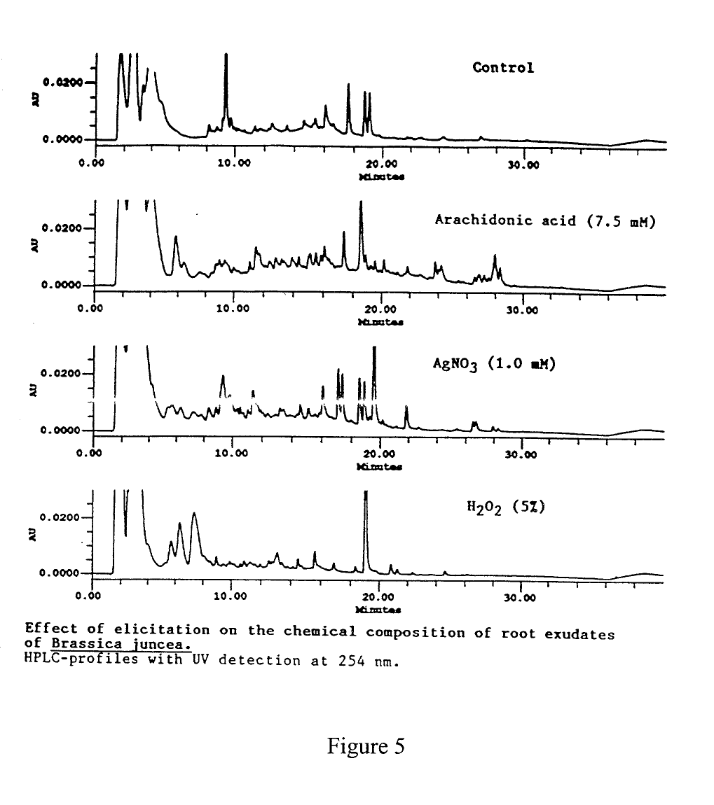

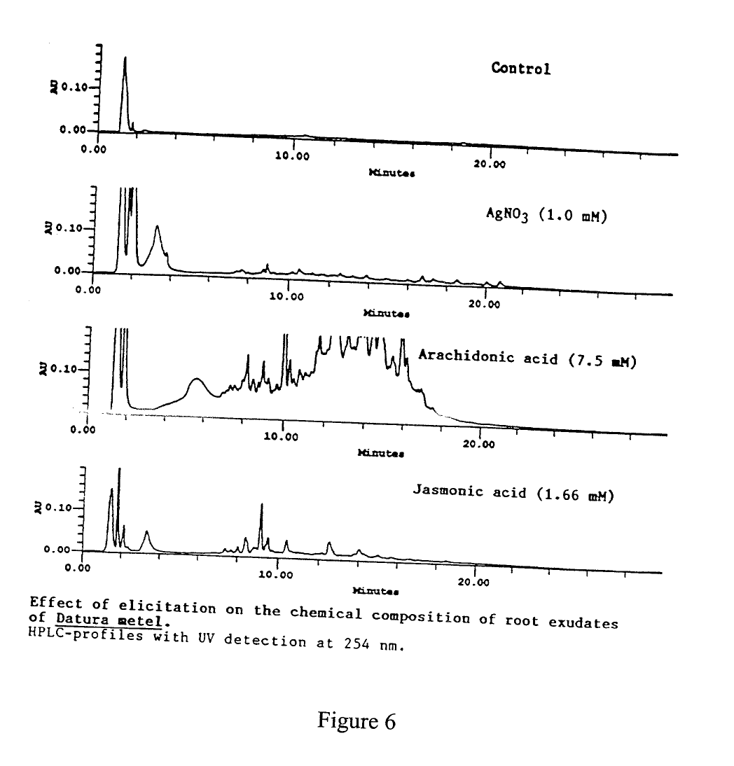

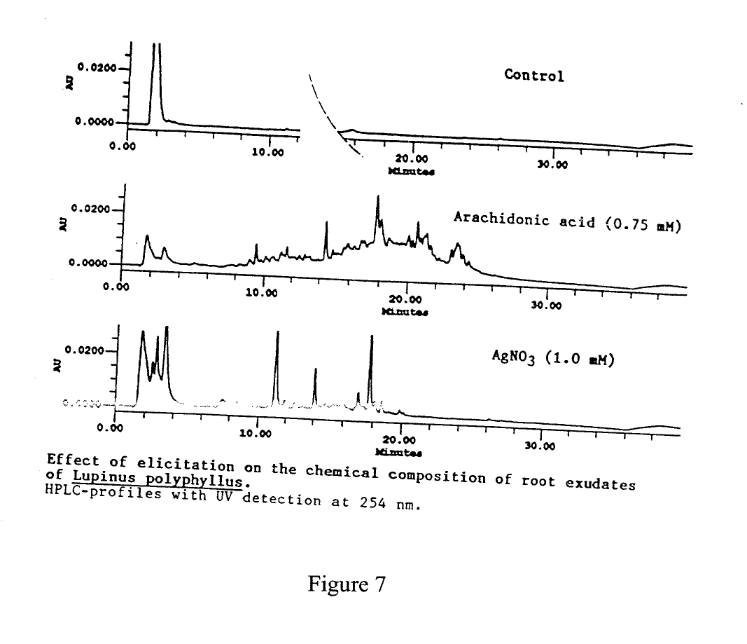

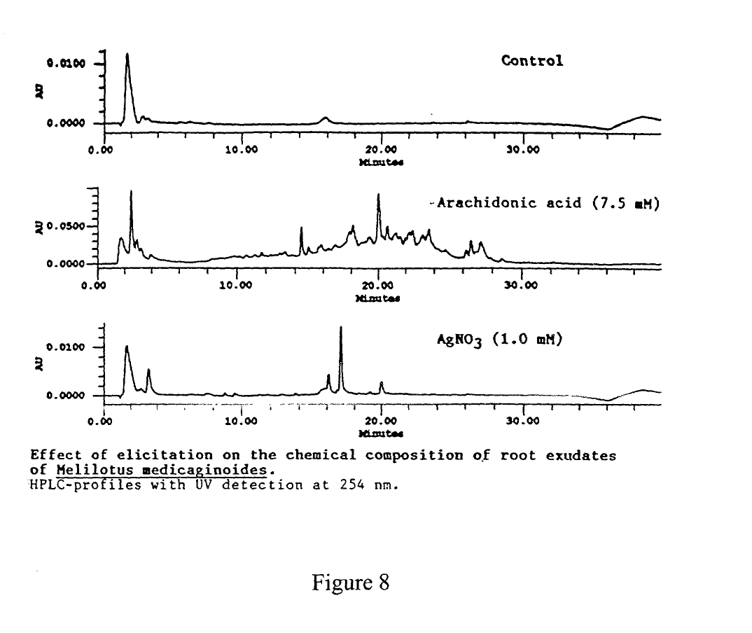

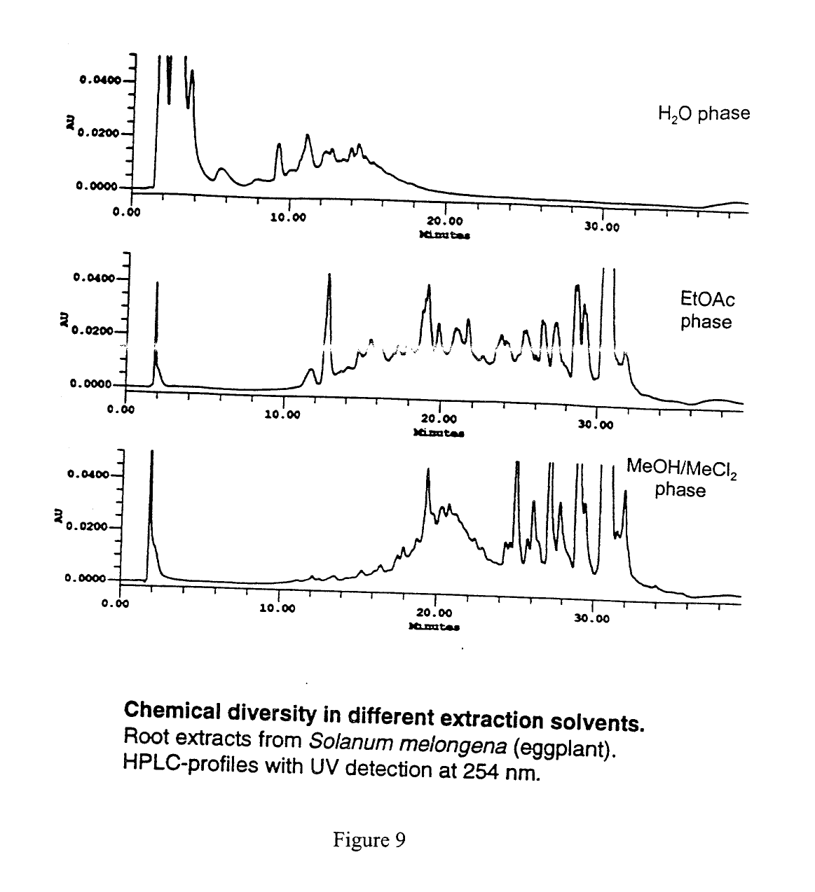

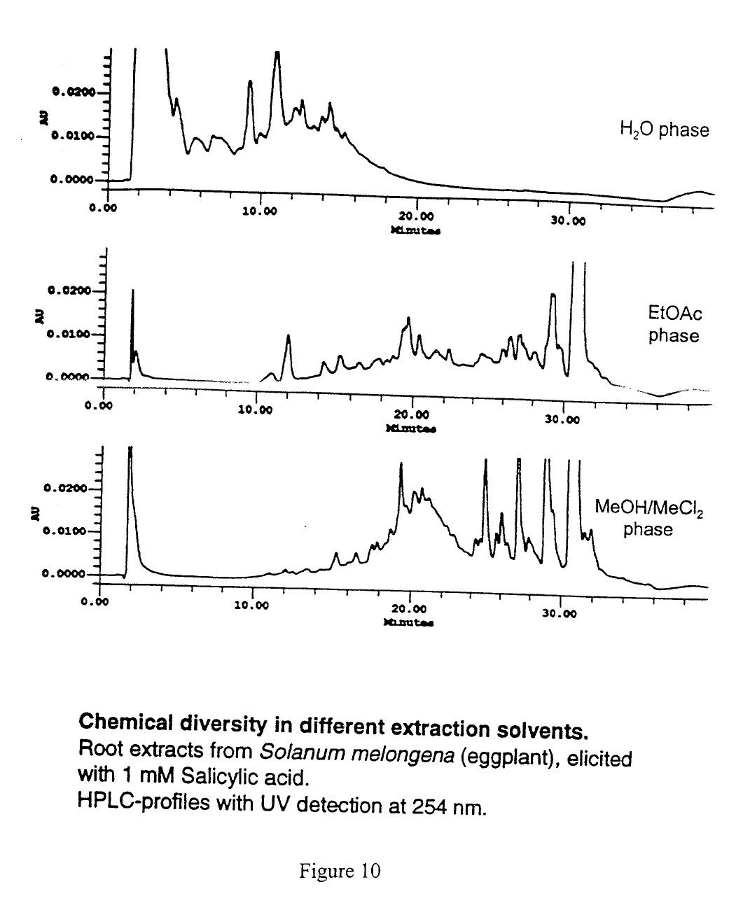

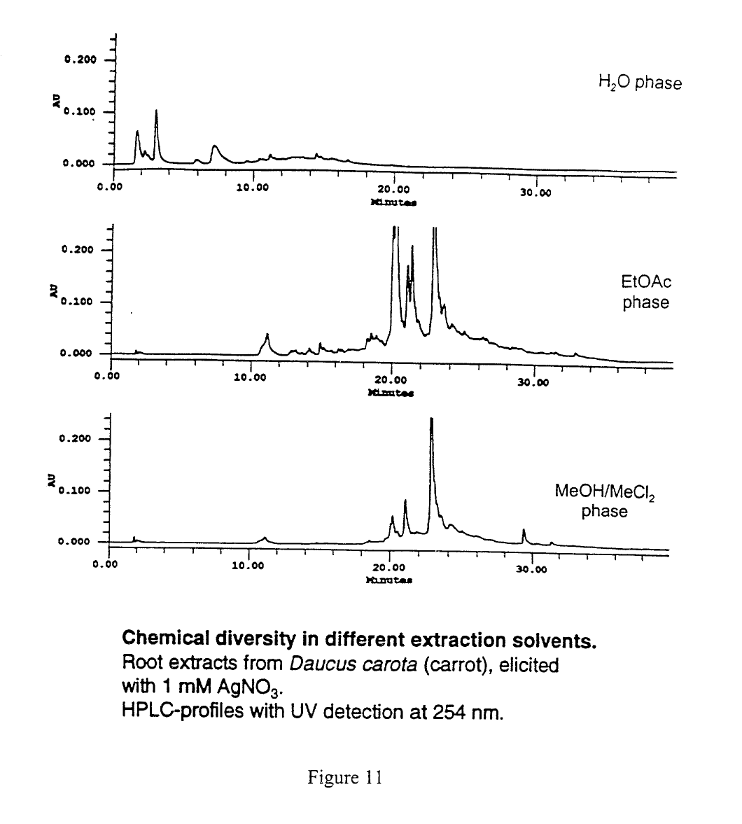

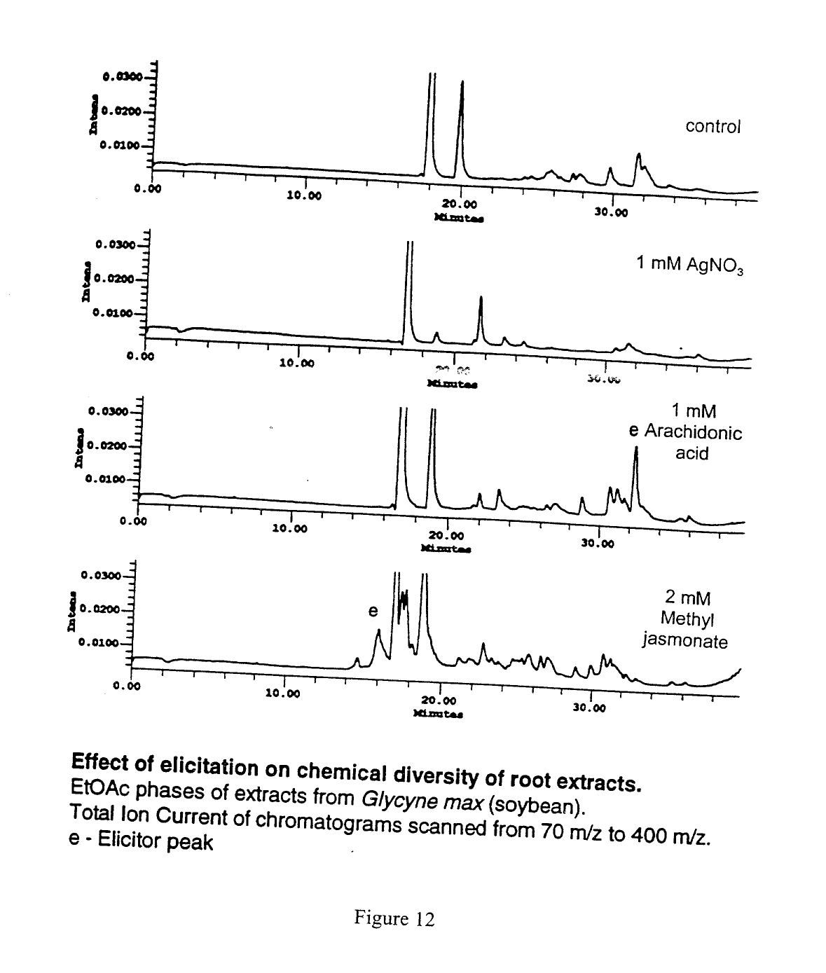

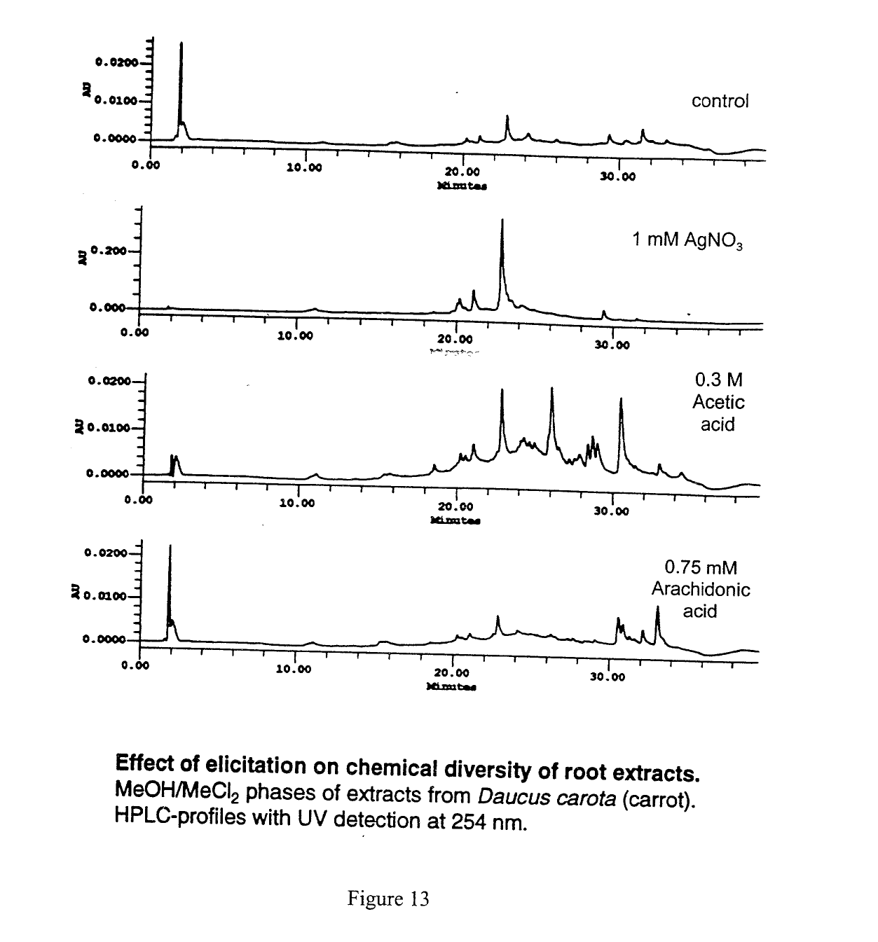

FIGS. 5, 6, 7 and 8 are HPLC profiles of the diversity of compounds exuded or leached from the roots of Brassica juncea, Datura metel, Lupinus polyphyllus and Melilotus medicaginoides, respectively, treated with different elicitors. UV detection was at 254 nm. No compound was identified.

### Example 4

### Root Extraction

The complete root systems of all plants from each tray were excised, drained and weighed. Up to 30 g of the root systems (fresh weight) were sampled and stored at −20° C. The root tissue was homogenized in a laboratory blender (Model 31BL91, Waring, New Hartford, Conn.) in 2 volumes H₂O for approximately 30 sec. The homogenate was transferred to a 150 mL Corex tube (Coming, Inc., Coming, N.Y.) and a two-phase extraction was carried out by adding 2 volumes of ethyl acetate (EA) and shaking the sealed tube for 30 min at 200 rpm (Shaker Model PR70, Hoefer Scientific Instrument, San Francisco, Calif.). The tubes were then centrifuged (Model Avanti J-25, Rotor No. JA-14, Beckman Instrument Inc., Palo Alto, Calif.) for 10 min at 4000×g, in order to form a clear EA layer in the upper phase. The two-phase extraction was repeated with an additional single volume of EA. Following two extractions, the EA extracts were combined and placed in the fume hood until the EA volume was reduced to approximately half.

The EA extract was divided into disposable glass tubes in proportion to the weight of the extracted roots, so that each tube contained the extracts equivalent to at least 5 g root tissue. The EA extract was evaporated in a speed vac (Model AES2010, Savant Instruments, Inc., Farmingdale, N.Y.), the tubes were sealed and stored at 20° C. The H₂O phase, containing the root tissue and some EA residues, was filtered, pressed through a 70 μm nylon mesh (Spectra/Mesh Nylon Filters, Spectrum, Houston, Tex.) and placed in a 125 mL separatory funnel until the lower water phase separated from the upper layer (approximately 30 min). The water layer was decanted into 50 mL polypropylene disposable tubes and centrifuged for 30 min at 4000×g (Rotor No. JS-4.0, Beckman Instrument Inc.). The supernatant was divided into 60 mL glass bottles in proportion to the weight of the extracted root tissue (extract equivalent of 5 g of root tissue per bottle), freeze dried overnight (Genesis SQ12, Virtis, Gardiner, N.Y.) and stored at −20° C.

The remaining root tissue was further extracted with 2 volumes methanol (MeOH)/CH₂Cl₂ (1:3), shaken for 30 min at 200 rpm, filtered and pressed through 70 μm fluorocarbon mesh (Spectra/Mesh Fluorocarbon Filters, Spectrum). The filtrate was transferred to a separatory funnel until the lower MeOH/CH₂Cl₂ phase became clear (up to 30 min). The MeOH/CH₂Cl₂ extract was then divided into disposable glass tubes (equivalent of 5 g root tissue per tube), dried in a speed vac and stored in a similar manner to the EA extracts.

### Example 5

### High-pressure Liquid Chromatography (HPLC) Analysis of Extracted Products

The chromatography separation of extracted products was performed with an HPLC-system consisting of Waters 996 Photodiode Array Detector (PDA) with usable UV range from 190 to 800 nm; a Waters 717 plus autosampler; two Beckman 110B solvent Delivery Modules, connected with a Beckman System Organizer (mixer) and a Beckman System Gold Analog Interface Module 406. The Beckman solvent delivery system was controlled by a NEC PC-8300 computer. Chromatography and spectral data was managed by Waters Millennium chromatography software, version 2.10, using a NEC image 466es computer. All hardware components, except the solvent delivery system, were connected through a standard IEEE communication system. Compounds were separated on a Waters Nova Pak® C-18 reverse phase column, 3.9×150 mm, 60 Å pore size, and 4 μm particle size.

The mobile phase consisted of two components: Solvent A-0.5% ACS grade acetic acid in double distilled water, pH 3-3.5; and Solvent B-acetonitrile. Prior to use, each batch of solvent A was degassed under vacuum and ultrasonication for 5 minutes.

The mobile phase flow was adjusted to 1 mL/min, and a gradient mode of separation was used for all separations. The gradient profile was as follows:

0-20min 0% B-100% B;

20-22 min 100% B;

22-25 min 100% B-0% B;

25-33 min 0% B (column equilibration for next injection.

Compounds were detected with PDA detector within the wavelength range of 200 to 400 nm or with Waters Thermabeam™ Mass Detector. The column temperature was ambient.

All plants were grown hydroponically and treated with an elicitor, as described in Example 1. The roots were harvested and subjected to an extraction procedure as described in Example 4. The accompanying drawings are HPLC profiles (obtained as in Example 5) of chemicals recovered from the extracts, which extracts are recovered from roots harvested from the plants treated with elicitors described in the drawings.

### Example 6

### Bioassay of Leaf and Root Exudates

A. Preparation of cuticular washings: Leaves from plants were contacted with 5 mL of solvent (methylene chloride) contained in plastic sandwich bags (quart size, 7 in×8 in) as containers. To standardize the cuticular wash concentration by relating it to the used leaf surface from which it was taken, approximately 60 cm² of leaf surface were used. Sandwich bags with zippers were used to guarantee that the surface of leaf was totally moisturized with solvent. To facilitate the removal of cuticular compounds the bag containing a leaf and the solvent was shaken for approximately 20-40 seconds. The end of bag was cut and the content removed into 20 mL scintillation vials, and closed with Teflon or foil faced liner screw caps and stored in refrigerator. Alternatively, the solvent containing the cuticular washings can be dried inside the scintillation vial before cold storage.

B. Preparation of bacteria and fungus suspensions: six different organisms were used for antibacterial and antifungal screening: 1) Escherichia coli K-12. F, prototropic Str.; 2) Staphylococcus aureus subsp. Aureus; 3) Pseudomonas aeruginosa; 4) Saccharomyces cerevisiae; 5) Aspergillus flavus; and 6) Penicillium nigra. Bacteria (Escherichia coli, Staphylococcus aureus, and Pseudomonas aeruginosa) were maintained on solid agar media (L B Agar, Miller). Before screening, bacteria was transferred into liquid media and cultivated for 12 hours at 37° C. on shaker with a cell density 105-6. Saccharomyces cerevisiae (yeast), Aspergillus flavus and Penicillium nigra were cultivated on potato dextrose media. Before treatment yeast cells were transferred into liquid media and cultivated for 48 hours at 30° C. on a shaker. The spores of Aspergillus flavus were washed with distilled water from fungus surface grown in Petri dish and resuspended in fresh distilled water.

The standard method used to determine in vitro antibacterial and antifungal activity of leaf surface cuticular washings consisted of testing suspension of microorganisms and spores of fungus for growth inhibition in the presence of washings. The antibacterial and antifungal activity was indicated by 30% or more, growth reduction of cells/spores in the presence of cuticular washings. All samples were plated in 3 replicates. One mL of nutrient media was put in each well in 24 well plates. Thereafter, 10 μL of methylene chloride solution containing cuticular washings were placed on agar surface and after the drop dried (2-3 min) 30 μL of microorganism suspension or fungus spores were plated on top of agar and equally distributed throughout the surface. After 24 hours of incubation at +30° C., the plates were examined for the presence/absence of activity. To test antimicrobial/antifungal activity of root exudates, cell suspension was plated and spread on the agar surface into each of 24 well plates. Using a 5 mL Eppendorf pipet tip attached to a vacuum line, a hole was made in the center of each well and 20 μL of exudate dissolved in water (5 mg/300 mL) gently poured into the hole. The following elicitors were employed: methyl salicylate, methyl jasmonate, silver nitrate, acetic acid, and chitosan.

To harness the vast and largely unexplored diversity of biological natural products exuded by plant roots, an efficient method for collecting root exudates from various plants was developed. The method was based on a modified hydroponic technology, which allowed maintaining plant roots in water or diluted nutrient solution followed by analysis of compounds exuded from roots.

The seeds of cultivated and wild species obtained from the commercial seed companies or botanical gardens were germinated in a greenhouse inside a 0.9 cm in diameter, 0.5 cm deep well cut into Grodan rockwool cubes (3.4 cm width×3.4 cm depth×3.7 cm height). Rockwool cubes were placed inside standard greenhouse plastic trays (dimensions 52 cm width×25 cm depth×7 cm height) and watered with an overhead misting system. Seeds were allowed to germinate for 3-6 days until the roots started to emerge from the bottom of the rockwool cube.

After germination, the cubes containing the seedlings were inserted into the center of a Styrofoam ring with an inside diameter 3.2 cm, outside diameter 8.2 cm and 2.5 cm thickness. The ring was floated on the surface of 400-800 mL of hydroponic nutrient solution (2 g/L Hydro-Sol [Scotts-Sierra Horticultural products Comp., Marysville, Ohio.] supplemented with 1.2 g/L Ca[NO₃]₂) contained inside a light impermeable, high density polyethylene cylinder (9.0 cm in diameter, 16 cm in height).

Aeration was provided by shaking the cylinders at 50 rpm on a platform shaker (Labline Orbital Shaker, Model 3590). Seedlings were cultivated hydroponically in this system for 3 to 6 weeks with roots growing in a nutrient solution. Thereafter, the root system (average root dry weight 0.1±0.05 g) was removed from the nutrient solution and placed inside a 30 mL glass beaker, containing 10-20 mL of distilled water or distilled water supplemented with an elicitor. To prevent water loss from the plant canopy and drying of the collecting solution, plant shoots were covered with transparent plastic bags. After 24 hours, unless noted otherwise, a small sample from the root solution was removed and analyzed for the phytosecreted products. This system of hydroponic plant cultivation and exudate collection is referred to as the standard exudate collection system. Root exudates may also be freeze-dried and stored in the freezer at −20° C. When needed, the exudate powder may be re-dissolved in water and used for screening or chemical analysis.

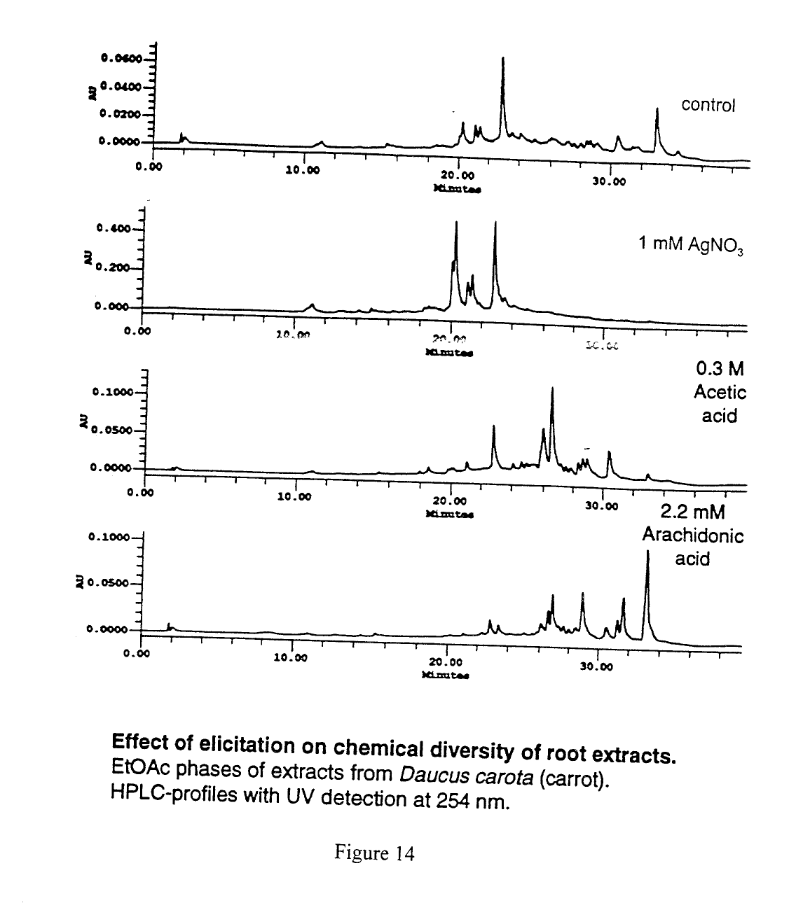

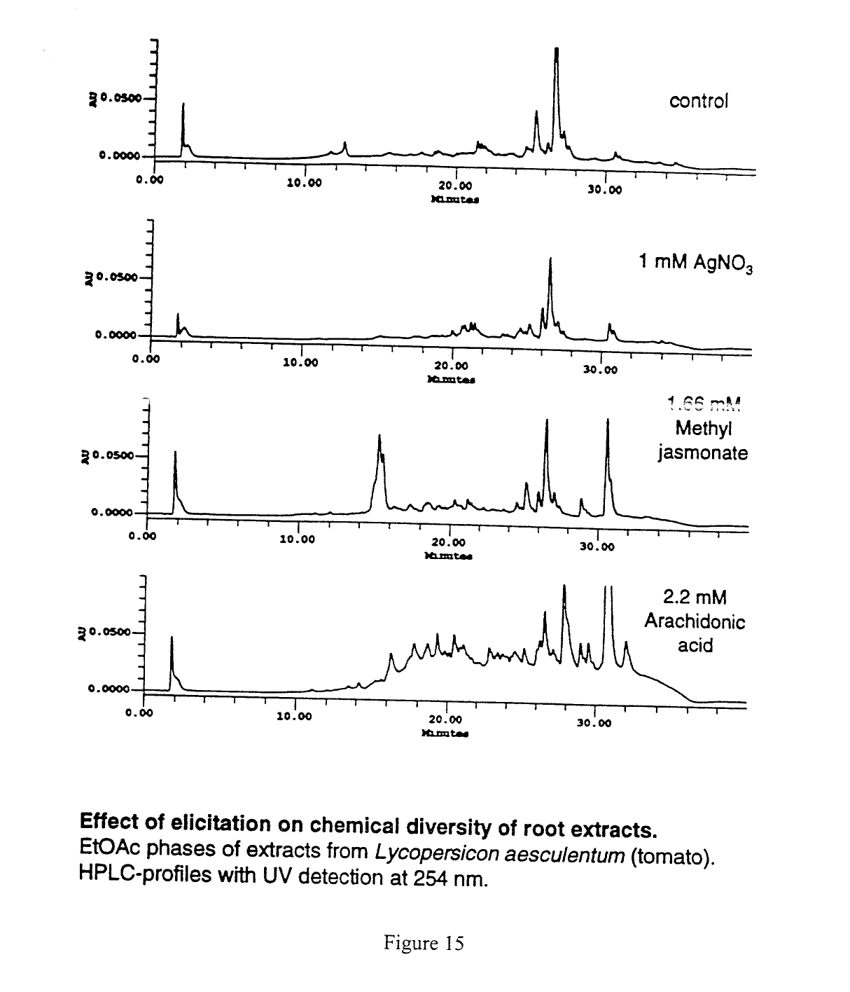

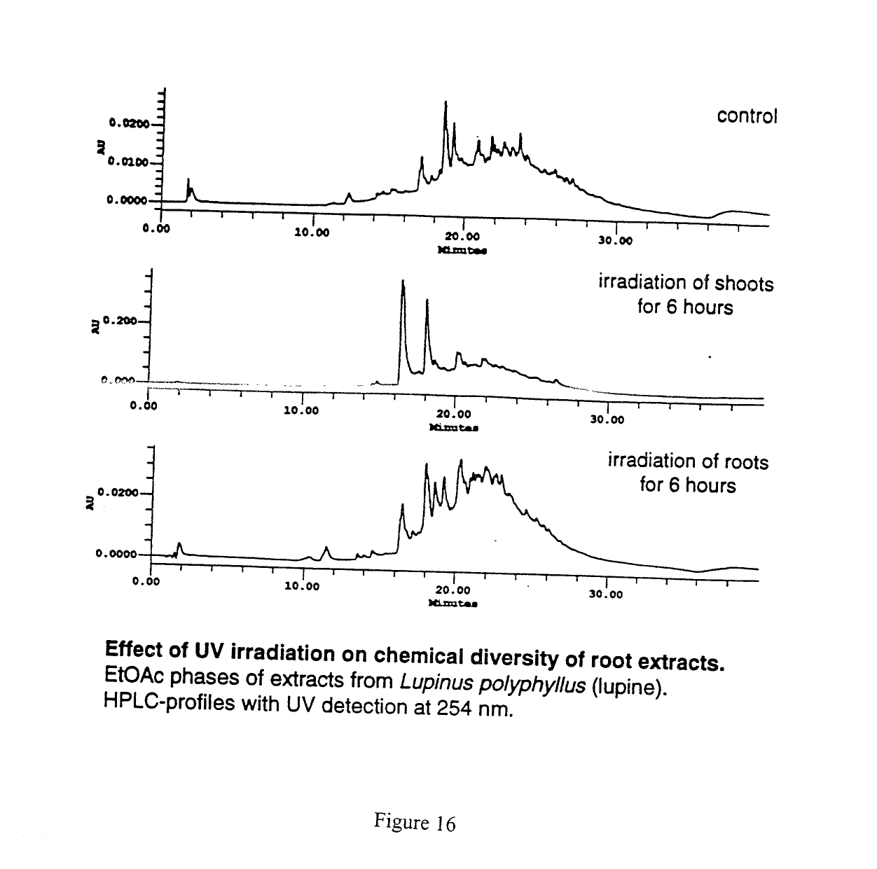

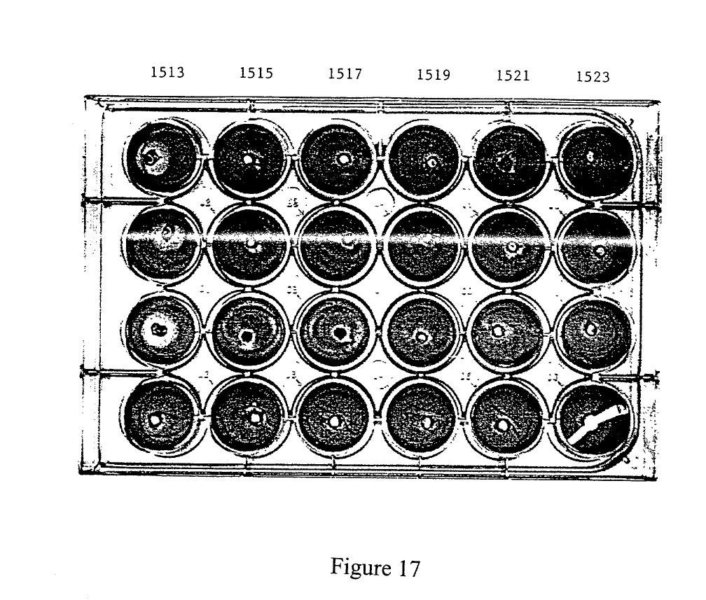

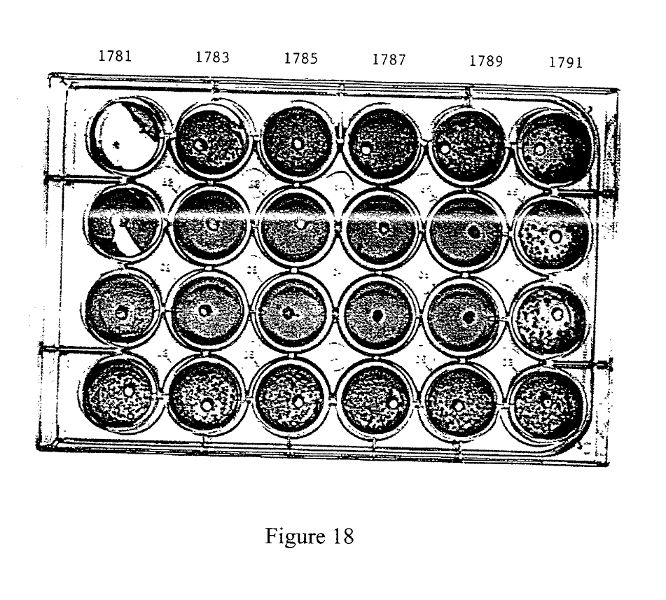

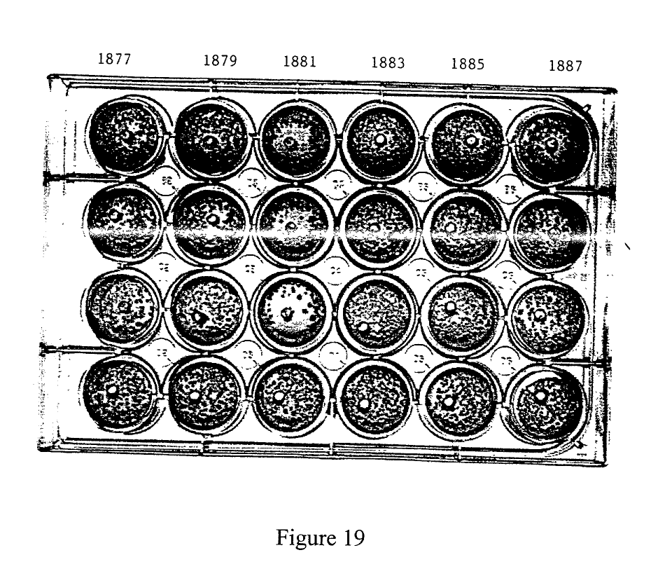

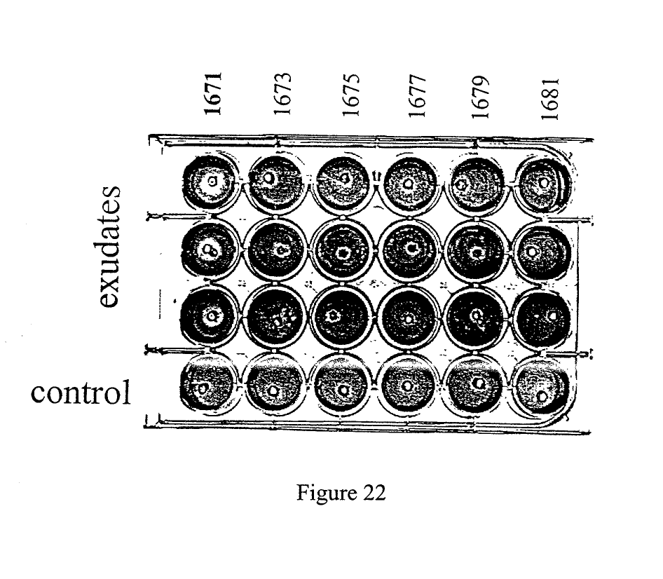

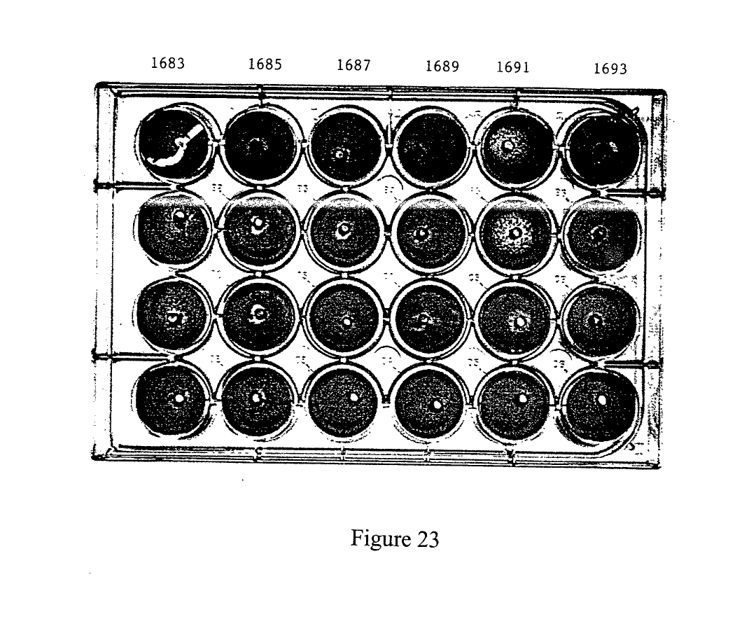

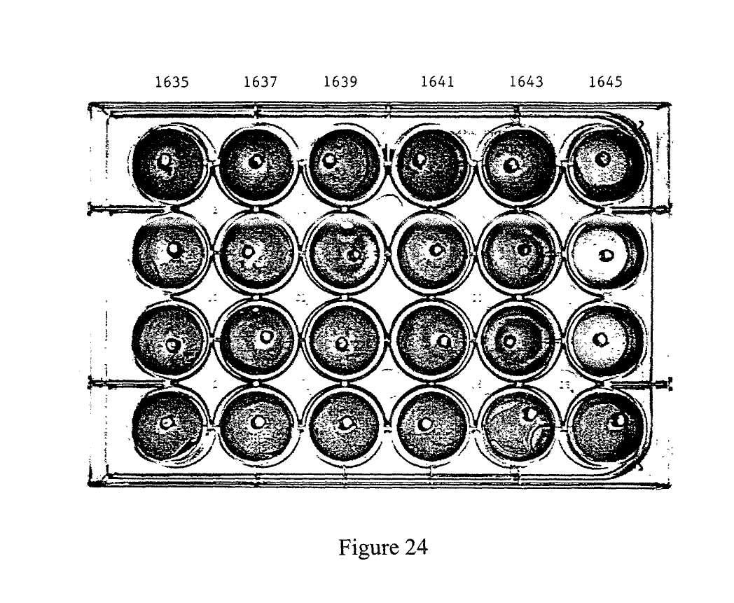

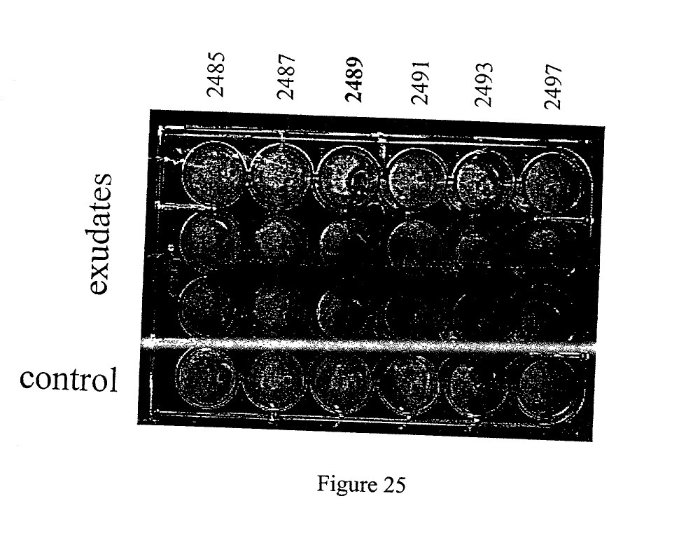

A total of 844 plant species root exudates elicited were prepared and tested against six-above mentioned microbial/fungal cultures (Table 2). The final concentration of exudates used in assay was 5 mg of dry exudate diluted in 300 μL of distilled water. It has been found that great majority of material tested at this concentration did not affect adversely growth of the tested organisms. FIGS. 14, 15, 16, 19, 20, 21, and 22 demonstrate the results of the inhibition of the biological activity. A number of primary hits has been identified against all, but one (Aspergillus) microorganisms tested. The hit rate under the conditions used varied between 0% and 7.8% (Table 1). It is noteworthy that majority of the hits come from exudates from elicited roots. The unusually high proportion of hits in the materials elicited by silver may also be partially explained by the toxic effects of silver on a given microorganism.

TABLE 1
Frequency of Antimicrobial Effects of Root Exudates

| Target Organism | Number of Hits | Hit Rate (%) |
|---|---|---|
| Escherichia coli | 23 (884)¹ | 2.6 |
| Staphylococcus aureus | 34 (884) | 3.8 |
| Pseudomonas aeruginosa | 8 (102) | 7.8 |
| Aspergillus flavus | 0 (510) | 0 |
| Penicillium nigra | 4 (102) | 3.9 |
| Saccharomyces cerevisiae | 6 (718) | 0.8 |

¹Number in parenthesis indicate total number of root exudate samples tested for a particular microorganism.

The list of plant species from which root exudates show antimicrobial/antifungal activity are shown below in Table 2. The strengths of the activity is denoted by the number of “*”, with a larger number of “*” referring to greater activity. As used in the table, the following abbreviations are intended to represent:

A.F. Aspergillus flavus

E.c.—Escherichia coli

S.a.—Staphylococcus aureus

S.c.—Saccharomyces cerevisiae

P.n.—Penicillium nigra

P.a.—Pseudomonas aeruginosa

Acetic a.—acetic acid

TABLE 2
Root Exudates Showing Antimicrobial/Antifungal Activity

| | I.D. # | Plant Name | Elicitor | A.f | E.c. | S.a. | S.c. | P.n. | P.a |
|---|---|---|---|---|---|---|---|---|---|
| 1 | 845 | Atropa belladonna | Silver | | | * | | | |
| 2 | 857 | Erythrina glabelliformis | Silver | | | * | | | |
| 3 | 949 | Ipomoea tricolor | Silver | | | * | | | |
| 4 | 1363 | Erythrina galli | Silver | | * | * | | | |
| 5 | 1475 | Celosia cristata | Acetic a. | | * | * | | | |
| 6 | 1501 | Gallium spurium | Acetic a. | | | * | | | |
| 7 | 1513 | Laurus nobilis | Silver | | * | * | | | |
| 8 | 1563 | Vitis labrusca | Control | | * | | | | |
| 9 | 1585 | Gratiola officinalis | Control | | | * | | | |
| 10 | 1617 | Symphytum officinalis | Silver | | * | | | | |
| 11 | 1645 | Hosta fortunea | Acetic a. | | | | * | | |
| 12 | 1649 | Cassia hebecarpa | Control | | | * | | | |
| 13 | 1659 | Thalictrum flavum | Acetic a. | | * | * | | | |
| 14 | 1671 | Scutellaria altissima | Silver | | * | * | | | |
| 15 | 1681 | Portulacca oleracea | Silver | | | | * | | |
| 16 | 1683 | Portulacca oleracca | Chitosan | | | | * | | |
| 17 | 1685 | Portulacca oleracea | Meta | | | | * | | |
| 18 | 1691 | Scutellaria creticola | Silver | | | * | | | |
| 19 | 1695 | Physalis ixocarpa | Silver | | | * | | | |
| 20 | 1757 | Geum fourieri | Control | | | | * | | |
| 21 | 1791 | Gentiana tibetica | | | * | * | | | |
| 22 | 1875 | Linum hirsutum | Acetic a. | | | | * | | |
| 23 | 1879 | Aconitum napellus | Acetic a. | | | | | | |
| 24 | 1881 | Aconitum napellus | Silver | | * | * | | | |
| 25 | 1887 | Podophyllum aunodii | Silver | | | | | | |
| 26 | 1897 | Thymus cretaceus | Silver | | | | | * | |
| 27 | 1913 | Hosta fortunea | Silver | | *** | *** | | * | |
| 28 | 1975 | Hosta fortunea | Chitosan | | ** | *** | | | |
| 29 | 1985 | Carlina acaulis | Silver | | * | *** | | | |
| 30 | 2003 | Chamaechrista fasciculata | Silver | | ** | *** | | * | |
| 31 | 2013 | Pinus pinea | Silver | | ** | *** | | ** | |
| 32 | 2043 | Peganum harmala | Silver | | | * | | | |
| 33 | 2053 | Tamarindus indica | Silver | | * | *** | | | |
| 34 | 2063 | Carica papaya | Silver | | * | *** | | | |
| 35 | 2111 | Cistus incanus | Control | | *** | ** | | | |
| 36 | 2161 | Capparis inermis | Silver | | *** | *** | | | *** |
| 37 | 2177 | Cupressus lusitanica | Silver | | ** | *** | | | *** |
| 38 | 2133 | Diopiros khaki | Acetic a. | | | ** | | | |
| 39 | 2135 | Diopiros khaki | Silver | | ** | *** | | | *** |
| 40 | 2137 | Diopiros khaki | Chitosan | | | ** | | | |
| 41 | 2145 | Eryngium campestre | Silver | | ** | ** | | | *** |
| 42 | 2255 | Aesculus woerlitzienis | Silver | | *** | *** | | | *** |
| 43 | 2265 | Aesculus hippocastanum | Silver | | *** | *** | | | *** |
| 44 | 2299 | Cupresstis sempervirens | Silver | | | *** | | | |
| 45 | 2315 | Celtis occidentalis | Silver | | ** | *** | | | |
| 46 | 2325 | Calycanthus floridus | Silver | | ** | ** | | | |
| 47 | 2335 | Chinionanthus praecox | Control | | ** | ** | | | |
| 48 | 2345 | Clematis manschurica | Silver | | ** | ** | | | |
| 49 | 2377 | Liatris spicata | Silver | | | * | | | |
| 50 | 2379 | Liatris spicata | Silver | | | * | | | |
| 51 | 2407 | Cladium mariscus | Silver | | ** | *** | | | |
| 52 | 2425 | Lablab purpureus | Silver | | | ** | | | |
| 53 | 2455 | Campanula carpatica | Silver | | | * | | | |
| 54 | 2443 | Chilopsis linearis | Silver | | | * | | | |
| 55 | 2461 | Thuja occidentalis | Silver | | ** | ** | | | |
| 56 | 2479 | Cosmos sulphureus | Silver | | ** | ** | ** | | |
| 57 | 2489 | Cunningamia lanceolata | Silver | | ** | ** | ** | | |
| 58 | 2503 | Euptelea pleiosperma | Silver | | | ** | | | |
| 59 | 2535 | Juglans regia | Acetic a. | | ** | ** | | | |

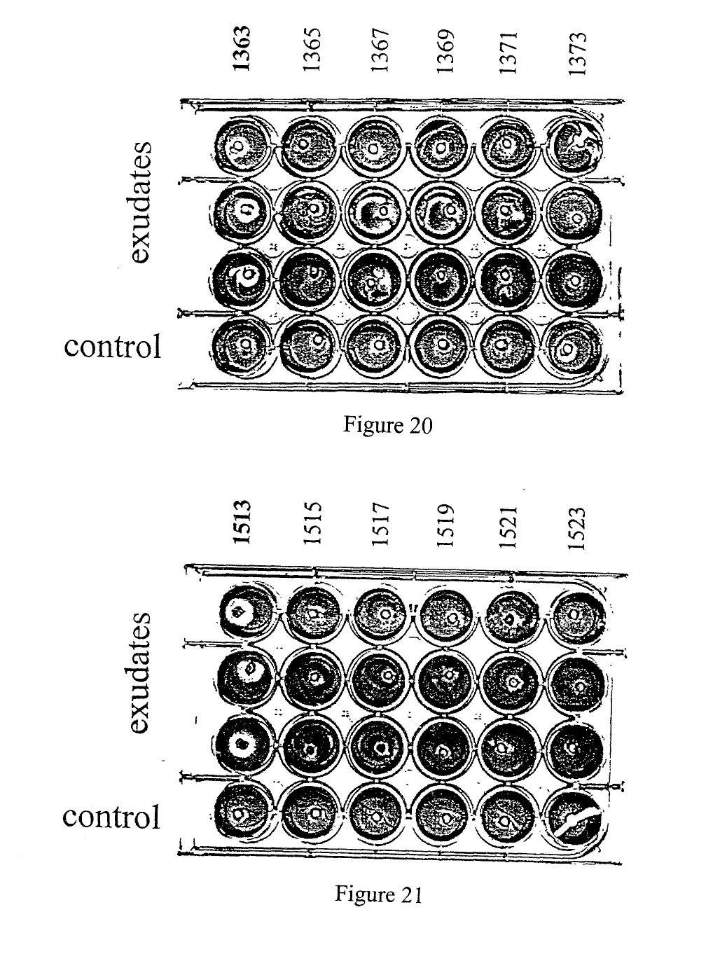

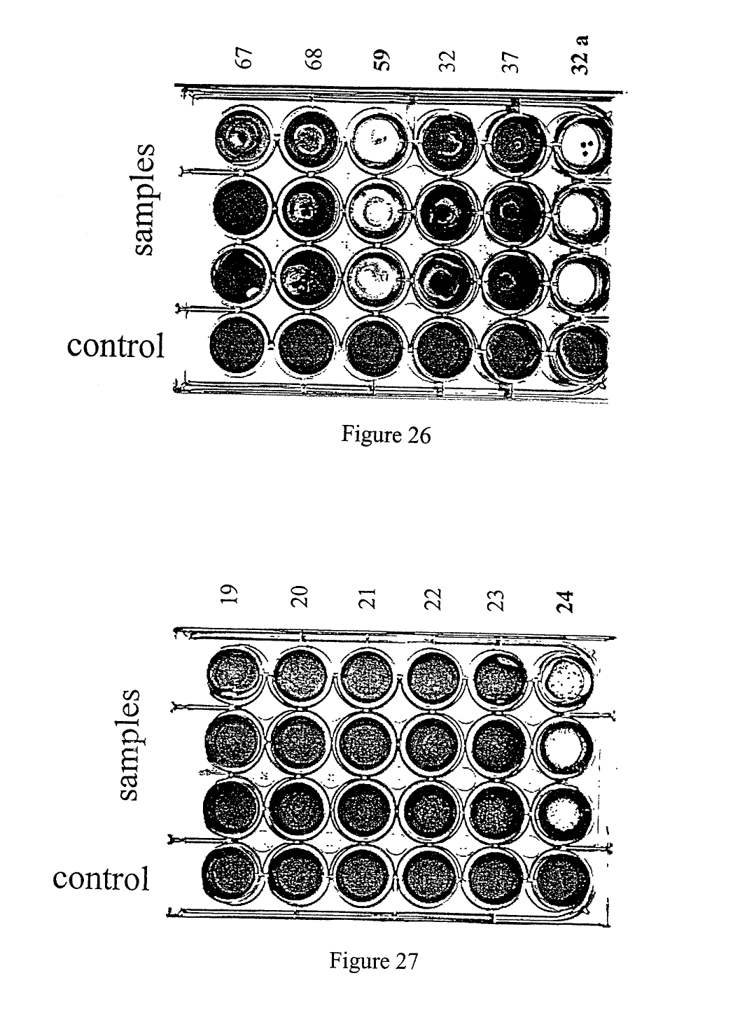

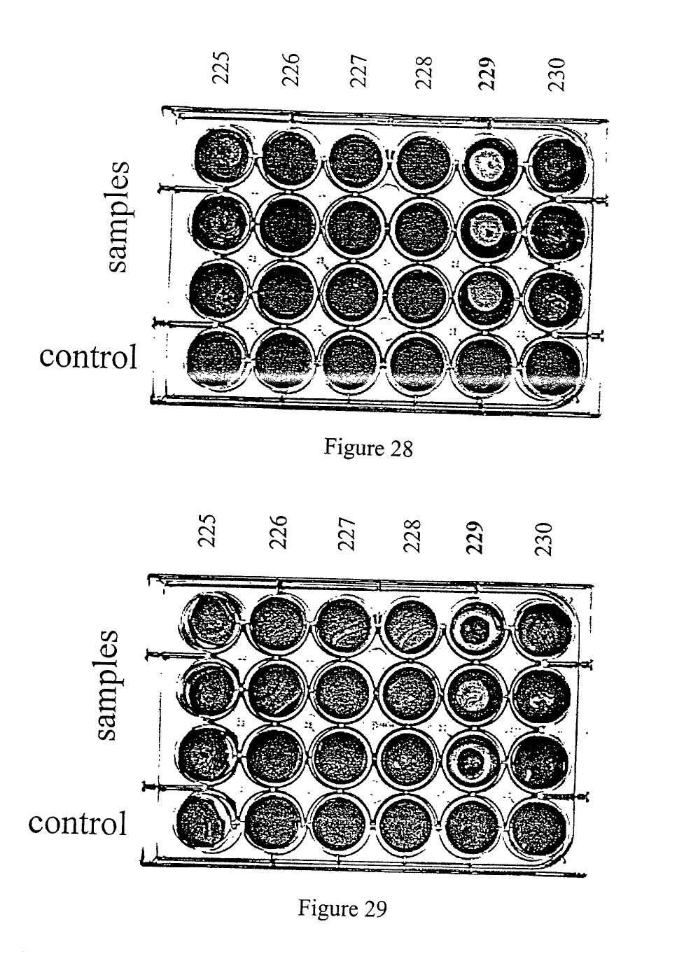

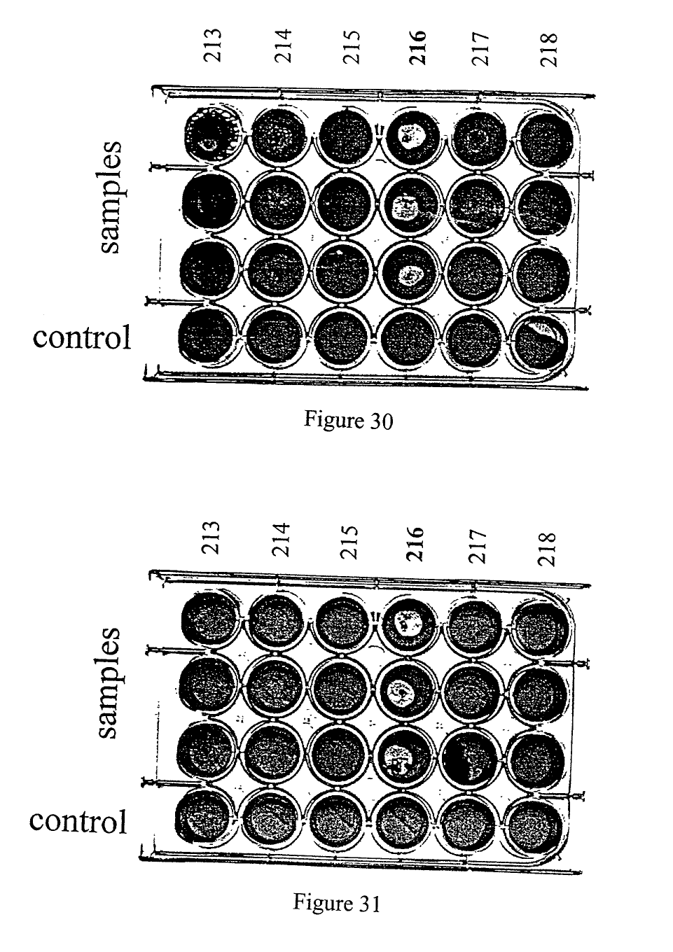

A total of 400 plant species cuticular washings has been prepared and tested against six above mentioned microbial/fungal cultures and the amount of cuticular washings used for each well was 10 82 L. It has been found that a majority of materials tested at this concentration did not affect adversely growth of the tested organisms. However, a significant percentage of cuticular washings showed antimicrobial activity. FIGS. 20, 21, 26, 27, 28, 29, 30, and 31 demonstrate the results of this activity. A number of strong antimicrobial hits have been identified for Escherichia coli, Staphylococcus aureus and Saccharomyces cerevisiae Table 3. The hit rate among cuticular washings from different plant species varies from 0.5 to 5.0% depending on the microorganism.

TABLE 3
Plant Species of which Leaf Surface Cuticular Washings Show Strong
Antifungal or Antimicrobial Activity

| | Sample | | | | |
|---|---|---|---|---|---|
| | Identification | Plant name | S.a. | E.c. | S.c. |
| 1 | 125 | Taxodium distichum | x | | |
| 2 | 133 | Grevillea robusta | x | | |
| 3 | 136 | Betula pendula | x | | |
| 4 | 171 | Anthurium elegans | x | | |
| 5 | 198 | Foenix zeulinica | | x | x |
| 6 | 216 | Oreopanax capitate | x | x | |
| 7 | 229 | Eucalyptus rudis | x | x | x |
| 8 | 248 | Betula nigra | x | | |
| 9 | 274 | Paeonia dahurica | | | |
| 10 | 276 | Betula alba | x | x | |
| 11 | 294 | Thalictrum sp. | x | x | |
| 12 | 302 | Agrimonia eupatori | x | | |
| 13 | 355 | Salix babilinics | x | | |
| 14 | 377 | Cerasus janonica | | | |

### Example 7

### Sniffing Test of Root Exudates and Cuticular Washings

The assay was done by thawing a frozen sample to room temperature, opening a glass vial containing a sample, sniffing it, and immediately marking the results. Samples are stored frozen −20° C. in tightly sealed glass vials (5 mL vials for cuticular washings and 20 mL vials root exudates). The amount of root exudates in each vial ranges from 5 to 500 mg. The amounts of cuticular compounds in each vial ranges from 5 to 100 mg.

As demonstrated by Tables 4 and 5, a significant proportion of samples have a strong fragrance. There are 36 out of 100 samples of exudates and 20 out of 100 tested cuticular washing with strong fragrance. Root exudates were treated with Acetate 03-0.1% acetic acid, AgNO3 02-0.5 mM Ag(NO₃)₂, Chito 02-0.1% chitosan, water, HSL 01-200 μM N-hexanoyl homoerinelactone, and MeJa 03-100 μM methyl jasmonate. In Table 4 and 5, smell was rated by the scientist as follows: 0 is no smell; 1 is light smell; 2 is medium smell; and 3 is strong smell.

TABLE 4
Sniffing Test on Root Exudates

| Sample | Rating | Family | Genus | Species | Treatment |
|---|---|---|---|---|---|
| 1199 | 3 | Solanaceae | Hyoscyamus | niger | Acetate 03 |
| 1201 | 3 | Solanaceae | Hyoscyamus | niger | AgN03 02 |
| 1203 | 2 | Solanaceae | Hyoscyamus | niger | Chito 02 |
| 1205 | 3 | Fabaceae | Genista | tinctoria | Acetate 3 |
| 1207 | 2 | Fabaceae | Genista | tinctoria | AgN03 02 |
| 1209 | 2 | Fabaceae | Cicer | arietinum | control 01 |
| 1211 | 3 | Fabaceae | Cicer | arietinum | Acetate 03 |
| 1213 | 3 | Fabaceae | Cicer | arietinum | AgN03 02 |
| 1215 | 3 | Fabaceae | Cicer | arietinum | Chito 02 |
| 1217 | 1 | Fabaceae | Cicer | arietinum | HSL 01 |
| 1219 | 2 | Fabaceae | Thermopsis | fabacea | HSL 01 |
| 1221 | 3 | Fabaceae | Thermopsis | fabacea | acetate 03 |
| 1223 | 2 | Cucurbitaceae | Trichosanthes | kirilowii | control 01 |
| 1225 | 3 | Cucurbitaceae | Trichosanthes | kirilowii | acetate 03 |
| 1227 | 1 | Cucurbitaceae | Trichosanthes | kirilowii | HSL 01 |
| 1229 | 2 | Cucurbitaceae | Trichosanthes | kirilown | Chito 02 |
| 1233 | 0 | Asteraceae | Xanthium | sibiricum | HSL 01 |
| 1235 | 3 | Solanaceae | Brugmansia | suaevolens | AgN03 02 |
| 1237 | 0 | Solanaceae | Brugmansia | suaevolens | HSLOI |
| 1239 | 1 | Asteraceae | Eclipta | alba | control 01 |
| 1241 | 3 | Asteraceae | Eclipta | alba | Acetate 03 |
| 1243 | 2 | Asteraceae | Ectipta | alba | AgN03 02 |
| 1245 | 0 | Asteraceae | Eclipta | alba | Chito |
| 1247 | 3 | Asteraceae | Aremisia | absinthium | control 01 |
| 1249 | 3 | Asteraceae | Artemisia | absinthium | Acetate 3 |
| 1251 | 2 | Asteraceae | Artemisia | absinthium | AgN03 02 |
| 1253 | 0 | Asteraceae | Artemisia | absinthium | Chito 02 |
| 1255 | 1 | Asteraceae | Silybum | marianum | control 01 |
| 1257 | 2 | Asteraceae | Silybum | marianum | Acetate 03 |
| 1259 | 3 | Asteraceae | Silybum | marianum | AgN03 02 |
| 1261 | 3 | Asteraceae | Silybum | marianum | Chito 02 |
| 1263 | 2 | Asteraceae | Silybum | marianum | MeJa 03 |
| 1267 | 3 | Apiaceae | Cnidium | monnieri | Acetate 02 |
| 1269 | 2 | Apiaceae | Cnidium | monnieri | AgN03 02 |
| 1271 | 2 | Apiaceae | Cnidium | monnieri | Chito 02 |
| 1275 | 2 | Apiaceae | Cnidium | monnieri | HSL 01 |
| 1277 | 3 | Solanaceae | Brugmansia | suaevolens | control 01 |
| 1279 | 2 | Solanaceae | Brugmansia | suaevolens | Acetate 03 |
| 1281 | 1 | Clusiaceae | Hypericum | perforatum | control 01 |
| 1283 | 2 | Clusiaceae | Hypericum | perforatum | Acetate 03 |
| 1285 | 1 | Clusiaceae | Hypericum | perforatum | AgN03 02 |
| 1287 | 1 | Clusiaceae | Hypericum | perforatum | Chito 02 |
| 1289 | 3 | Clusiaceae | Hypericum | perforatum | MeJa 03 |
| 1291 | 3 | Boraginaceae | Anchusa | officinalis | control 01 |
| 1293 | 2 | Boraginaceae | Anchusa | officinalis | Acetate 03 |
| 1297 | 3 | Asteraceae | Xanthium | sibiricum | AgNO3 02 |
| 1299 | 3 | Zygophilaceae | Larrea | tridentata | control 01 |
| 1301 | 3 | Zygophyllaceae | Larrea | tridenta | Acetate 03 |
| 1303 | 2 | Zygophyllaceae | Larrea | tridenta | AgN03 02 |
| 1305 | 0 | Zygophyllaceae | Larrea | tridenta | Chito 02 |
| 1307 | 2 | Zygophyllaceae | Larrea | tridenta | MeJa 03 |
| 1309 | 2 | Lamiaceae | Scutellaria | baicalensis | control 01 |
| 1311 | 2 | Lamiaceae | Scutellaria | baicalensis | Acetate 03 |
| 1313 | 2 | Lamiaceae | Scutellaria | baicalensis | AgN03 02 |
| 1315 | 0 | Lamiaceae | Scutellaria | baicalensis | Chito 02 |
| 1317 | 3 | Fabaceae | CytiSSLIS | scopartus | control 01 |
| 1319 | 1 | Fabaceae | Cytissus | scoparius | Acetate 03 |
| 1321 | 3 | Apocynaceae | Rauvolfia | caffra | Chito 02 |
| 13323 | 1 | Cyperaceae | Cyperus | esculentus | control 01 |
| 1325 | 3 | Cyperaceae | Cyperus | esculentus | Acetate 03 |
| 1327 | 2 | Cyperaceae | Cyperus | esculentus | AgN03 02 |
| 1329 | 2 | Cyperaceae | Cyperus | esculentus | Chito 02 |
| 1331 | 0 | Asteraceae | Amica | chamissois | HSL 01 |
| 1333 | 3 | Solanaceae | Physalis | ixocarpa | control 01 |
| 1335 | 3 | Solanaceae | Physalis | ixocarpa | Acetate 03 |
| 1337 | 2 | Solanaceae | Physalis | ixocarpa | AgN03 02 |
| 1339 | 0 | Solanaceae | Physalis | ixocarpa | Chito 02 |
| 1341 | 1 | Solanaceae | Physalis | ixocarpa | MeJa 03 |
| 1343 | 3 | Apiaceae | Angelica | polymorha-sinesis | control 01 |
| 1345 | 2 | Apiaceae | Angelica | polymorha-sinesis | AgNO3 02 |
| 1347 | 3 | Apiaceae | Angelica | polymorha-sinesis | AgN03 02 |
| 1349 | 2 | Apiaceae | Angelica | polymorha-sinesis | Chito 02 |
| 1351 | 1 | Apiaceae | Angelica | polymorha-sinesis | Meja 03 |
| 1353 | 2 | Rosaceae | Agrimonia | pilosa | control 01 |
| 1357 | 3 | Asteraceae | Arnica | chamissois | Acetate 03 |
| 1359 | 0 | Fabaceae | Erythrina | christa-calli | control 01 |
| 1361 | 3 | Fabaceae | Erythrina | christa-galli | Acetate 03 |
| 1363 | 2 | Fabaceae | Erythrina | christa-cralli | AgNO3 02 |
| 1365 | 1 | Fabaceae | Erythrina | christa-galli | Chito 02 |
| 1367 | 0 | Fabaceae | Erythrina | christa-galli | HSL 01 |
| 1369 | 3 | Ranunculaceae | Aquilegia | vulgaris | control 01 |
| 1371 | 1 | Ranunculaceae | Aquilegia | vulgaris | Acetate 03 |
| 1373 | 1 | Ranunculaceae | Aquilegia | vulgaris | AgNO3 02 |
| 1375 | 1 | Ranunculaceae | Aquilegia | vulgaris | Chito 02 |
| 1377 | 2 | Ranunculaceae | Aquilegia | vulgaris | MeJa 03 |
| 1379 | 3 | Lamiaceae | Leonurus | sibiricus | control 01 |
| 1381 | 3 | Lamiaceae | Leonurus | sibiricus | Acetate 03 |
| 1383 | 2 | Lamiaceae | Leonurus | sibiricus | AgN03 02 |
| 1385 | 0 | Lamiaceae | Leonurus | sibiricus | Chito 02 |
| 1387 | 1 | Lamiaceae | Leonurus | sibiricus | MeJa 03 |
| 1395 | 1 | Ephedraceae | Ephedra | nevadensis | Acetate 03 |
| 1397 | 3 | Convolvulaceae | Ipomoea | purpurea | control 01 |
| 1399 | 3 | Convolvulaceae | Ipomoea | purpurea | Acetate 03 |
| 1401 | 2 | Convolvulaceae | Ipomoea | purpurea | AgN03 02 |
| 1403 | 2 | Convolvulaceae | Ipomoea | purpurea | Chito 02 |
| 1405 | 3 | Convolvulaceae | Ipomoea | purpurea | MeJa 03 |
| 1407 | 3 | Amaranthaceae | Cyathula | officinalis | Acetate 03 |
| 1409 | 3 | Asteraceae | Xanthium | sibiricum | Acetate 03 |
| 1413 | 3 | Fabaceae | Tephrosia | grandiflora | Acetate 03 |

TABLE 5
Sniffing Test on Cuticular Washings

| Sample # | Rating | Family | Genus | Species |
|---|---|---|---|---|
| wx 201 | 1 | Rutaceae | Murrays | exotica |
| wx 202 | 2 | Araliaceae | Trevesia | sungaica |
| wx 203 | 1 | Verbenaceae | Clerodendrum | speciosissimum |
| wx 204 | 0 | Euphorbiaceae | Acalypha | hispida |
| wx 205 | 2 | Gnetaceae | Gnetum | gnemon |
| wx 206 | 1 | Rubiaceae | Psychotria | nigropunctata |
| wx 207 | 1 | Rubiaceae | Psychotria | metbacteriodom |
| | | | | asica |
| wx 208 | 2 | Euphorbiaceae | Codiaeum | variegatum |
| wx 209 | 2 | Euphorbiaceae | Phyllanthus | grandifolius |
| wx 210 | 3 | Sterculiaceae | Pterigota | alata |
| wx 211 | 1 | Bombacaceae | Pachira | affinis |
| wx 212 | 1 | Sterculiaceae | Sterculia | elata |
| wx 213 | 0 | Araceae | Philodendron | speciosum |
| wx 214 | 2 | Fabaceae | Pithecellobium | unguis-cati |
| wx 215 | 1 | Acanthaceae | Sanchezia | nobilis |
| wx 216 | 1 | Araliaceae | Oreopanax | capitata |
| wx 217 | 0 | Moraceae | Ficus | triangularis |
| wx 218 | 2 | Bignoniaceae | Kigelia | pinnata |
| wx 219 | 1 | Poperaceae | Piper | cubeba |
| wx 220 | 3 | Lauraceae | Laurus | nobilis |
| wx 221 | 2 | Fabaceae | Erthrinia | crista-galli |
| wx 222 | 1 | Myrataceac | Metrosideros | excelsa |
| wx 223 | 3 | Oleaceae | Osmanthus | fragrans |
| wx 224 | 1 | Cupressaceae | Tetraclinis | articulata |
| wx 225 | 0 | Cupressaceae | Cupresus | sempervirens |
| wx 226 | 0 | Acanthaceac | Jacobinia | sp. |
| wx 227 | 0 | Asteraceae | Senecio | platyphylloides |
| wx 228 | 1 | Arecaceae | Livistona | chinensis |
| wx 229 | 3 | Myrataceac | Eucalyptus | rudis |
| wx 230 | 1 | Podocarpaceae | Podocarpus | spinulosus |
| wx 231 | 2 | Rosaceae | Eriobotrya | japonica |
| wx 232 | 2 | Ginkgoaceae | Gingko | biloba |
| wx 233 | 2 | Ericeae | Rhododendron | sp. |
| wx 234 | 2 | Cupressaceae | Thuja | occidentalis |
| wx 235 | 1 | Polygonaceae | Fagopyrum | suffruticosum |
| wx 236 | 1 | Rosaceae | Geum | macrophyllum |
| wx 237 | 2 | Magnoliaceae | Magnolia | kobus |
| wx 238 | 3 | Apocynaceae | Vinca | minor |
| wx 239 | 0 | Liliaceae | Convallaria | majalis |
| wx 240 | 2 | Betulaceae | Corvius | avellana |
| wx 241 | 2 | Berberidaceae | Berberis | sp. |
| wx 242 | 2 | Rosaceae | Rosa | multiflora |
| wx 243 | 1 | Betulaceae | Ostrya | carpinifolia |
| wx 244 | 1 | Betulaceae | Ostrya | connogea |
| wx 245 | 1 | Fagaceae | Quercus | rubra |
| wx 246 | 2 | Magnoliaceae | Liriodendron | tulipifera |
| wx 247 | 1 | Rosaceae | Sorbus | aucuparia |
| wx 248 | 3 | Betulaceae | Betula | nigra |
| wx 249 | 3 | Betulaceac | Betula | nigra |
| wx 250 | 2 | Fagaceae | Castanea | sativa |
| wx 251 | 0 | Saxofragaceae | Bergenia | crussifolia |
| wx 252 | 3 | Asteraceae | Artemisia | dracunculus |
| wx 253 | 3 | Rutaceae | Ruta | graveolens |
| wx 254 | 3 | Fragaceae | Quercus | nigra |
| wx 255 | 2 | Schisandraceae | Schisandra | chinensis |
| wx 256 | 3 | Betulaceac | Betula | alba |
| wx 257 | 2 | Caprifoliaceae | Sambucus | nigra |
| wx 258 | 3 | Actinicliaceae | Actiniclia | colonicta |
| wx 259 | 2 | Paeoniaceae | Paeonia | lactiflora |
| wx 260 | 1 | Paeoniaceae | Paeonia | suffructicisa |
| wx 261 | 0 | Fragaceae | Quercus | imbricaria |
| wx 262 | 1 | Indaceac | Iris | pallida |
| wx 263 | 2 | Portulaccaeae | Portulacca | oleracea |
| wx 264 | 2 | Polygonaceae | Polygonum | aviculare |
| wx 265 | 0 | Iriclaceae | Iris | pseudacorus |
| wx 266 | 1 | Liliaceae | Allium | nutans |
| wx 267 | 3 | Liliaceae | Allium | fistulosum |
| wx 268 | 1 | Liliaceae | Anthericum | ramosum |
| wx 269 | 1 | Liliaceae | Veratrum | nigrum |
| wx 270 | 1 | Polygonaceae | Polygonum | latifolia |
| wx 271 | 0 | Liliaceae | Hosta | lancifolia |
| wx 272 | 1 | Liliaceae | Hosta | zibalda |
| wx 273 | 2 | Asteraceae | Echinops | sphaerocephalus |
| wx 274 | 3 | Paeoniaceae | Paeonia | claurica |
| wx 275 | 1 | Asteraceae | Inula | helenium |
| wx 276 | 2 | Fabaceae | Crambe | pontica |
| wx 277 | 0 | Scrophuylari- | Digitalis | lutea |
| | | aceae | | |
| wx 278 | 3 | Fabraceae | Baptisia | australis |
| wx 279 | 2 | Aristolochi- | Aristolochia | clematitis |
| | | aceae | | |
| wx 280 | 3 | Lamiaceae | Hyssopus | zeraucharicus |
| wx 281 | 2 | Lamiaceae | Teucrium | hamedris |
| wx 282 | 2 | Crassulaceae | Sedum | album |
| wx 283 | 3 | Apiaceae | Heracleum | pubescens |
| wx 284 | 3 | Lamiaceae | Origanum | vulgare |
| wx 285 | 3 | Apiaceae | Cachrys | alpina. |
| wx 286 | 1 | Apiaceae | Laser | trilobum |
| wx 287 | 2 | Dryopterid- | Matteuccia | struthiopteris |
| | | aceae | | |
| wx 288 | 3 | Crassulaceae | Sedum | telephium |
| wx 289 | 2 | Papaveraceae | Bocconia | cordata |
| wx 290 | 1 | Lamiaceae | Ajuga | reptans |
| wx 291 | 1 | Ranunculaceae | Thalictrum | minus |
| wx 292 | 2 | Raminculaceae | Anemone | japonica |
| wx 293 | 1 | Ranunculaceae | Clematis | recta |
| wx 294 | 2 | Ranunculaceae | Thalictrum | sp. |
| wx 295 | 1 | Rosaceae | Alchemilla | sp. |
| wx 296 | 1 | Rosaceae | Potentilla | alba |
| wx 297 | 1 | Rosaeae | Poterium | sangiusorba |
| wx 298 | 3 | Menisperm- | Menispermum | dauricum |
| | | aceae | | |
| wx 299 | 3 | Nyctaginaceae | Oxybaphus | nyctagineus |
| wx 300 | 2 | Brassicaceae | Armoracia | rusticana |

The results presented hereinabove, demonstrate that plant exudates and components of cuticular coatings constitute a novel and important source of new biologically active compounds having antimicrobial, antifungal, insecticidal, sporicidal, cytotoxic activities and herbicidal properties that could be used for treatments of various diseases or conditions. In addition, compounds present in cuticular washings and root exudates can be used as fragrances, flavors, and flavor enhancers.

### Example 8

### Anti-microbial Activity Elicited by Acetic Acid

Anti-microbial activity was assessed using a whole-cell growth inhibition bioassay. More particularly, anti-fungal activity was assessed using Saccharomyces cerevisiae and Aspergillus niger; anti-bacterial activity was measured using Staphylococcus aureus (gram-positive) and Escherichia coli and Pseudomonas aeruginosa (gram-negative). To ensure that positive results reflected true anti-microbial activity, these test cells were subjected to the inhibition assay in the presence of acetic acid and in the absence of the elicitor (i.e., control).

Plants were germinated and grown, extracts were prepared, and bioassays were conducted as described above (see Examples 1, 2 and 6 in particular). Example 6 and Table 2 also indicate the wide variety of plants whose extracts were subjected to testing. In brief, plants were grown as described in Example 1, above. Following growth, plants were removed from the nutrient solution and placed in either distilled water (control) or in 0.1% (v/v) acetic acid, typically for 24 hours (see Example 1). Extracts of plant roots were generally prepared according to the protocol described in Example 4. In particular, plant roots (approximately 1-5 g fresh weight) were harvested and freeze-dried, followed by an extraction with 20 ml of 80% (v/v) methanol per gram of lyophilized roots at room temperature for 48 hours. The methanol-root compositions were then centrifuged and the supernatant was decanted. The extract was formed by drying the supernatant by evaporation. Typically, 100-120 μg of extract were used in each bioassay.

Microbial organisms used in the bioassays were grown as described (see Example 1). To conduct the bioassays, 24-well microtiter plates were used with growing microbes exposed to an extract (resulting from elicitation with acetic acid or a control extract resulting from exposure of the plant to water) or to a known inhibitor of microbe growth (kanamycin for bacteria; ketoconazole for fungi). In a fixed number of wells per plate, microbes (either bacteria, or fungi) were grown without presence of an extract and known antibiotic. The growth in these wells was used for comparison in assessing the growth inhibition potential of the plant extracts tested.

TABLE 6
Anti-microbial activity of extracts elicited with acetic acid

| Family | Genus | species | treatment | Staph | Ecoli | Sacchcer | Asping | Pseudom |
|---|---|---|---|---|---|---|---|---|
| Agavaceae | Agave | chrysantha | control 01 | | | 4, 4, 5 | | |
| Agavaceae | Agave | chrysantha | Acetate 03 | | | 4, 4, 5 | | |
| Agavaceae | Agave | neomexicana | control 01 | | | 4, 4, 5 | | |
| Agavaceae | Agave | neomexicana | Acetate 03 | | | 4, 4, 5 | | |
| Agavaceae | Yucca | baccata | control 01 | | | 5, 0 | | |
| Agavaceae | Yucca | baccata | Acetate 03 | | | 5, 0 | 3, 2 | |
| Agavaceae | Yucca | whipplei | control 01 | | | | | |
| Agavaceae | Yucca | whipplei | Acetate 03 | | | 5 | | |
| Aizoaceae | Aloinopsis | spathulata | control 01 | 2, 0, 1 | 1, 0, 1 | | | |
| Aizoaceae | Aloinopsis | spathulata | Acetate 03 | 2, 0, 1 | 1, 0, 1 | | | |
| Aizoaceae | Hypertelis | salsoloides | control 01 | | | | | |
| Aizoaceae | Hypertelis | salsoloides | Acetate 03 | 3 | | | | |
| Alliaceae | Allium | ampeloprasum | control 01 | | | 5, 5, 5 | | |
| Alliaceae | Allium | ampeloprasum | Acetate 03 | | | 5, 5, 5 | | |
| Amaranthaceae | Amaranthus | hypochondriacus | control 01 | 2 | | | | |
| Amaranthaceae | Amaranthus | hypochondriacus | Acetate 03 | 2 | | | | |
| Amaranthaceae | Amaranthus | powelli | control 01 | | | | | |
| Amaranthaceae | Amaranthus | powelli | Acetate 03 | | | 3 | | |
| Amaranthaceae | Amaranthus | retroflexus | control 01 | | | | | |
| Amaranthaceae | Amaranthus | retroflexus | Acetate 03 | 2 | | 2, 0, 0 | | |
| Amaryllidaceae | Agapanthus | africanus | control 01 | | | 5 | | |
| Amaryllidaceae | Agapanthus | africanus | Acetate 03 | | | 5 | 5 | |
| Anacardiaceae | Cotinus | coggygria | control 01 | 2 | 3 | 3 | | |
| Anacardiaceae | Cotinus | coggygria | Acetate 03 | 2 | | | | |
| Anacardiaceae | Malosma | laurina | control 01 | | | | | |
| Anacardiaceae | Malosma | laurina | Acetate 03 | | 2 | | | |
| Anacardiaceae | Rhus | integrifolia | control 01 | 3 | | | | |
| Anacardiaceae | Rhus | integrifolia | Acetate 03 | 3 | | | | |
| Anacardiaceae | Rhus | typhina | control 01 | 3 | | | | |
| Anacardiaceae | Rhus | typhina | Acetate 03 | 3 | | | | |
| Anacardiaceae | Schinus | terebinthifolius | control 01 | | | | | |
| Anacardiaceae | Schinus | terebinthifolius | Acetate 03 | 3 | | | | |
| Apiaceae | Actinotus | helianthi | control 01 | 4, 4 | 2, 0 | 2, 2 | 2, 2 | 1, 0 |
| Apiaceae | Actinotus | helianthi | Acetate 03 | 3, 0 | 1, 0 | | | 1, 0 |
| Apiaceae | Ammi | visnaga | control 01 | 4 | | | | |
| Apiaceae | Ammi | visnaga | Acetate 03 | 3 | | | | |
| Apiaceae | Angelica | archangelica | control 01 | 2 | | | | |
| Apiaceae | Angelica | archangelica | Acetate 03 | 2 | | 3 | | |
| Apiaceae | Anthriscus | cerefolium | control 01 | | | | | |
| Apiaceae | Anthriscus | cerefolium | Acetate 03 | 2 | | | | |
| Apiaceae | Anthriscus | cerefolium | control 01 | 4 | | 3 | 3 | |
| Apiaceae | Anthriscus | cerefolium | Acetate 03 | 4 | 3 | 4 | | 2 |
| Apiaceae | Bunium | bulbocastanum | control 01 | 5, 5 | 1, 0 | 4, 4 | | 1, 0 |
| Apiaceae | Bunium | bulbocastanum | Acetate 03 | 4, 4 | | 3, 3 | | 1, 2 |
| Apiaceae | Bupleurum | aureum | control 01 | 3, 4 | 2, 0 | 2, 0 | | 2, 0 |
| Apiaceae | Bupleurum | aureum | Acetate 03 | 2, 3 | 2, 2 | 2, 0 | | 2, 0 |
| Apiaceae | Carum | roseburghianum | control 01 | | | | | |
| Apiaceae | Carum | roseburghianum | Acetate 03 | 3 | | 3 | | |
| Apiaceae | Crithmum | maritimum | control 01 | 3, 3c | | | | |
| Apiaceae | Crithmum | maritimum | Acetate 03 | 3, 4c | | | 4, 4 | |
| Apiaceae | Cryptotaenia | japonica | control 01 | 4, 1, 4 | | 3, 0, 4 | | |
| Apiaceae | Cryptotaenia | japonica | Acetate 03 | 3, 0, 2 | | | | |
| Apiaceae | Cuminum | cyminum | control 01 | 2, 0, 0 | | | | |
| Apiaceae | Cuminum | cyminum | Acetate 03 | 1, 0, 0 | | | | |
| Apiaceae | Dasispermum | suffruticosum | control 01 | 4, 4 | | | 2, 0 | 3, 3 |
| Apiaceae | Dasispermum | suffruticosum | Acetate 03 | 4, 4 | | | 2, 4 | 4, 4 |
| Apiaceae | Ferula | communis | control 01 | 3c, 3c | | | 3, 2 | |
| Apiaceae | Ferula | communis | Acetate 03 | 3c, 2 | | | 2, 2 | |
| Apiaceae | Ferula | communis | control 01 | | | | | |
| Apiaceae | Ferula | communis | Acetate 03 | | | | | |
| Apiaceae | Foeniculum | vulgare | control 01 | 4, 5 | | 2, 2 | 2, 2 | |
| Apiaceae | Foeniculum | vulgare | Acetate 03 | 3, 4 | | 2, 1 | | |
| Apiaceae | Libanotis | montana | control 01 | | | 5 | | |
| Apiaceae | Libanotis | montana | Acetate 03 | | | 3 | | |
| Apiaceae | Ligusticum | porteri | control 01 | | 1, 0, 0 | | | |
| Apiaceae | Ligusticum | porteri | Acetate 03 | 2, 0, 0 | 1, 0, 1 | 3, 0, 0 | | |
| Apiaceae | Petroselinum | crispum | control 01 | | | | | |
| Apiaceae | Petroselinum | crispum | Acetate 03 | 3 | 2 | 4 | | |
| Apiaceae | Petroselinum | crispum | control 01 | | | | | |
| Apiaceae | Petroselinum | crispum | Acetate 03 | | | | | |
| Apiaceae | Petroselinum | crispum | control 01 | | | | | |
| Apiaceae | Petroselinum | crispum | Acetate 03 | | | | | |
| Apiaceae | Pimpinella | saxifraga | control 01 | 3 | | | | |
| Apiaceae | Pimpinella | saxifraga | Acetate 03 | 4 | | | | |
| Apiaceae | Sium | sisarum | control 01 | 3, 4 | | | | |
| Apiaceae | Sium | sisarum | Acetate 03 | 5, 4 | | | 2, 0 | 3, 3 |
| Apiaceae | Smyrnium | olusatrum | control 01 | | | | | 1, 1 |
| Apiaceae | Smyrnium | olusatrum | Acetate 03 | 1, 2 | | | | |
| Apiaceae | Steganotaenia | araliacea | control 01 | 5, 4 | | 1, 3 | 3, 3 | 2, 0 |
| Apiaceae | Steganotaenia | araliacea | Acetate 03 | 4, 4 | | 4, 4 | 4, 4 | 3, 0 |
| Apiaceae | Torilis | arvensis | Acetate 03 | 3c, 3 | | 2, 0 | 1, 2 | |
| Apiaceae | Trachymene | caerulea | control 01 | 1, 4 | 2, 0 | 1, 2 | 2, 2 | |
| Apiaceae | Trachymene | caerulea | Acetate 03 | 4, 4 | 2, 0 | 1, 2 | 2, 2 | |
| Apiaceae | Trachyspermum | ammi | control 01 | 4, 4 | | | | |
| Apiaceae | Trachyspermum | ammi | Acetate 03 | 5, 4 | | 2, 3 | 4, 3 | |
| Apiaceae | Zizia | aptera | control 01 | 5, 4 | 2, 0 | 3, 3 | 2, 4 | 2, 0 |
| Apiaceae | Zizia | aptera | Acetate 03 | 4, 4 | 2, 0 | 3, 2 | 2, 3 | 2, 0 |
| Apocynaceae | Catharanthus | roseus | control 01 | | | | | |
| Apocynaceae | Catharanthus | roseus | control 01 | | | | | |
| Apocynaceae | Catharanthus | roseus | control 01 | | | | | |
| Apocynaceae | Catharanthus | roseus | Acetate 01 | | | | | |
| Apocynaceae | Catharanthus | roseus | Acetate 01 | | | | | |
| Apocynaceae | Catharanthus | roseus | Acetate 01 | | | | | |
| Apocynaceae | Catharanthus | roseus | control 01 | 1, 0 | 3, 0 | | | |
| Apocynaceae | Catharanthus | roseus | Acetate 03 | | 3, 0 | | | |
| Araucariaceae | Araucaria | araucana | control 01 | 3, 2, 2 | | | | |
| Araucariaceae | Araucaria | araucana | Acetate 03 | 3, 2, 3 | | 2, 0, 0 | | |
| Arecaceae | Phoenix | dactylifera | control 01 | | | 5 | | |
| Arecaceae | Phoenix | dactylifera | Acetate 03 | | | 5 | | |
| Arecaceae | Phoenix | dactylifera | control 01 | | | 3 | 2 | |
| Arecaceae | Phoenix | dactylifera | Acetate 03 | | | 4 | 2 | |
| Arecaceae | Trachycarpus | excelsus | control 01 | | | 5 | | |
| Arecaceae | Trachycarpus | excelsus | Acetate 03 | | | 5 | | |
| Asphodelaceae | Kniphofia | baurii | control 01 | | | | | |
| Asphodelaceae | Kniphofia | baurii | Acetate 03 | 3 | | | | |
| Asteraceae | Achillea | filipendulina | control 01 | | | | | |
| Asteraceae | Achillea | filipendulina | Acetate 03 | 3 | | | | |
| Asteraceae | Achillea | millefolium | control 01 | | | | | |
| Asteraceae | Achillea | millefolium | control 01 | | | | | |
| Asteraceae | Achillea | millefolium | control 01 | | | | | |
| Asteraceae | Achillea | millefolium | Acetate 02 | | | | | |
| Asteraceae | Achillea | millefolium | Acetate 02 | | | | | |
| Asteraceae | Achillea | millefolium | Acetate 02 | | | | | |
| Asteraceae | Achillea | millefolium | control 01 | | | | | |
| Asteraceae | Achillea | millefolium | Acetate 03 | 4 | | | | |
| Asteraceae | Achillea | millefolium | control 01 | | | | | |
| Asteraceae | Achillea | millefolium | Acetate 03 | | | | | |
| Asteraceae | Agoseris | grandiflora | control 01 | | | | | |
| Asteraceae | Agoseris | grandiflora | Acetate 03 | 2, 0 | 2, 2 | | 2, 2 | |
| Asteraceae | Amellus | asteroides | control 01 | 2, 0 | | | | |
| Asteraceae | Amellus | asteroides | Acetate 03 | 1, 0 | 1, 0 | | | |
| Asteraceae | Anacyclus | pyrethrum | control 01 | | | 1, 1 | 3, 2 | |
| Asteraceae | Anacyclus | pyrethrum | Acetate 03 | 4, 3 | 2, 2 | 2, 2 | 3, 3 | 2, 2 |
| Asteraceae | Anthemis | nobilis | control 01 | | | | | |
| Asteraceae | Anthemis | nobilis | Acetate 03 | | | | | 2 |
| Asteraceae | Arctanthemum | arcticum | control 01 | 2, 1 | | | | |
| Asteraceae | Arctanthemum | arcticum | Acetate 03 | 2, 3 | | | 3, 0 | |
| Asteraceae | Arctotheca | calendula | control 01 | | | | | |
| Asteraceae | Arctotheca | calendula | Acetate 03 | 2, 0 | | | | |
| Asteraceae | Argyranthemum | frutescens | control 01 | 1, 1 | 1, 0 | | | |
| Asteraceae | Argyranthemum | frutescens | Acetate 03 | 2, 0 | | | | |
| Asteraceae | Artemisia | absinthium | control 01 | | | | | |
| Asteraceae | Artemisia | absinthium | Acetate 03 | 3 | | | | |
| Asteraceae | Artemisia | douglasiana | control 01 | | | | | |
| Asteraceae | Artemisia | douglasiana | Acetate 03 | 3 | | | | |
| Asteraceae | Artemisia | dracunculus | control 01 | 2 | | | | |
| Asteraceae | Artemisia | dracunculus | Acetate 03 | 4 | | | | |
| Asteraceae | Artemisia | suksdorfii | control 01 | | | | | |
| Asteraceae | Artemisia | suksdorfii | Acetate 03 | 4 | | | | |
| Asteraceae | Artemisia | suksdorfii | Acetate 03 | | | | | |
| Asteraceae | Athanasia | crithmifolia | control 01 | 2, 0 | | | | |
| Asteraceae | Athanasia | crithmifolia | Acetate 03 | 1, 3 | 1, 1 | | 3, 3 | |
| Asteraceae | Atractylodes | macrocephala | control 01 | | | | | |
| Asteraceae | Atractylodes | macrocephala | Acetate 03 | 3 | | | | |
| Asteraceae | Baccharis | emoryi | control 01 | 3c, 3c | | | 4, 2 | |
| Asteraceae | Baccharis | emoryi | Acetate 03 | 3c, 3c | 2, 2 | 2, 3 | 3, 2 | |
| Asteraceae | Berkheya | purpurea | control 01 | | | | | |
| Asteraceae | Berkheya | purpurea | Acetate 03 | | | | | 2, 3 |
| Asteraceae | Boltonia | decurrens | control 01 | | | | | |
| Asteraceae | Boltonia | decurrens | Acetate 03 | 2, 2 | | 1, 0 | | |
| Asteraceae | Bracteantha | macrantha | control 01 | 1, 1 | | 2, 0 | | 1, 0 |
| Asteraceae | Bracteantha | macrantha | Acetate 03 | 3, 3 | | 2, 2 | | 1, 0 |
| Asteraceae | Buphthalmum | salicifolium | control 01 | 3, 2 | 2, 2 | 1, 0 | | |
| Asteraceae | Buphthalmum | salicifolium | Acetate 03 | 4, 4 | 2, 2 | | | |
| Asteraceae | Callistephus | chinensis | control 01 | | 2, 1 | 4, 4 | | |
| Asteraceae | Callistephus | chinensis | Acetate 03 | 1, 2 | | 3, 4 | | |
| Asteraceae | Carduncellus | mutissimus | control 01 | 2, 1 | 1, 1 | | | 2, 0 |
| Asteraceae | Carduncellus | mutissimus | Acetate 03 | 2, 2 | | | 1, 0 | 1, 1 |
| Asteraceae | Carthamus | tinctorius | control 01 | | | | | |
| Asteraceae | Carthamus | tinctorius | Acetate 03 | 5 | 3 | | | |
| Asteraceae | Cassinia | aculeata | control 01 | 2, 0 | 1, 0 | | | 1, 1 |
| Asteraceae | Cassinia | aculeata | Acetate 03 | 3, 3 | 1, 1 | | 2, 3 | 1, 1 |
| Asteraceae | Centaurea | maculosa | control 01 | | | | | |
| Asteraceae | Centaurea | maculosa | Acetate 03 | 2, 1, 2 | | | | |
| Asteraceae | Chamaemelum | nobile | control 01 | | 3, 0 | | | |
| Asteraceae | Chamaemelum | nobile | Acetate 03 | | 2, 3 | | | |
| Asteraceae | Chrysanthemum | coronarium | control 01 | | | | | |
| Asteraceae | Chrysanthemum | coronarium | Acetate 03 | | | 2 | | |
| Asteraceae | Chrysanthemum | coronarium | control 01 | | | | | |
| Asteraceae | Chrysanthemum | coronarium | Acetate 03 | | | | | |
| Asteraceae | Chrysanthemum | leucanthemum | control 01 | | | | | |
| Asteraceae | Chrysanthemum | leucanthemum | Acetate 03 | 4 | | | | |
| Asteraceae | Chrysanthemum | parthenium | control 01 | | | | | |
| Asteraceae | Chrysanthemum | parthenium | Acetate 03 | 2 | | 5 | | |
| Asteraceae | Chrysothamnus | nauseosus | control 01 | 1, 0 | | | 2, 3 | |
| Asteraceae | Chrysothamnus | nauseosus | Acetate 03 | 2, 2 | | | 2, 2 | |
| Asteraceae | Cicerbita | alpina | control 01 | | | | | |
| Asteraceae | Cicerbita | alpina | Acetate 03 | 1, 0 | | | | |
| Asteraceae | Cirsium | vulgare | control 01 | | 1, 0 | | 1, 0 | |
| Asteraceae | Cirsium | vulgare | Acetate 03 | 4, 3 | | | 1, 0 | 1, 0 |
| Asteraceae | Cnicus | benedictus | control 01 | | 2 | | | |
| Asteraceae | Cnicus | benedictus | Acetate 03 | | 1 | | | |
| Asteraceae | Cnicus | benedictus | control 01 | 2 | | | | |
| Asteraceae | Cnicus | benedictus | Acetate 03 | 3 | 1 | | | |
| Asteraceae | Coleostephus | myconis | control 01 | 2, 0 | 2, 0 | | 3, 0 | 2, 0 |
| Asteraceae | Coleostephus | myconis | Acetate 03 | 1, 2 | | | 3, 2 | |
| Asteraceae | Conoclinium | coelestinum | control 01 | 2, 1 | 2, 0 | 2, 0 | | 1, 0 |
| Asteraceae | Conoclinium | coelestinum | Acetate 03 | 2, 2 | 3, 2 | 1, 0 | | 1, 0 |
| Asteraceae | Coreopsis | tinctoria | control 01 | 3 | | | | |
| Asteraceae | Coreopsis | tinctoria | Acetate 03 | 3 | | | | |
| Asteraceae | Corethrogyne | californica | control 01 | | | | | |
| Asteraceae | Corethrogyne | californica | Acetate 03 | 3 | | | | |
| Asteraceae | Doronicum | orientale | control 01 | | 1, 0 | | | 1, 0 |
| Asteraceae | Doronicum | orientale | Acetate 03 | 4, 3 | 3, 3 | | 2, 1 | 2, 2 |
| Asteraceae | Elephantopus | scaber | control 01 | | | 2, 2 | | |
| Asteraceae | Elephantopus | scaber | Acetate 03 | | 2, 0 | | | 2, 0 |
| Asteraceae | Emilia | coccinea | control 01 | | | 1, 0 | 3, 0 | |
| Asteraceae | Emilia | coccinea | Acetate 03 | 2, 4c | | 1, 2 | | 2, 0 |
| Asteraceae | Encelia | californica | control 01 | 3, 2 | 1, 2 | | 2, 0 | 1, 1 |
| Asteraceae | Encelia | californica | Acetate 03 | 4, 4 | | 2, 2 | 2, 1 | |
| Asteraceae | Eriophyllum | staechadifolium | control 01 | | | | | |
| Asteraceae | Eriophyllum | staechadifolium | Acetate 03 | | | 4 | | |
| Asteraceae | Eupatorium | cannabinum | control 01 | | | | | |
| Asteraceae | Eupatorium | cannabinum | Acetate 03 | | 2, 0, 1 | 1 | | |
| Asteraceae | Eupatorium | maculatum | control 01 | | | 3, 3, 2 | | |
| Asteraceae | Eupatorium | maculatum | Acetate 03 | 2, 2, 2 | | 4, 3, 2 | | |
| Asteraceae | Felicia | amelloides | control 01 | 2, 1 | 2, 2 | 1, 0 | | 2, 0 |
| Asteraceae | Felicia | amelloides | Acetate 03 | 2, 4 | 2, 4 | 4, 0 | | 4, 3 |
| Asteraceae | Felicia | echinata | control 01 | | | | | |
| Asteraceae | Felicia | echinata | Acetate 03 | 4, 0 | 2, 2 | | | |
| Asteraceae | Foveolina | tenella | control 01 | 1, 2 | 2, 3 | 2, 2 | | 2, 0 |
| Asteraceae | Foveolina | tenella | Acetate 03 | 2, 3 | 2, 0 | 3, 2 | 2, 2 | 2, 2 |
| Asteraceae | Gaillardia | aristata | control 01 | 4 | | | | |
| Asteraceae | Gaillardia | aristata | Acetate 03 | 2 | | | | |
| Asteraceae | Galactites | tomentosa | control 01 | 4, 1 | | 2, 2 | | |
| Asteraceae | Galactites | tomentosa | Acetate 03 | 4, 4 | | | 3, 3 | |
| Asteraceae | Geraea | viscida | control 01 | | | | | |
| Asteraceae | Geraea | viscida | Acetate 03 | 4, 3 | 2, 0 | | | 1, 0 |
| Asteraceae | Gnaphalium | californicum | control 01 | | | | 3, 2 | 1, 0 |
| Asteraceae | Gnaphalium | californicum | Acetate 03 | 4, 4 | | | 4, 0 | 2, 0 |
| Asteraceae | Heliopsis | helianthoides | control 01 | 1, 2 | | | | |
| Asteraceae | Heliopsis | helianthoides | Acetate 03 | 1, 3 | 2, 3 | | | 1, 0 |
| Asteraceae | Helipterum | argyropsis | control 01 | | | | | |
| Asteraceae | Helipterum | argyropsis | Acetate 03 | 1, 0 | | | | |
| Asteraceae | Hemizonia | congesta | control 01 | | 2, 1 | 1, 0 | | |
| Asteraceae | Hemizonia | congesta | Acetate 03 | 4, 3 | 2, 1 | 2, 2 | | 1, 1 |
| Asteraceae | Heterolepis | aliena | control 01 | | | | | |
| Asteraceae | Heterolepis | aliena | Acetate 03 | | | | 2, 2 | |
| Asteraceae | Heterolepis | aliena | Acetate 03 | 3, 2 | | | 2, 2 | |
| Asteraceae | Hulsea | heterochroma | control 01 | | | | 3, 0 | 2, 0 |
| Asteraceae | Hulsea | heterochroma | Acetate 03 | 3, 3c | 3, 0 | 4, 2 | 2, 3 | 2, 1 |
| Asteraceae | Hymenolepis | parviflora | control 01 | | | | | 2, 0 |
| Asteraceae | Hymenolepis | parviflora | Acetate 03 | 2, 2 | 2, 2 | | | 1, 0 |
| Asteraceae | Inula | ensifolia | control 01 | | | | | |
| Asteraceae | Inula | ensifolia | Acetate 03 | | 1, 0, 1 | | | |
| Asteraceae | Jurinea | mollis | control 01 | 2, 0 | | | | |
| Asteraceae | Jurinea | mollis | Acetate 03 | 4, 4 | | | | |
| Asteraceae | Lasiospermum | bipinnatum | control 01 | | | | 2, 2 | |
| Asteraceae | Lasiospermum | bipinnatum | Acetate 03 | 1, 3 | | | 2, 3 | |
| Asteraceae | Lasthenia | glabrata | control 01 | | | | | 2 |
| Asteraceae | Lasthenia | glabrata | Acetate 03 | | | | | 2 |
| Asteraceae | Leontodon | autumnalis | control 01 | | 2, 2 | 3, 2 | | |
| Asteraceae | Leontodon | autumnalis | Acetate 03 | | | 2, 0 | 3, 4 | |
| Asteraceae | Leucanthemopsis | alpina | control 01 | 2, 2 | 2, 0 | | | 1, 0 |
| Asteraceae | Leucanthemopsis | alpina | Acetate 03 | 2, 3 | 1, 1 | 1, 0 | 1, 2 | |
| Asteraceae | Leucheria | cerberoana | control 01 | | | | 4, 0 | |
| Asteraceae | Leucheria | cerberoana | Acetate 03 | 4, 4 | 1, 3 | 2, 2 | 2, 2 | |
| Asteraceae | Liatris | spicata | control 01 | | | | | |
| Asteraceae | Liatris | spicata | Acetate 03 | | 3, 4, 0 | | | |
| Asteraceae | Madia | elegans | control 01 | 1, 2 | | | | |
| Asteraceae | Madia | elegans | Acetate 03 | 4, 4 | 1, 1 | | 2, 0 | |
| Asteraceae | Malacothrix | californica | control 01 | 2, 0 | | | | |
| Asteraceae | Malacothrix | californica | Acetate 03 | 2, 4 | 2, 1 | 1, 1 | | 1, 1 |
| Asteraceae | Matricaria | matricarioides | control 01 | | | 2, 2 | | 1, 2 |
| Asteraceae | Matricaria | matricarioides | Acetate 03 | 1, 0 | 1, 2 | | | |
| Asteraceae | Neurolaena | lobata | control 01 | | | | | |
| Asteraceae | Neurolaena | lobata | Acetate 03 | 4, 5 | | 2, 4 | | |
| Asteraceae | Oldenburgia | grandis | control 01 | 2, 0 | | 3, 1 | | 2, 1 |
| Asteraceae | Oldenburgia | grandis | Acetate 03 | 1, 2 | 2, 2 | | 2, 0 | 2, 1 |
| Asteraceae | Oncosiphon | grandiflorum | control 01 | 3, 0 | | | | |
| Asteraceae | Oncosiphon | grandiflorum | Acetate 03 | 3, 0 | 1, 2 | | | |
| Asteraceae | Onopordum | acanthium | control 01 | | 1, 0 | | | |
| Asteraceae | Onopordum | acanthium | Acetate 03 | 1, 2 | 2, 2 | 2, 2 | | |
| Asteraceae | Phagnalon | saxatile | control 01 | | | | | |
| Asteraceae | Phagnalon | saxatile | Acetate 03 | 2, 2 | 2, 0 | 1, 0 | | |
| Asteraceae | Phymaspermum | acerosum | control 01 | 1, 1 | 2, 2 | | 1, 1 | |
| Asteraceae | Phymaspermum | acerosum | Acetate 03 | 3c, 2 | 1, 2 | | | |
| Asteraceae | Picris | echioides | control 01 | | 1, 0 | 1, 0 | 1, 0 | |
| Asteraceae | Picris | echioides | Acetate 03 | | 3, 1 | | 1, 0 | |
| Asteraceae | Polymnia | uvedalia | control 01 | | 3, 0 | 1, 0 | | 2, 2 |
| Asteraceae | Polymnia | uvedalia | Acetate 03 | 4, 4 | 2, 0 | 2, 3 | | 2, 2 |
| Asteraceae | Porophyllum | ruderale | control 01 | | | | | |
| Asteraceae | Porophyllum | ruderale | Acetate 03 | 5 | | | | |
| Asteraceae | Ptilostemon | afer | control 01 | 4, 4 | | 2, 2 | 3, 0 | 2, 0 |
| Asteraceae | Ptilostemon | afer | Acetate 03 | 4, 4 | | 1, 2 | 4, 0 | 1, 0 |
| Asteraceae | Pulicaria | dysenterica | control 01 | 2, 0 | 3, 2 | | | |
| Asteraceae | Pulicaria | dysenterica | Acetate 03 | 3, 3 | | 2, 0 | | |
| Asteraceae | Pyrethrum | corymbosum | control 01 | | | | | |
| Asteraceae | Pyrethrum | corymbosum | control 01 | | | | | |
| Asteraceae | Pyrethrum | corymbosum | Acetate 03 | 2, 2 | 2, 1 | 3, 2 | | 3, 2 |
| Asteraceae | Saussurea | heteromala | control 01 | 3, 0 | | | | 1, 0 |
| Asteraceae | Saussurea | heteromala | Acetate 03 | 3, 2 | | | | |
| Asteraceae | Scorzonera | hispanica | control 01 | 3, 1 | 2, 0 | | | |
| Asteraceae | Scorzonera | hispanica | Acetate 03 | 2, 1 | | | | 1, 1 |
| Asteraceae | Senecio | cineraria | control 01 | | | | | |
| Asteraceae | Senecio | cineraria | Acetate 03 | 3 | | | | |
| Asteraceae | Silybum | marianum | control 01 | | | | | |
| Asteraceae | Silybum | marianum | Acetate 03 | 3, 3, 3 | | 0, 0, 3 | | |
| Asteraceae | Sinacalia | tangutica | control 01 | | 1, 0 | | | 1, 0 |
| Asteraceae | Sinacalia | tangutica | Acetate 03 | 4, 3 | | 2, 0 | 3, 0 | 3, 0 |
| Asteraceae | Solidago | virgaurea | control 01 | | | | | |
| Asteraceae | Solidago | virgaurea | Acetate 03 | 4 | | 4 | | |
| Asteraceae | Solidago | virgaurea | control 01 | | | | | |
| Asteraceae | Sonchus | oleraceus | control 01 | 2, 2 | 3, 1 | 3, 2 | | 2, 0 |
| Asteraceae | Sonchus | oleraceus | Acetate 03 | 4, 4 | 2, 2 | 2, 2 | | 2, 1 |
| Asteraceae | Stephanomeria | virgata | control 01 | 2, 2 | 2, 0 | 2, 2 | 2, 2 | 2, 0 |
| Asteraceae | Stephanomeria | virgata | Acetate 03 | 3, 3c | 2, 0 | 2, 2 | 3, 2 | 2, 0 |
| Asteraceae | Tanacetum | camphoratum | control 01 | | 1, 2 | | | 2, 1 |
| Asteraceae | Tanacetum | camphoratum | Acetate 03 | 1, 0 | | | | 1, 2 |
| Asteraceae | Tripleurospermum | inodorum | control 01 | | 1, 0 | | | |
| Asteraceae | Tripleurospermum | inodorum | Acetate 03 | | 3, 2 | | | |
| Asteraceae | Venegasia | carpesioides | control 01 | | | | 4, 0 | 2, 0 |
| Asteraceae | Venegasia | carpesioides | Acetate 03 | 4, 3c | 2, 0 | | 3, 3 | |
| Asteraceae | Viguiera | laciniata | control 01 | 2, 1 | 2, 2 | | | 2, 3 |
| Asteraceae | Viguiera | laciniata | Acetate 03 | 3c, 3c | 2, 1 | 3, 4 | 2, 2 | 2, 2 |
| Asteraceae | Wedelia | biflora | control 01 | 1, 0 | | | | |
| Asteraceae | Wedelia | biflora | Acetate 03 | 2, 3 | | | 2, 0 | |
| Asteraceae | Xanthisma | texanum | control 01 | 3, 2 | | | | |
| Asteraceae | Xanthisma | texanum | Acetate 03 | 4, 3 | | | | 1, 0 |
| Asteraceae | Xanthium | strumarium | control 01 | | | 1, 0, 0 | | |
| Asteraceae | Xanthium | strumarium | Acetate 03 | | | | | |
| Asteraceae | Xeranthemum | annuum | control 01 | 2, 3c | | 2, 2 | | |
| Asteraceae | Xeranthemum | annuum | Acetate 03 | 3, 3 | | | 3, 0 | |
| Berberidaceae | Berberis | thunbergii | control 01 | | | | | |
| Berberidaceae | Berberis | thunbergii | Acetate 03 | 2, 4, 4 | | 0, 0, 2 | | |
| Berberidaceae | Berberis | thunbergii | control 01 | | | | | |
| Berberidaceae | Berberis | thunbergii | Acetate 03 | | | | | |
| Berberidaceae | Nandina | domestica | control 01 | | | | | |
| Berberidaceae | Nandina | domestica | control 01 | | | | | |
| Berberidaceae | Nandina | domestica | Acetate 03 | 3 | | | | |
| Berberidaceae | Podophyllum | emodii | control 01 | | | | | |
| Berberidaceae | Podophyllum | emodii | Acetate 03 | 2, 0, 3 | | | | |
| Berberidaceae | Podophyllum | hexandrum | control 01 | 3, 2, 3 | | | | |
| Berberidaceae | Podophyllum | hexandrum | Acetate 03 | 3, 2, 3 | | | | |
| Betulaceae | Carpinus | orientalis | control 01 | | | 1, 0 | | 2, 1 |
| Betulaceae | Carpinus | orientalis | Acetate 03 | | | | | |
| Bignoniaceae | Chilopsis | linearis | control 01 | 1, 0, 1 | | | | |
| Bignoniaceae | Chilopsis | linearis | Acetate 03 | | | | | |
| Bignoniaceae | Chilopsis | linearis | control 01 | | | | | |
| Bignoniaceae | Chilopsis | linearis | Acetate 03 | | | | | |
| Boraginaceae | Anchusa | officinalis | control 01 | | | | | |
| Boraginaceae | Anchusa | officinalis | Acetate 03 | 1, 0, 3 | | | | |
| Boraginaceae | Myosotis | sylvatica | control 01 | | | | | |
| Boraginaceae | Myosotis | sylvatica | Acetate 03 | 5 | | | | |
| Brassicaceae | Brassica | hirta | control 01 | 2, 0, 0 | 2, 0, 1 | | | |
| Brassicaceae | Brassica | hirta | Acetate 03 | 1 | 2, 0, 2 | | | |
| Brassicaceae | Brassica | oleracea | control 01 | | | | | |
| Brassicaceae | Brassica | oleracea | control 01 | | | | | |
| Brassicaceae | Brassica | oleracea | control 01 | | | | | |
| Brassicaceae | Brassica | oleracea | Acetate 01 | | | | | |
| Brassicaceae | Brassica | oleracea | Acetate 01 | | | | | |
| Brassicaceae | Brassica | oleracea | Acetate 01 | | | | | |
| Brassicaceae | Brassica | oleracea | control 01 | | | | | |
| Brassicaceae | Brassica | oleracea | control 01 | | | | | |
| Brassicaceae | Brassica | oleracea | control 01 | | | | | |
| Brassicaceae | Brassica | oleracea | Acetate 01 | | | | | |
| Brassicaceae | Brassica | oleracea | Acetate 01 | | | | | |
| Brassicaceae | Brassica | oleracea | Acetate 01 | | | | | |
| Brassicaceae | Brassica | oleracea | control 01 | 1 | | | | |
| Brassicaceae | Brassica | oleracea | Acetate 03 | | | | | |
| Brassicaceae | Brassica | rapa | control 01 | | | 2 | | |
| Brassicaceae | Brassica | rapa | Acetate 03 | | 3, 1, 0 | | | |
| Brassicaceae | Brassica | rapa | control 01 | | | | | |
| Brassicaceae | Brassica | rapa | Acetate 03 | | | | | |
| Brassicaceae | Brassica | rapa | control 01 | | | | | |
| Brassicaceae | Brassica | rapa | Acetate 03 | | | | | |
| Brassicaceae | Iberis | coronaria | control 01 | 3 | 3 | | | |
| Brassicaceae | Iberis | coronaria | Acetate 03 | 2 | 1 | | | |
| Brassicaceae | Iberis | umbellata | control 01 | | | | | |
| Brassicaceae | Iberis | umbellata | Acetate 03 | | 2 | 3 | | |
| Brassicaceae | Isatis | tinctoria | control 01 | 2 | | | | |
| Brassicaceae | Isatis | tinctoria | Acetate 03 | 2 | | | | |
| Brassicaceae | Isatis | tinctoria | control 01 | | | | | |
| Brassicaceae | Isatis | tinctoria | Acetate 03 | | | | | |
| Brassicaceae | Nasturtium | officinale | control 01 | 2 | | | | |
| Brassicaceae | Nasturtium | officinale | Acetate 03 | 2 | | | | |
| Buxaceae | Buxus | sinica | control 01 | 2, 0, 0 | 1, 0, 0 | | | |
| Campanulaceae | Adenophora | bulleyana | control 01 | | | | | |
| Campanulaceae | Adenophora | bulleyana | Acetate 03 | 1, 0, 1 | | | | |
| Campanulaceae | Adenophora | bulleyana | control 01 | | | | | |
| Campanulaceae | Adenophora | bulleyana | Acetate 03 | 3 | | | | |
| Campanulaceae | Campanula | persicifolia | control 01 | | 1, 0, 0 | | | |
| Campanulaceae | Campanula | persicifolia | Acetate 03 | | | | | |
| Campanulaceae | Codonopsis | pilosula | control 01 | | | | | |
| Campanulaceae | Codonopsis | pilosula | Acetate 03 | 3, 0, 1 | | 2, 0, 3 | | |
| Campanulaceae | Lobelia | siphilitica | control 01 | | 1 | | 1 | |
| Campanulaceae | Lobelia | siphilitica | Acetate 03 | | | | | |
| Campanulaceae | Platycodon | grandiflorum | control 01 | | 1, 0, 0 | | | |
| Campanulaceae | Platycodon | grandiflorum | Acetate 03 | | 1, 0, 0 | | | |
| Caprifoliaceae | Sambucus | caerulea | control 01 | | | | 3, 2 | 1, 2 |
| Caprifoliaceae | Sambucus | caerulea | Acetate 03 | 2, 2 | 2, 2 | | 3, 2 | 1, 1 |
| Caprifoliaceae | Sambucus | tigranii | control 01 | 2, 0, 0 | | | | |
| Caprifoliaceae | Sambucus | tigranii | Acetate 03 | | | | | |
| Caprifoliaceae | Symphoricarpus | albus | control 01 | 2 | | | | |
| Caprifoliaceae | Symphoricarpus | albus | Acetate 03 | 3 | | | | |
| Caryophyllaceae | Agrostemma | githago | control 01 | 3, 0 | 2, 0 | 2, 0 | | 1, 1 |
| Caryophyllaceae | Agrostemma | githago | Acetate 03 | 3, 2 | 2, 0 | | | 1, 2 |
| Caryophyllaceae | Gypsophila | paniculata | control 01 | | | | | |
| Caryophyllaceae | Gypsophila | paniculata | Acetate 03 | | 1, 0, 0 | | | |
| Caryophyllaceae | Herniaria | glabra | control 01 | | | | | |
| Caryophyllaceae | Herniaria | glabra | Acetate 03 | 2 | | | | |
| Caryophyllaceae | Lychnis | alba | control 01 | 1 | | | | |
| Caryophyllaceae | Lychnis | alba | Acetate 03 | 1 | | | | |
| Caryophyllaceae | Lychnis | chalcedonica | control 01 | | | | 3, 3 | |
| Caryophyllaceae | Lychnis | chalcedonica | Acetate 03 | | | | 3, 3 | |
| Caryophyllaceae | Scleranthus | biflorus | control 01 | | | 2, 0 | 2, 0 | |
| Caryophyllaceae | Scleranthus | biflorus | Acetate 03 | | | 2, 0 | 2, 0 | |
| Caryophyllaceae | Silene | alba | control 01 | 2, 1 | 2, 0 | 2, 2 | | 2, 2 |
| Caryophyllaceae | Silene | alba | Acetate 03 | 2, 3 | 2, 2 | | 3, 4 | 2, 2 |
| Caryophyllaceae | Silene | armeria | control 01 | 1, 0 | | 3, 0 | 2, 2 | 2, 2 |
| Caryophyllaceae | Silene | armeria | Acetate 03 | 4, 4 | 1, 1 | 3, 2 | 4, 4 | 2, 1 |
| Celastraceae | Euonymus | koopmannii | control 01 | 1, 1, 1 | | | | |
| Celastraceae | Euonymus | koopmannii | Acetate 03 | | | | | |
| Chenopodiaceae | Chenopodium | botrys | control 01 | | | | | |
| Chenopodiaceae | Chenopodium | botrys | Acetate 03 | 2 | | | | |
| Cistaceae | Fumana | procumbens | control 01 | | | | | |
| Cistaceae | Fumana | procumbens | Acetate 03 | | | | | |
| Cistaceae | Fumana | procumbens | control 01 | | | | | |
| Cistaceae | Fumana | procumbens | control 01 | | | | | 1, 0 |
| Cistaceae | Fumana | procumbens | Acetate 03 | | 1, 0 | | | 2, 0 |
| Clusiaceae | Hypericum | perforatum | control 01 | 3, 0, 2 | 1 | | | |
| Clusiaceae | Hypericum | perforatum | Acetate 03 | 3, 0, 1 | | | | |
| Colchicaceae | Gloriosa | superba | control 01 | | | | | |
| Colchicaceae | Gloriosa | superba | Acetate 03 | | | 1 | 3 | |
| Combretaceae | Terminalia | arjuna | control 01 | 4, 3 | 2, 2 | 3, 2 | | 2, 2 |
| Combretaceae | Terminalia | arjuna | Acetate 03 | 2, 3 | 2, 2 | 3, 2 | | 2, 2 |
| Commelinaceae | Commelina | coelestis | control 01 | 1, 0 | | | 2, 0 | 1, 1 |
| Commelinaceae | Commelina | coelestis | Acetate 03 | 2, 2 | 2, 1 | 2, 2 | 3, 2 | 1, 1 |
| Commelinaceae | Cyanotis | speciosa | control 01 | | | | | |
| Commelinaceae | Cyanotis | speciosa | Acetate 03 | | | 5 | | |
| Commelinaceae | Tinantia | erecta | control 01 | 1, 0 | 1, 0 | 2, 1 | 2, 0 | 2, 0 |
| Commelinaceae | Tinantia | erecta | Acetate 03 | 4, 4 | 1, 1 | 5, 3 | 2, 0 | 1, 1 |
| Convolvulaceae | Mina | lobata | control 01 | | | | | |
| Convolvulaceae | Mina | lobata | Acetate 03 | 4 | | 4 | | |
| Cucurbitaceae | Cucumis | sativus | control 01 | | | | | |
| Cucurbitaceae | Cucumis | sativus | control 01 | | | | | |
| Cucurbitaceae | Cucumis | sativus | control 01 | | | | | |
| Cucurbitaceae | Cucumis | sativus | Acetate 01 | | | | | |
| Cucurbitaceae | Cucumis | sativus | Acetate 01 | | | | | |
| Cucurbitaceae | Cucumis | sativus | Acetate 01 | | | | | |
| Cucurbitaceae | Cucumis | sativus | control 01 | | | | | |
| Cucurbitaceae | Cucumis | sativus | Acetate 03 | 3, 0, 2 | 2, 0, 0 | | | |
| Cucurbitaceae | Trichosanthes | anguina | control 01 | | | | 3, 3 | |
| Cucurbitaceae | Trichosanthes | anguina | Acetate 03 | | | | 3, 3 | |
| Cuperssaceae | Thuja | orientalis | Acetate 03 | 3, 3, 3 | | | | |
| Cuperssaceae | Thuja | orientalis | Acetate 03 | 3, 3, 3 | | | | |
| Ebenaceae | Diospyros | kaki | control 01 | | | | | |
| Ebenaceae | Diospyros | kaki | Acetate 03 | 3, 0, 0 | | | | |
| Elaeagnaceae | Elaeagnus | commutata | control 01 | | | | | |
| Elaeagnaceae | Elaeagnus | commutata | Acetate 03 | 1, 1, 0 | | | | |
| Ericaceae | Rhododendron | luteum | control 01 | | | | | |
| Ericaceae | Rhododendron | luteum | Acetate 03 | | 2 | | | |
| Euphorbiaceae | Pseudolachnostylis | maprouneifolia | control 01 | | | | | |
| Euphorbiaceae | Pseudolachnostylis | maprouneifolia | Acetate 03 | | | | | 2 |
| Fabaceae | Albizia | julibrissin | control 01 | | | 3 | | |
| Fabaceae | Albizia | julibrissin | Acetate 03 | | | 3 | | |
| Fabaceae | Albizia | julibrissin | control 01 | | | | | |
| Fabaceae | Albizia | julibrissin | Acetate 03 | 4 | | | | |
| Fabaceae | Albizia | julibrissin | control 01 | 1, 0 | | | | |
| Fabaceae | Albizia | julibrissin | Acetate 03 | 2, 2 | | | | |
| Fabaceae | Albizia | kolomikta | control 01 | 3 | | | | |
| Fabaceae | Albizia | kolomikta | Acetate 03 | 3 | | | | |
| Fabaceae | Amorpha | fruticosa | control 01 | 1, 0 | | 2, 1 | 2, 2 | |
| Fabaceae | Amorpha | fruticosa | Acetate 03 | 1, 2 | 2, 2 | 2, 0 | 2, 0 | 1, 1 |
| Fabaceae | Cassia | tora | control 01 | | | | | |
| Fabaceae | Cassia | tora | Acetate 03 | 3 | | | | |
| Fabaceae | Ceratonia | siliqua | control 01 | | | | | |
| Fabaceae | Ceratonia | siliqua | Acetate 03 | 3 | | | | |
| Fabaceae | Ceratonia | siliqua | control 01 | | | | | |
| Fabaceae | Ceratonia | siliqua | Acetate 03 | | | | | |
| Fabaceae | Cicer | arietinum | control 01 | | | | | |
| Fabaceae | Cicer | arietinum | Acetate 03 | 1, 0, 3 | | | | |
| Fabaceae | Clitoria | ternatea | control 01 | 2 | | | 5, 4 | |
| Fabaceae | Clitoria | ternatea | Acetate 03 | 2 | | | 4 | |
| Fabaceae | Cytissus | scoparius | control 01 | 3, 2, 2 | | | | |
| Fabaceae | Cytissus | scoparius | acetate 03 | 1, 2, 0 | | | | |
| Fabaceae | Dalea | candida | control 01 | 3, 3, 2 | | | | |
| Fabaceae | Dalea | candida | control up | 3, 3, 2 | | | | |
| Fabaceae | Dalea | candida | Acetate 03 | 3, 3, 2 | | | | |
| Fabaceae | Erythrina | corallodendron | control 01 | | | | | |
| Fabaceae | Erythrina | corallodendron | Acetate 03 | 2 | | | | |
| Fabaceae | Gleditsia | caspica | control 01 | | 3, 1 | | 1, 0 | |
| Fabaceae | Gleditsia | caspica | Acetate 03 | 2, 3 | 1, 1 | 2, 1 | | |
| Fabaceae | Gleditsia | triacanthos | control 01 | | 3, 0 | | 1, 0 | |
| Fabaceae | Gleditsia | triacanthos | Acetate 03 | 1, 2 | 2, 1 | | 2, 0 | |
| Fabaceae | Gleditsia | triacanthos | Acetate 03 | | 3, 2 | | | 2, 1 |
| Fabaceae | Glycyrrhiza | echinata (glabra?) | control 01 | | | | | |
| Fabaceae | Glycyrrhiza | echinata (glabra?) | Acetate 03 | 2 | | | | |
| Fabaceae | Gymnocladus | dioicus | control 01 | | | | | |
| Fabaceae | Gymnocladus | dioicus | Acetate 03 | | 2 | | | |
| Fabaceae | Laburnum | anagyroides | control 01 | 4 | | | | |
| Fabaceae | Laburnum | anagyroides | Acetate 03 | 4 | | | | |
| Fabaceae | Laburnum | anagyroides | control 01 | | | | | |
| Fabaceae | Laburnum | anagyroides | Acetate 03 | | | | | |
| Fabaceae | Leucaena | leucocephala | control 01 | 2, 2 | 3, 0 | 2, 0 | | |
| Fabaceae | Leucaena | leucocephala | Acetate 03 | 3, 2 | | | | |
| Fabaceae | Lupinus | bicolor | control 01 | 2 | | | | |
| Fabaceae | Lupinus | bicolor | Acetate 03 | 2 | | | | |
| Fabaceae | Mimosa | pudica | control 01 | 4 | | 2 | | |
| Fabaceae | Mimosa | pudica | Acetate 03 | 4 | | 2 | | |
| Fabaceae | Ononis | spinosa | control 01 | | | | | |
| Fabaceae | Ononis | spinosa | Acetate 03 | 2 | | | | |
| Fabaceae | Pachyrhizus | erosus | control 01 | | | | | |
| Fabaceae | Pachyrhizus | erosus | Acetate 03 | 2, 2, 3 | | | | |
| Fabaceae | Parkinsonia | aculeata | control 01 | | | | | |
| Fabaceae | Parkinsonia | aculeata | Acetate 03 | 3, 2 | | | | |
| Fabaceae | Parkinsonia | aculeata | Acetate 03 | | 2, 0 | | | |
| Fabaceae | Peltophorum | pterocarpum | control 01 | | | 2 | | |
| Fabaceae | Peltophorum | pterocarpum | Acetate 03 | | | | 4 | |
| Fabaceae | Petalostemon | candidum | control 01 | 5 | | | | |
| Fabaceae | Petalostemon | candidum | Acetate 03 | 5 | | | | |
| Fabaceae | Pithecellobium | dulce | control 01 | | 1, 2 | | | |
| Fabaceae | Pithecellobium | dulce | Acetate 03 | | 1, 1 | | 3, 0 | |
| Fabaceae | Prosopis | cineraria | control 01 | | | 3, 1 | | |
| Fabaceae | Prosopis | cineraria | Acetate 03 | | | 2, 1 | | |
| Fabaceae | Pueraria | lobata | control 01 | | | | | |
| Fabaceae | Pueraria | lobata | Acetate 03 | 2 | 2 | | | |
| Fabaceae | Schrankia | occidentalis | control 01 | | | | | |
| Fabaceae | Schrankia | occidentalis | Acetate 03 | 1, 3, 1 | | | | |
| Fabaceae | Sophora | japonica | control 01 | 3 | | | | |
| Fabaceae | Sophora | japonica | Acetate 03 | 3 | | | | |
| Fabaceae | Spartium | junceum | control 01 | | | | | |
| Fabaceae | Spartium | junceum | Acetate 03 | | | | | |
| Fabaceae | Spartium | junceum | control 01 | 3, 0 | | | | 2, 3 |
| Fabaceae | Spartium | junceum | Acetate 03 | 3, 0 | | | | |
| Fabaceae | Thermopsis | fabacea | control 01 | | | | | |
| Fabaceae | Thermopsis | fabacea | Acetate 03 | | | | | |
| Fabaceae | Thermopsis | fabacea | control 01 | | | | | |
| Fabaceae | Thermopsis | fabacea | Acetate 03 | 4 | | | 3 | |
| Fabaceae | Thermopsis | montana | control 01 | | | | | |
| Fabaceae | Thermopsis | montana | Acetate 03 | 3, 0, 0 | | | | |
| Fagaceae | Fagus | sylvatica | control 01 | 1, 0 | | | 2, 0 | 2, 0 |
| Fagaceae | Fagus | sylvatica | Acetate 03 | 1, 2 | | | | |
| Flacourtiaceae | Dovyalis | caffra | control 01 | 3 | | | 4, 3 | |
| Flacourtiaceae | Dovyalis | caffra | Acetate 03 | 3 | | | 4, 3 | |
| Gentianaceae | Centaurium | littorale | control 01 | | | | | |
| Gentianaceae | Centaurium | littorale | Acetate 03 | 1, 0, 0 | | | 1 | |
| Gentianaceae | Gentiana | macrophylla | control 01 | | | | 1 | |
| Gentianaceae | Gentiana | macrophylla | Acetate 03 | | | | 1 | |
| Gentianaceae | Gentiana | tibetica | control 01 | | | | | |
| Gentianaceae | Gentiana | tibetica | Acetate 03 | | 4, 0, 0 | 3, 0, 2 | | |
| Geraniaceae | Erodium | cicutarium | control 01 | | | | | |
| Geraniaceae | Erodium | cicutarium | Acetate 03 | 2, 0, 0 | 1, 0, 0 | | | |
| Geraniaceae | Geranium | macrorrhizum | control 01 | | | | | |
| Geraniaceae | Geranium | macrorrhizum | control 01 | 4, 2 | | | | |
| Geraniaceae | Geranium | macrorrhizum | Acetate 03 | 3, 0 | 3, 0 | 1, 0 | 2, 0 | |
| Hippocastanaceae | Aesculus | hippocastanum | control 01 | | | 5, 5, 5 | | |
| Hippocastanaceae | Aesculus | hippocastanum | Acetate 03 | | | 5, 5, 5 | | |
| Hippocastanaceae | Aesculus | woerlitzensis | control 01 | | | 5, 5, 5 | | |
| Hippocastanaceae | Aesculus | woerlitzensis | Acetate 03 | | | 5, 5, 5 | | |
| Hyacinthaceae | Chlorogalum | pomeridianum | control 01 | | | 5 | | |
| Hyacinthaceae | Chlorogalum | pomeridianum | Acetate 03 | | | 5 | | |
| Hyacinthaceae | Hosta | fortunea | control 01 | | | 5, 5, 5 | | |
| Hyacinthaceae | Hosta | fortunea | Acetate 03 | | | 5, 5, 5 | | |
| Hydrophyllaceae | Nemophila | maculata | control 01 | | | | | |
| Hydrophyllaceae | Nemophila | maculata | Acetate 03 | | 2 | | | |
| Hydrophyllaceae | Nemophila | maculata | control 01 | | | | | |
| Hydrophyllaceae | Nemophila | maculata | Acetate 03 | | | | | |
| Hydrophyllaceae | Phacelia | tanacetifolia | control 01 | | | | | |
| Hydrophyllaceae | Phacelia | tanacetifolia | Acetate 03 | 2 | 1, 0, 0 | | | |
| Iridaceae | Dierama | pulcherrimum | control 01 | | | | | |
| Iridaceae | Dierama | pulcherrimum | Acetate 03 | | | | 4, 3 | |
| Lamiaceae | Leonurus | cardiaca | control 01 | | | | | |
| Lamiaceae | Leonurus | cardiaca | Acetate 03 | 4 | | | | |
| Lamiaceae | Monarda | citriodora | control 01 | | | | | 2 |
| Lamiaceae | Monarda | citriodora | Acetate 03 | | | | | 2 |
| Lamiaceae | Monarda | citriodora | control 01 | 5 | | | | |
| Lamiaceae | Monarda | citriodora | Acetate 03 | 4 | | | | |
| Lamiaceae | Ocimum | sanctum | control 01 | | | | | |
| Lamiaceae | Ocimum | sanctum | Acetate 03 | | | | | |
| Lamiaceae | Ocimum | sanctum | control 01 | | | | 3, 0 | |
| Lamiaceae | Ocimum | sanctum | Acetate 03 | | | | 2, 0 | |
| Lamiaceae | Pogostemon | patchouli | control 01 | | | | | |
| Lamiaceae | Pogostemon | patchouli | Acetate 03 | 1 | | | | |
| Lamiaceae | Salvia | dumetorum | control 01 | | | | | |
| Lamiaceae | Salvia | dumetorum | Acetate 03 | 3, 4, 4 | | 2, 0, 3 | | |
| Lamiaceae | Salvia | mellifera | control 01 | | 1, 0, 0 | | | |
| Lamiaceae | Salvia | mellifera | Acetate 03 | | 1, 0, 0 | | | |
| Lamiaceae | Schizonepeta | tenuifolia | control 01 | | | | | |
| Lamiaceae | Schizonepeta | tenuifolia | Acetate 03 | 3 | | | | |
| Lamiaceae | Stachys | officinalis | control 01 | | | | | |
| Lamiaceae | Stachys | officinalis | Acetate 03 | 3 | | | | |
| Liliaceae | Cordyline | australis | control 01 | | 2, 3 | | 2, 0 | |
| Liliaceae | Cordyline | australis | Acetate 03 | | | 4, 5 | 2, 0 | |
| Liliaceae | Phormium | tenax | control 01 | 1, 2 | 2, 0 | | | |
| Liliaceae | Phormium | tenax | Acetate 03 | 2, 2 | 2, 0 | | | |
| Linaceae | Linum | grandiflorum | control 01 | | | | | |
| Linaceae | Linum | grandiflorum | Acetate 03 | | | | | |
| Linaceae | Linum | grandiflorum | control 01 | | | | | |
| Linaceae | Linum | grandiflorum | Acetate 03 | 2 | 2 | 2 | | |
| Linaceae | Linum | perenne | control 01 | 1 | | | | |
| Linaceae | Linum | perenne | Acetate 03 | 1 | | | | |
| Linaceae | Linum | usitatissimum | control 01 | | | | | |
| Linaceae | Linum | usitatissimum | Acetate 03 | | | | | |
| Linaceae | Linum | usitatissimum | control 01 | | | | | |
| Linaceae | Linum | usitatissimum | Acetate 03 | 3 | | | | |
| Loasaceae | Kissenia | capensis | control 01 | | | | | |
| Loasaceae | Kissenia | capensis | Acetate 03 | 2 | | | | |
| Loganiaceae | Buddleia | davidii | control 01 | | | | | |
| Loganiaceae | Buddleia | davidii | Acetate 03 | 3 | | | | |
| Loganiaceae | Strychnos | spinosa | control 01 | | | | | |
| Loganiaceae | Strychnos | spinosa | Acetate 03 | 4 | | | | |
| Magnoliaceae | Liriodendron | tulipifera | control 01 | | | | | |
| Magnoliaceae | Liriodendron | tulipifera | Acetate 03 | 1 | | | | |
| Malvaceae | Abutilon | sp. | control 01 | | | | | |
| Malvaceae | Abutilon | sp. | Acetate 03 | 5 | | | | |
| Malvaceae | Althaea | officinalis | control 01 | | | | | |
| Malvaceae | Althaea | officinalis | Acetate 03 | | | | | |
| Malvaceae | Althaea | officinalis | control 01 | | | | | |
| Malvaceae | Althaea | officinalis | Acetate 03 | 2, 0, 0 | | 0, 0, 3 | | |
| Malvaceae | Hibiscus | sabdariffa | control 01 | | | | | |
| Malvaceae | Hibiscus | sabdariffa | Acetate 03 | | 1, 0, 0 | 0, 0, 3 | | |
| Malvaceae | Lavatera | trimestris | control 01 | | | | | |
| Malvaceae | Lavatera | trimestris | Acetate 03 | 5 | | | | |
| Malvaceae | Malva | mauritanica | control 01 | 3 | | | | |
| Malvaceae | Malva | mauritanica | Acetate 03 | 3 | | | | |
| Malvaceae | Malva | moschata | control 01 | | | | | |
| Malvaceae | Malva | moschata | Acetate 03 | 5 | 2 | | | |
| Melianthaceae | Melianthus | villosua | control 01 | | | | | |
| Melianthaceae | Melianthus | villosua | Acetate 03 | 4 | | | | |
| Molluginaceae | Pharnaceum | sp. | control 01 | | | 5 | | |
| Molluginaceae | Pharnaceum | sp. | Acetate 03 | | | 5 | | |
| Montiniaceae | Montinia | caryophyllaceae | control 01 | | | | | |
| Montiniaceae | Montinia | caryophyllaceae | Acetate 03 | 2 | | 3 | | |
| Moraceae | Maclura | pomifera | control 01 | 3, 3, 3 | | | | |
| Moraceae | Maclura | pomifera | Acetate 03 | 3, 3, 3 | | | | |
| Moraceae | Morus | alba | control 01 | 4 | | | | |
| Moraceae | Morus | alba | Acetate 03 | 4 | | | | |
| Moraceae | Morus | alba | control 01 | 5 | | | 3, 0 | |
| Moraceae | Morus | alba | Acetate 03 | 5 | | | 3, 0 | |
| Moraceae | Morus | nigra | control 01 | 2, 0, 2 | | | | |
| Moraceae | Morus | nigra | Acetate 03 | 2, 0, 2 | | | | |
| Nyctaginaceae | Abronia | nana | control 01 | 3, 3, 3 | | 4, 0, 4 | | |
| Nyctaginaceae | Abronia | nana | Acetate 03 | 3, 3, 3 | | | | |
| Nyctaginaceae | Abronia | nana | Acetate 03 | 4, 2, 3 | | 2, 0, 0 | | |
| Oleaceae | Ligustrum | lucidum | control 01 | 2, 1 | | 2, 0 | | |
| Oleaceae | Ligustrum | lucidum | Acetate 03 | | 2, 0 | | | |
| Oleaceae | Ligustrum | sinense | control 01 | | | | | |
| Oleaceae | Ligustrum | sinense | Acetate 03 | | | | | |
| Oleaceae | Ligustrum | sinense | control 01 | | | | | |
| Oleaceae | Ligustrum | sinense | Acetate 03 | | | 3 | | |
| Oleaceae | Olea | europaea | control 01 | | 2, 0 | | | |
| Oleaceae | Olea | europaea | Acetate 03 | 2, 2 | 2, 2 | | | 2, 0 |
| Onagraceae | Camissonia | cheiranthifolia | control 01 | | | | | |
| Onagraceae | Camissonia | cheiranthifolia | Acetate 03 | | | | | |
| Onagraceae | Camissonia | cheiranthifolia | control 01 | | 3, 0, 0 | | | |
| Onagraceae | Camissonia | cheiranthifolia | Acetate 03 | | 3, 0, 0 | | | |
| Onagraceae | Clarkia | amoena | control 01 | | | | | |
| Onagraceae | Clarkia | amoena | Acetate 03 | | 1, 1, 0 | 2, 0, 0 | | |
| Onagraceae | Clarkia | amoena | control 01 | | | | | |
| Onagraceae | Clarkia | amoena | Acetate 03 | 3 | | 5 | | |
| Onagraceae | Clarkia | unguiculata | control 01 | | | | | |
| Onagraceae | Clarkia | unguiculata | Acetate 03 | 4 | | 4 | | |
| Onagraceae | Oenothera | lamarckianna | control 01 | 2, 0, 0 | 1, 0, 0 | | | |
| Onagraceae | Oenothera | lamarckianna | Acetate 03 | 2, 0, 0 | 1, 0, 0 | | | |
| Papaveraceae | Chelidonium | majus | control 01 | 3, 2, 0 | | 0, 0, 2 | | |
| Papaveraceae | Chelidonium | majus | Acetate 03 | 3, 1, 3 | | 0, 0, 2 | | |
| Papaveraceae | Eschscholzia | californica | control 01 | | | | | |
| Papaveraceae | Eschscholzia | californica | control 01 | | | | | |
| Papaveraceae | Eschscholzia | californica | control 01 | | | | | |
| Papaveraceae | Eschscholzia | californica | Acetate 02 | | | | | |
| Papaveraceae | Eschscholzia | californica | Acetate 02 | | | | | |
| Papaveraceae | Eschscholzia | californica | Acetate 02 | | | | | |
| Papaveraceae | Eschscholzia | californica | control 01 | 4 | | 5 | 2, 3 | |
| Papaveraceae | Eschscholzia | californica | Acetate 03 | 4 | | 5 | 2, 3 | |
| Papaveraceae | Eschscholzia | californica | control 01 | 4 | | 3 | | |
| Papaveraceae | Eschscholzia | californica | Acetate 03 | 5 | 2 | 3 | | |
| Papaveraceae | Eschscholzia | californica | control 01 | 4 | | | | |
| Papaveraceae | Eschscholzia | californica | Acetate 03 | 5 | | | | |
| Papaveraceae | Eschscholzia | californica | control 01 | 5 | | 5 | | |
| Papaveraceae | Eschscholzia | californica | Acetate 03 | 5 | | 5 | 5, 3 | |
| Papaveraceae | Papaver | rhoeas | control 01 | | | | | |
| Papaveraceae | Papaver | rhoeas | Acetate 03 | 4 | | | | |
| Pedaliaceae | Dicerocaryum | eriocarpum | control 01 | | | | | |
| Pedaliaceae | Dicerocaryum | eriocarpum | Acetate 03 | 5 | | | | |
| Pedaliaceae | Sesamum | indicum | control 01 | | | | | |
| Pedaliaceae | Sesamum | indicum | Acetate 03 | 2 | | | | |
| Penaeaceae | Penaea | cneorum | control 01 | 3 | | | | |
| Penaeaceae | Penaea | cneorum | Acetate 03 | 3 | | | | |
| Pinaceae | Larix | olgensis | control 01 | 5 | | 3 | | |
| Pinaceae | Larix | olgensis | Acetate 03 | 4 | | | 4, 3 | |
| Pinaceae | Pseudotsuga | glauca | control 01 | 1, 1 | | | 1, 0 | |
| Pinaceae | Pseudotsuga | glauca | Acetate 03 | 2, 3 | | | | 2, 0 |
| Pittosporaceae | Pittosporum | tobira | control 01 | 1 | | 1, 0, 0 | | |
| Pittosporaceae | Pittosporum | tobira | Acetate 03 | 1 | | 1, 0, 5 | | |
| Pittosporaceae | Pittosporum | viridiflorum | control 01 | | | | | |
| Pittosporaceae | Pittosporum | viridiflorum | Acetate 03 | | | | | |
| Pittosporaceae | Pittosporum | viridiflorum | control 01 | 4 | | 5 | | |
| Pittosporaceae | Pittosporum | viridiflorum | Acetate 03 | 4 | | 5 | | |
| Pittosporaceae | Sollya | heterophylla | control 01 | 4, 4 | | | 2, 0 | |
| Pittosporaceae | Sollya | heterophylla | Acetate 03 | 2, 3c | 1, 2 | 3, 2 | 3, 3 | 3, 0 |
| Plantaginaceae | Plantago | insularis | control 01 | 2, 0, 0 | 1, 0, 0 | | | |
| Plantaginaceae | Plantago | insularis | Acetate 03 | | 1, 0, 0 | | | |
| Plantaginaceae | Plantago | major | control 01 | | | | | |
| Plantaginaceae | Plantago | major | Acetate 03 | | | | | |
| Plantaginaceae | Plantago | major | control 01 | 1, 0 | | | | |
| Plantaginaceae | Plantago | major | Acetate 03 | | | | 2, 0 | |
| Polemoniaceae | Cobaea | scandens | control 01 | | | 5 | | |
| Polemoniaceae | Cobaea | scandens | Acetate 03 | | | 5 | | |
| Polemoniaceae | Gilia | aggregata | control 01 | | | 5, 5, 5 | | |
| Polemoniaceae | Gilia | aggregata | Acetate 03 | | | 5, 5, 5 | | |
| Polemoniaceae | Gilia | capitata | control 01 | | | | | |
| Polemoniaceae | Gilia | capitata | Acetate 03 | 1, 0, 0 | 1, 0, 1 | | | |
| Polemoniaceae | Gilia | stenothyrsa | control 01 | | | 5, 5, 5 | | |
| Polemoniaceae | Gilia | stenothyrsa | Acetate 03 | | | 5, 5, 5 | | |
| Polemoniaceae | Gilia | tricolor | control 01 | | | | | |
| Polemoniaceae | Gilia | tricolor | Acetate 03 | | | | | |
| Polemoniaceae | Gilia | tricolor | control 01 | 2 | | | | |
| Polemoniaceae | Gilia | tricolor | Acetate 03 | 2 | | | | |
| Polemoniaceae | Linanthus | grandiflorus | control 01 | | | | | |
| Polemoniaceae | Linanthus | grandiflorus | Acetate 03 | | | | | |
| Polemoniaceae | Linanthus | grandiflorus | control 01 | 2, 0, 1 | 2, 1, 2 | | | |
| Polemoniaceae | Linanthus | grandiflorus | Acetate 03 | 2, 0, 1 | 2, 1, 2 | | | |
| Polemoniaceae | Polemonium | caeruleum | control 01 | | | 4, 0, 5 | | |
| Polemoniaceae | Polemonium | caeruleum | Acetate 03 | | | 3, 0, 5 | | |
| Polemoniaceae | Polemonium | caeruleum | control 01 | | | 5, 5, 5 | 5 | |
| Polemoniaceae | Polemonium | caeruleum | Acetate 03 | | | 5, 5, 5 | | |
| Polemoniaceae | Polemonium | cashmirianum | control 01 | | | | | |
| Polemoniaceae | Polemonium | cashmirianum | Acetate 03 | | | | 2, 0 | 2, 0 |
| Polemoniaceae | Polemonium | cashmirianum | control 01 | | | 5, 5 | | |
| Polemoniaceae | Polemonium | cashmirianum | Acetate 03 | 3, 2 | 3, 0 | 5, 5 | 2, 4 | 2, 0 |
| Ranunculaceae | Clematis | ligusticifolia | control 01 | | | 1, 2 | | 1, 5 |
| Ranunculaceae | Clematis | ligusticifolia | Acetate 03 | | | | 2, 2 | |
| Ranunculaceae | Clematis | vitalba | control 01 | 2 | 2 | 3 | 3, 4 | |
| Ranunculaceae | Clematis | vitalba | Acetate 03 | 4 | | | | |
| Ranunculaceae | Delphinium | ajacis | control 01 | 4 | | | 3 | |
| Ranunculaceae | Delphinium | ajacis | Acetate 03 | 4 | | | | |
| Restionaceae | Calopsis | paniculata | control 01 | | | | | |
| Restionaceae | Calopsis | paniculata | control 01 | | 2 | | | |
| Restionaceae | Calopsis | paniculata | Acetate 03 | | 2 | | | |
| Rhamnaceae | Ceanothus | cuneatus | control 01 | 0, 0, 3 | 4, 0, 0 | | | |
| Rhamnaceae | Ceanothus | cuneatus | Acetate 03 | 5, 0, 4 | | | | |
| Rhamnaceae | Ceanothus | integerrimus | control 01 | 5 | 5 | | | |
| Rhamnaceae | Ceanothus | integerrimus | Acetate 03 | 5+ | | | | |
| Rhamnaceae | Hovenia | dulcis | control 01 | 4, 4 | | | 2, 2 | 1, 2 |
| Rhamnaceae | Hovenia | dulcis | Acetate 03 | 3, 3 | | 2, 0 | | 2, 0 |
| Rhamnaceae | Paliurus | spina-christi | control 01 | | | | | |
| Rhamnaceae | Paliurus | spina-christi | Acetate 03 | 1, 1, 0 | | | | |
| Rhamnaceae | Rhamnus | ilicifolia | control 01 | | | | | 2 |
| Rhamnaceae | Rhamnus | ilicifolia | Acetate 03 | 2 | | | | |
| Rhamnaceae | Ziziphus | jujuba | control 01 | 4 | | | | |
| Rhamnaceae | Ziziphus | jujuba | Acetate 03 | 4 | | | | |
| Rhamnaceae | Ziziphus | spina-christi | control 01 | 4 | | | | |
| Rhamnaceae | Ziziphus | spina-christi | Acetate 03 | 4 | 5 | | | |
| Rosaceae | Agrimonia | eupatoria | control 01 | | | | | |
| Rosaceae | Agrimonia | eupatoria | Acetate 03 | 2, 0, 0 | | | | |
| Rosaceae | Amelanchier | ovalis | control 01 | | | | | |
| Rosaceae | Amelanchier | ovalis | Acetate 03 | | | 2, 1 | | |
| Rosaceae | Amelanchier | utahensis | control 01 | | | 2, 0 | | |
| Rosaceae | Amelanchier | utahensis | Acetate 03 | 2, 1 | 3, 2 | 2, 2 | 1, 1 | 1, 0 |
| Rosaceae | Aruncus | dioicus | control 01 | 2, 0, 0 | 2, 1, 2 | 2, 0, 0 | | |
| Rosaceae | Aruncus | dioicus | Acetate 03 | 2, 0, 0 | 2, 1, 2 | 2, 0, 0 | | |
| Rosaceae | Crataegus | arnoldiana | control 01 | 2, 2 | | | | 3, 0 |
| Rosaceae | Crataegus | arnoldiana | Acetate 03 | | 1, 0 | 3, 0 | | 2, 0 |
| Rosaceae | Crataegus | coccinioides | control 01 | | | 2, 0 | | 1, 0 |
| Rosaceae | Crataegus | coccinioides | Acetate 03 | | | 2, 0 | | 1, 1 |
| Rosaceae | Geum | faurieri | control 01 | 5, 0, 0 | | | | |
| Rosaceae | Geum | faurieri | Acetate 03 | 3, 0, 0 | 4, 0, 0 | | | |
| Rosaceae | Holodiscus | discolor | control 01 | | | | | |
| Rosaceae | Holodiscus | discolor | Acetate 03 | | | | | |
| Rosaceae | Holodiscus | discolor | control 01 | | | | | |
| Rosaceae | Holodiscus | discolor | Acetate 03 | 2, 2 | 1, 2 | | 2, 3 | |
| Rosaceae | Prinsepia | sinensis | control 01 | | | | | |
| Rosaceae | Prinsepia | sinensis | Acetate 03 | | | | | |
| Rosaceae | Prinsepia | sinensis | control 01 | 3 | | | | |
| Rosaceae | Prinsepia | sinensis | Acetate 03 | 3 | | | | |
| Rosaceae | Prinsepia | uniflora | control 01 | | 1, 0 | | | |
| Rosaceae | Prinsepia | uniflora | Acetate 03 | | 1, 0 | | 1, 0 | |
| Rosaceae | Prunus | lyonii | control 01 | | 2, 0 | 2, 0 | | |
| Rosaceae | Prunus | lyonii | Acetate 03 | | 1, 0 | 3, 2 | | |
| Rosaceae | Pyrus | communis | control 01 | | 1, 1 | | | |
| Rosaceae | Pyrus | communis | Acetate 03 | | 1, 2 | | 1, 0 | |
| Rosaceae | Pyrus | communis | Acetate 03 | 1, 2 | | | | |
| Rosaceae | Rosa | canina | control 01 | | | | 2, 0 | |
| Rosaceae | Rosa | canina | Acetate 03 | | | | | 2, 0 |
| Rosaceae | Rosa | roxburghii | control 01 | 2c, 2 | | | 2, 2 | |
| Rosaceae | Rosa | roxburghii | Acetate 03 | 2c, 2 | | | 2, 3 | 2, 2 |
| Rutaceae | Ptelea | trifoliata | control 01 | | | | | |
| Rutaceae | Ptelea | trifoliata | Acetate 03 | 4 | | | | |
| Rutaceae | Ruta | graveolens | control 01 | 5 | | 3 | | |
| Rutaceae | Ruta | graveolens | Acetate 03 | 5 | | 3 | | |
| Sapindaceae | Sapindus | mukorossi | control 01 | | | | | |
| Sapindaceae | Sapindus | mukorossi | Acetate 03 | | 1, 1 | | | |
| Sapindaceae | Xanthocerus | sorbifolius | control 01 | 3 | | 5 | | |
| Sapindaceae | Xanthocerus | sorbifolius | Acetate 03 | 3 | 5 | | | |
| Sapotaceae | Sideroxylon | inerme | control 01 | | | | | |
| Sapotaceae | Sideroxylon | inerme | Acetate 03 | | 2 | | | |
| Saxifragaceae | Astilbe | chinensis | control 01 | | | | | |
| Saxifragaceae | Astilbe | chinensis | Acetate 03 | 2 | | 2 | | |
| Saxifragaceae | Heuchera | sanguinea | control 01 | 3, 2 | 1, 0 | 3, 3 | | |
| Saxifragaceae | Heuchera | sanguinea | Acetate 03 | 3, 2 | 1, 2 | 4, 4 | 2, 2 | |
| Saxifragaceae | Heuchera | sanguinea | control 01 | 3, 2 | 2, 0 | | | |
| Saxifragaceae | Heuchera | sanguinea | Acetate 03 | 3, 2 | 2, 2 | | | 2, 2 |
| Scrophulariaceae | Digitalis | grandiflora | control 01 | | | | | |
| Scrophulariaceae | Digitalis | grandiflora | Acetate 03 | | | 2, 0, 0 | | |
| Scrophulariaceae | Linaria | maroccana | control 01 | | | | | |
| Scrophulariaceae | Linaria | maroccana | Acetate 03 | 2 | | | | |
| Scrophulariaceae | Linaria | vulgaris | control 01 | | | | | |
| Scrophulariaceae | Linaria | vulgaris | Acetate 03 | | | 1, 0, 1 | | |
| Scrophulariaceae | Paulownia | tomentosa | control 01 | | | | | |
| Scrophulariaceae | Paulownia | tomentosa | Acetate 03 | 2 | | | | |
| Scrophulariaceae | Veronica | officinalis | control 01 | 2 | | | | |
| Scrophulariaceae | Veronica | officinalis | Acetate 03 | 2 | | | | |
| Solanaceae | Datura | stramonium | control 01 | | | | | |
| Solanaceae | Datura | stramonium | control 01 | | | | | |
| Solanaceae | Datura | stramonium | control 01 | | | | | |
| Solanaceae | Datura | stramonium | Acetate 01 | | | | | |
| Solanaceae | Datura | stramonium | Acetate 01 | | | | | |
| Solanaceae | Datura | stramonium | Acetate 01 | | | | | |
| Solanaceae | Datura | stramonium | control 01 | | | | | |
| Solanaceae | Datura | stramonium | Acetate 03 | | 1, 0, 0 | | | |
| Solanaceae | Hyoscyamus | albus | control 01 | 2, 2 | 2, 1 | 2, 2 | | 3, 2 |
| Solanaceae | Hyoscyamus | albus | Acetate 03 | 3, 3 | 2, 0 | 2, 2 | | 2, 2 |
| Solanaceae | Nicandra | physaloides | control 01 | 1, 0, 1 | 1, 0, 0 | | | |
| Solanaceae | Nicandra | physaloides | Acetate 03 | 1, 0, 1 | 1, 0, 1 | | | |
| Solanaceae | Petunia | hybrida | control 01 | 2, 2 | | | | |
| Solanaceae | Petunia | hybrida | Acetate 03 | 2, 2 | | | 2, 2 | 2, 3 |
| Styracaceae | Styrax | obassia | control 01 | | | 5 | | |
| Styracaceae | Styrax | obassia | Acetate 03 | 1 | 5 | | | |
| Theaceae | Camellia | japonica | control 01 | | | 5, 5, 5 | | |
| Theaceae | Camellia | japonica | Acetate 03 | | | 5, 5, 5 | | |
| Tropaeolaceae | Tropaeolum | peregrinum | control 01 | | | | | |
| Tropaeolaceae | Tropaeolum | peregrinum | Acetate 03 | | | 5 | | |
| Ulmaceae | Ulmus | parvifolia | control 01 | 2, 0 | | 1, 3c | | 2c, 4 |
| Ulmaceae | Ulmus | parvifolia | Acetate 03 | 2, 2 | | 1, 2 | | 2c, 2 |
| Valerianaceae | Valeriana | officinalis | control 01 | | | | | |
| Valerianaceae | Valeriana | officinalis | Acetate 03 | 3, 0, 2 | | | | |
| Verbenaceae | Caryopteris | sp. | control 01 | 3, 1, 3 | | | | |
| Verbenaceae | Caryopteris | sp. | Acetate 03 | 3, 1, 3 | | | | |
| Verbenaceae | Gmelina | arborea | control 01 | | 3, 2 | | | |
| Verbenaceae | Gmelina | arborea | Acetate 03 | 2, 2 | 3, 1 | | | |
| Violaceae | Viola | odorata | control 01 | | | | | 3, 2 |
| Violaceae | Viola | odorata | Acetate 03 | 2, 2 | 1, 0 | 2, 0 | | |
| Zingiberaceae | Elettaria | cardamomum | control 01 | 1, 0, 0 | 1, 0, 0 | 3, 0, 0 | | |
| Zingiberaceae | Elettaria | cardamomum | Acetate 03 | 1, 0, 0 | 1, 1, 1 | | | |
| Zygophyllaceae | Tribulus | terrestris | control 01 | | | | | |
| Zygophyllaceae | Tribulus | terrestris | Acetate 03 | | | 1, 0, 0 | | |
| Zygophyllaceae | Tribulus | terrestris | control 01 | | | | | |
| Zygophyllaceae | Tribulus | terrestris | Acetate 03 | | | | | |

The anti-microbial data collected in Table 6 is presented in terms of a scale from 0 (no growth inhibition) to 5 (essentially complete growth inhibition). Results from independent trials of the same extract exposed to the same microbe are separated by commas. The data indicate that a variety of plants exhibit reproducible anti-microbial activity (anti-bacterial, anti-fungal, or both activities) following elicitation of the plants with acetic acid.

Of the many plant extracts subjected to the anti-microbial assay, the following plants yielded extracts having no detectable anti-microbial activity, whether induced by acetic acid or not: Baphicacanthus cusia, Actinidia chinensis, Beaucamea recurvata, Aloinopsis luckhoffii, Aloinopsis rubrolineata, Delosperma ashtonii, Ruschia indurata, Ruschia pulvinaris, Tetragonia decumbens, Allium cepa, Allium schoenoprasum, Allium tuberosum, Achyranthes bidentata, Amaranthus tricolor, Celosia cristata, Cyathula officinalis, Pistacia chinensis, Pistacia vera, Rhus ovata, Annona cherimola, Annona senegalensis, Asimina triloba, Anethum graveolens, Angelica polymorpha, Apium graveloens, Carum carvi, Carum copticum, Coriandrum sativum, Daucus carota, Heracleum sphondylium, Pimpinella anisum, Saposhnikovia divaricata, Carissa grandiflora, Rauvolfia caffra, Acorus calamus, Monstera deliciosa, Zantedeschia aethiopica, Zantedeschia albomaculata, Aralia racemosa, Fatsia japonica, Aristolochia clematis, Asclepias curassavica, Asclepias tuberosa, Protasparagus africanus, Gasteria acinacifolia, Trachyandra divaricata, Trachyandra revoluta, Achillea ptarmica, Ageratum conyzoides, Antennaria dioica, Arctium lappa, Arnica chamissonis, Artemisia annua, Artemisia apiacea, Aster chilensis, Aster novae-angliae, Athrixia elata, Bellis perennis, Calendula officinalis, Carlina acaulis, Cichorium endivia, Cosmos sulphureus, Cynara cardunculus, Cynara scolymus, Echinacea angustifolia, Echinacea pallida, Echinacea purpurea, Eclipta alba, Erigeron glaucus, Eriophyllum confertiflorum, Grindelia robusta, Helenium autunmale, Helianthus annuus, Hieracium pilosella, Inula helenium, Lactuca sativa, Layia platyglossa, Leontopodium alpinum, Rudbeckia hirta, Schoenia cassiniana, Serratula tinctoria, Silphium laciniatum, Spilanthes oleracea, Stokesia laevis, Tagetes minuta, Taraxacum officinale, Telekia speciosa, Trichostemma lanatum, Tussilago farfara, Xanthium sibiricum, Impatiens balsamina, Begonia sutherlandii, Berberis julianae, Berberis poiretii, Alnus japonica, Betula nigra, Betula pendula, Carpinus betulus, Corylus avellana, Jacaranda mimosifolia, Adansonia digitata, Borago officinalis, Cynoglossum firmament, Echium vulgare, Heliotropium arborscens, Lithospermum officinale, Symphytum officinalis, Barbarea verna, Brassica campestris, Brassica nigra, Cochlearia officinalis, Crambe aspera, Crambe tataria, Hesperis matronalis, Iberis amara, Iberis intermedia, Isatis indigotica, Lepidium sativum, Lunaria annua, Raphanus sativus, Berzelia abrotanoides, Brunia albiflora, Brunia nodiflora, Opuntia humifusa, Calycanthus floridus, Chimonanthus praecox, Campanula rapunculus, Campanula trachelium, Platycodon grandiflorus, Wahlenbergia undulata, Cannabis sativa, Capparis inermis, Cleome spinosa, Kolkwitzia amabilis, Lonicera chamissoi, Lonicera maackii, Lonicera tatarica, Sambucus mexicana, Sambucus nigra, Viburnum prunifolium, Viburnum trilobum, Weigela florida, Carica papaya, Cerastium tomentosum, Dianthus andrzejowskianus, Dianthus barbatus, Dianthus gratianopolitanus, Lychnis coriacea, Sagina subulata, Saponaria officinalis, Stellaria media, Casuarina cunninghamiana, Casuarina equisetifolia, Maytenus capitata, Atriplex hortensis, Beta vulgaris, Chenopodium ambrosiodes, Chenopodium nuttaliae, Cistus incanus, Cistus ladanifer, Helianthemum grandiflorum, Hypericum montanum, Hypericum olympicum, Androcymbium pulherum, Gloriosa carsonii, Gloriosa rothschildiana, Terminalia brachystemma, Terminalia mantaly, Commelina communis, Pollia japonica, Tradescantia virginiana, Convolvulus cantabrica, Ipomoea purpurea, lpomoca tricolor, Pharbitis nil, Camptotheca acuminata, Nyssa aquatica, Sedum pulchellum, Sedum telephium, Bryonia alba, Citrullus colorynthis, Cucumis melo, Cucumis metuliferus, Cucurbita pepo, Lagenaria siceraria, Luffa aegyptiaca, Momordica charantia, Trichosanthes kirilowii, Cunonia capensis, Calocedrus decurreus, Cupressus lusitanica, Cupressus sempervirens, Thuja occidentalis, Cladium mariscus, Cyperus esculentus, Scirpus californicus, Scirpus robustus, Dioscorea batatas, Dioscorea dregeana, Dipsacus fullonum, Dipsacus sativus, Knautia arvensis, Scabiosa caucasica, Scabiosa lucida, Succisa pratensis, Dracaena hookeriana, Diospyros virginiana, Elaeagnus angustifolia, Ephedra nevadensis, Ephedra viridis, Croton californicus, Euphorbia lathyrsis, Ricinus communis, Securinega suffruticosa, Euptelea pleiosperma, Abrus precatorius, Acacia saligna, Albizia lebbeck, Arachis hypogaea, Astragalus membranaceus, Astragalus sinicus, Baptisia australis, Cajanus cajan, Cassia abbreviata, Chamaecrista fasciculata, Colutea arborescens, Cytissus albus, Dolichos biflorus, Erythrina christa-galli, Erythrina flabelliformis, Galega officinalis, Genista tinctoria, Glycine max, Glycyrrhiza echinata, Glycyrrhiza glabra, Glycyrrhiza glabra (echinata?), Indigofera suffruticosa, Indigofera tinctoria, Lablab purpureus, Lens culinaris?, Lespedeza capitata, Lupinus arboreus, Lupinus luteus, Lupinus nanus, Lupinus perennis, Lupinus polyphyllus, Lupinus propinquus, Medicago sativa, Petalostemon purpureum, Phaseolus mungo, Prosopis glandulosa, Psoralea pinnata, Pterocarpus indicum, Pueraria thunbergiana, Robinia pseudoacacia, Tamarindus indica, Tephrosia grandiflora, Tephrosia vogeiji, Trifolium pratense, Trifolium sp., Trigonella foenum-graecum, Vigna unguiculata, Wisteria sinensis, Castanea dentata, Scolopia zeyheri, Centaurium erythraea, Gentiana lutea, Geranium maculatum, Geranium sibiricum, Ginkgo biloba, Globularia aphyllanthesa, Globularia trichosanthes, Albuca altissima, Lachenalia pusilla, Philadelphus incanus, Nemophila menziesii, Phacelia campanularia, Belamcanda chinensis, Gladiolus cunonius, Iris germanica, Iris notha, Iris pseudoacorus, Iris sibirica, Carya aquatica, Juglans nigra, Juglans regia, Juncus acutus, Acinos thymoides, Agastache foeniculum, Agastache mexicana, Agastache nepetoides, Agastache rugosa, Ballota nigra, Calamintha nepeta, Clinopodium vulgare, Dracocephalum moldavica, Dracocephalum scrobiculatum, Elsholtzia stauntonii, Glechoma hederacea, Hyssopus officinalis, Lavandula officinalis, Leonotis nepetifolia, Leonurus sibiricus, Lycopus europaeus, Lycopus europeaus, Marrubium incanum, Marrubium vulgare, Mentha spicata, Monarda didyma, Monarda fistulosa, Nepeta cataria, Ocimum basilicum, Ocimum gratissimum, Origanum hirtum, Perilla frutescens, Prunella vulgaris, Pycnanthemum pycnanthemoides, Rosmarinus officinalis, Salvia apiana, Salvia columbariae, Salvia leucophylla, Salvia officinalis, Salvia sclarea, Satureja hortensis, Scutellaria altissima, Scutellaria creticola, Scutellaria orientalis, Sideritis syriaca, Stachys byzantina, Teucrium chamaedrys, Teucrium scorodonia, Thymus cretaceus, Thymus vulgaris, Trichostema lanatum, Lanaria lanata, Stauntonia hexaphylla, Cinnamomum camphora, Laurus nobilis, Lilium martagon, Mentzelia lindleyi, Galpinia transvaalica, Lagerstroemia indica, Lawsonia inermis, Magnolia grandiflora, Acridocarpus natalitius, Alcea rosea, Althaea broussonetifolia, Althaea cannabina, Gossypium herbaceum, Hibiscus trionum, Malacothamnus fasciculatus, Dissotis senegambiensis, Pseudosbeckia swynnertonii, Entandrophragma caudatum, Toona serrata, Melianthus major, Ficus religiosa, Morina longifolia, Myrica pensylvanica, Callistemon citrinus, Melaleuca altemifolia, Myrthus communis, Psidium guajava, Abronia villosa, Mirabilis jalapa, Forsythia suspensa, Fraxinus excelsior, Fraxinus pennsylvanica, Maytenus capitata, Syringa villosa, Camissonia pallida, Clarkia rubicunda, Oenothera biennis, Oenothera speciosa, Paeonia suffruticosa, Argemone mexicana, Eschscholzia caespitosa, Papaver dubium, Papaver orientate, Passiflora caerulea, Passiflora edulis, Ceratotheca sesamoides, Harpagophytum procumbens, Harpagophytum zeyheri, Phytolacca americana, Abies balsamea, Abies nephrolepis, Cedrus atlantica, Cedrus deodara, Pinus pinea, Plantago asiatica, Plantago erecta, Plantago hirtella, Plantago sempervirens, Plantago squalida, Platanus occidentalis, Armeria juniperifolia, Arneria maritima, Limonium gmeinii, Bouteloua curtipendula, Cortaderia selloana, Cymbopogon flexuosus, Festuca heterophylla, Festuca rubra, Merxmuellera arundinacea, Panicum virgatum, Polypogon monspeliensis, Schizachyrium scoparium, Setaria sphacelata, Sorghum sudanense, Vetiveria zizanoides, Podocarpus falcatus, Eriastrum densifolium, Phlox drummondii, Phlox paniculata, Securidaca longepedunculata, Eriogonum latifolium, Eriogonum umbellum, Polygonum hydropiper, Polygonum lapathifolium, Polygonum odoratum, Polygonum orientate, Polygonum punctatum, Rheum altaicum, Rheum palmatum, Rumex acetosa, Rumex confertus, Rumex crispus, Rumex euxinus, Rumex obtusifolius, Calandrinia ciliata, Claytonia perfoliata, Portulacca oleracea, Anagallis arvensis, Lysimachia punctata, Primula elatior, Aconitum napellus, Adonis aestivalis, Anemone pulsatilla, Aquilegia formosa, Aquilegia transsilvanica, Aquilegia vulgaris, Clematis manschurica, Clematis virginiana, Consolida orientalis, Nigella arvensis, Nigella damascena, Nigella sativa, Thalictrum flavum, Trollius europaeus, Reseda luteola, Reseda odorata, Rhamnus cathartica, Agrimonia pilosa, Aruncus kamtschaticus, Cercocarpus betuloides, Chaenomeles japonica, Filipendula vulgaris, Geum urbanum, Pentaphylloides mandschurica, Physocarpus opulifolius, Potentilla recta, Potentilla tormentilla, Poterium sanguisorba, Pyracantha coccinea, Rosa damascena, Sanguisorba officinalis, Sorbaria arborea, Sorbaria sorbifolia, Cephalanthus occidentalis, Galium aparine, Galium spurium, Galium verum, Gardenia jasminoides, Rubia tinctorum, Evodia daniellii, Evodia hupehensis, Phellodendron amurense, Cardiospermum grandiflorum, Cardiospermum halicacabum, Koelreuteria paniculata, Litchi chinensis, Anemopsis califomica, Bergenia cordifolia, Bergenia crassifolia, Heuchera pilosissima, Antirrhinum majus-maximum, Castilleja exserta, Collinsia canadensis, Collinsia heterophylla, Digitalis lanata, Digitalis purpurea, Gratiola officinalis, Hebenstreitia fastigiosa, Mimulus guttatus, Mimulus puniceus, Penstemon barbatus, Scrophularia nodosa, Selago corymbosa, Verbascum thapsus, Veronica spicata, Ailanthus altissima, Kirkia acuminata, Simmondsia chinensis, Atropa bella-donna, Brugmansia suaevolens, Capsicum annuum, Capsicum chinense, Datura inoxia, Lycium barbatum, Lycopersicon esculentum, Physalis ixocarpa, Solanum aculeatissimum, Solanum dulcamara, Solanum melongena, Withania somnifera, Brachychiton discolor, Sterculia africana, Sterculia quinqueloba, Strelitzia nicolae, Strelitzia reginae, Cunninghamia lanceolata, Taxodium distichum, Grewia biloba, Grewia flavescens, Grewia pachycalyx, Tropaeolum majus, Typha latifolia, Celtis occidentalis, Ulmus americana, Ulmus rubra, Zelkova sinica, Urtica dioica, Valerianella locusta, Verbena hastata, Verbena officinalis, Verbena tenuisecta, Vitex agnus-castus, Cyphostemma juttae, Vitis amurensis, Vitis vinifera, Zingiber officinale, Balanites maughamii, Larrea tridentata, and Peganum harmala.

Additionally, the following plants responded to acetic acid by yielding extracts that inhibited an anti-microbial activity observed in the extracts of un-elicited plants: Ammi majus, Eryngium campestre, Hydrocotyle asiatica, Levisticum officinale, Pboenix reclinata, Bulbine aloides, Machaeranthera bigelovii, Parthenium integrifolium, Ratibida columnifera, Iberis pinnata, Trichocereus pachanoi, Campanula carpatica, Humulus lupulus, Hypericum androsaemum, Sedum spurium, Cupressus sargentii, Hippophaë rhamnoides, Ephedra nevadensis, Ephedra viridis, Oxydendrum arboreum, Eremocarpus setigerus, Acacia redolens, Cassia hebecarpa, Delonix regia, Lupinus densiflorus, Lupinus luteus, Lupinus nanus, Lupinus perennis, Lupinus polyphyllus, Lupinus propinquus, Lupinus succulentus, Mucuna deeringiana, Robinia fertilis, Phacelia bolanderi, Iris versicolor, Scutellaria baicalensis, Limnanthes douglasii, Convallaria majalis, Linum hirsutum, Alcea nigra, Toona sinensis, Melianthus comosus, Morus rubra, Morina longifolia, Myrica cerifera, Myrica pensylvanica, Callistemon citrinus, Leptospermum scoparium, Syringa amurensis, Syringa pekinensis, Syringa villosa, Syringa vulgaris, Gymnaglossum officinale, Papaver bracteatum, Proboscidea louisianica, Abies firma, Bauchea, Primula japonica, Grevillea robusta, Punica granatum, Paliurus hemsleyanus, Cydonia oblonga, Malus communis, Tellima grandiflora, Schisandra chinensis, Antirrhinum majus-maximum, Castilleja exserta, Chelone glabra, Vemonia noveboracensis, Hyoscyamus niger, Firmiana simplex, Fremontodendron califomicum, Cryptomeria japonica, Camellia sinensis, Vitex negundo, Vitis labrusca, Vitis vinifera, Welwitschia mirabilis, and Cnidium monnieri.

While the data described above indicate that some plants do not contain detectable levels of an anti-microbial activity, and others show activity both with and without elicitation, it is significant that approximately 40% of all active plant species exhibited activity only after elicitation with acetic acid. Without wishing to be bound by theory, the data suggest that new biosynthetic pathways are being triggered, resulting in the accumulation of novel biologically active substances and/or activation of production pathways for existing biologically active substances is occurring, resulting in pronounced antimicrobial activities, and therefore in potential new drug development. Using the growth regimens described herein, e.g., hydroponic plant growth under controlled nutrient and light conditions, the results have been shown to be highly reproducible (approximately 80% of all trials with a given plant yield consistent outcomes in terms of anti-microbial activity), in contrast to the variations typically seen when investigating plant material collected following traditional protocols in drug discovery programs from natural sources.

One of ordinary skill in the art will recognize that many variations of the above-described bioassay exist and are suitable for testing extracts of plants elicited with acetic acid for anti-microbial activity. For example, anti-bacterial bioassays may be conducted by delivering extracts to MDS Pharma Services, Taipei, Taiwan, for testing in its standard anti-microbial screening assay.

### Example 9

### Anti-cancer Activity Elicited by Acetic Acid

The anti-cancer activity of extracts from control and elicited plants was assessed through collaboration with the National Cancer Institute (NCI). Extracts of plant roots were generally prepared according to the protocol described in Example 4. In particular, plant roots (approximately 1-5 g fresh weight) were harvested and freeze-dried, followed by an extraction with 20 ml of 80% (v/v) methanol per gram of lyophilized roots at room temperature for 48 hours. The methanol-root compositions are then centrifuged and the supernatant is decanted. The extract is formed by drying the supernatant by evaporation. Typically, 1-2 mg of extract were used in each bioassay.

The anti-cancer assays were conducted using a panel of three cancer cell lines, breast (NCI line MCF-7), melanoma (NCI line UACC-62), and renal (NCI line TK-10) cancer cell lines, or breast (NCI line MCF-7), central nervous system (NCI line SF-268), and non-small cell lung (NCI line NCI-H460) cancer cell lines. [Do we have to worry about biological deposits and ATCC information here?] A single-dose, 48-hour continuous exposure protocol was used and a sulforhodamine B (SRB) protein assay was used to estimate cancer cell growth. Anti-cancer activities have been expressed as percent growth inhibition. The numbers in the cells representing the detected anti-cancer activity are percentage growth of the corresponding cancer cells, calculated according to one of the following equations:

$$\frac{100 x ( MeanODtest - MeanODtzero )}{( MeanODctrl - MeanODtzero}$$

$$If ( MeanODTest - MeanODtzer ) \geq 0 , then PG = , \\ \frac{100 x ( MeanODtest - MeanODtzero )}{( MeanODtzero )}$$

$$or if ( MeanODtest - MeanODtzero ) < 0 , then PG = ,$$

If (MeanODtest—MeanODtzero)>0, then PG=,

or if (MeanODtest—MeanODtzero)<0, then PG=,

where:

PG is percent growth;

MeanODtzero is the average of two optical density (OD) measurements of SRB-derived color in a cell culture just before exposure of the cancer cells to the plant extract;

MeanODtest is the average of two OD measurements of SRB-derived color in a cell culture after 48 hours exposure of the cancer cells to the plant extract; and

MeanODctrl is the average of two OD measurements of SRB-derived color in a cell culture after 48 hours with no exposure of cancer cells to plant extract.

The plant species screened for anti-cancer activity are identified in Table 7, below.

TABLE 7
Anti-cancer activity of extracts elicited with acetic acid

| Family | Genus | species | treatment | Breast | Melanoma | Renal | CNS | Lung |
|---|---|---|---|---|---|---|---|---|
| Agavaceae | Agave | chrysantha | control 01 | | | | | |
| Agavaceae | Agave | chrysantha | Acetate 03 | −37 | | | −14 | −49 |
| Anacardiaceae | Rhus | integrifolia | control 01 | | | | | |
| Anacardiaceae | Rhus | integrifolia | Acetate 03 | −13 | | | −15 | |
| Anacardiaceae | Rhus | ovata | control 01 | | | | | |
| Anacardiaceae | Rhus | ovata | Acetate 03 | −24 | | | −25 | −20 |
| Anacardiaceae | Rhus | typhina | control 01 | | | | | |
| Anacardiaceae | Rhus | typhina | Acetate 03 | −62 | | | −68 | −47 |
| Asteraceae | Anthemis | nobilis | control 01 | | | | | |
| Asteraceae | Anthemis | nobilis | Acetate 03 | −47 | | | | −71 |
| Asteraceae | Bellis | perennis | control 01 | | | | | |
| Asteraceae | Bellis | perennis | Acetate 03 | −79 | | | −84 | −89 |
| Asteraceae | Calendula | officinalis | control 01 | | | | | |
| Asteraceae | Calendula | officinalis | Acetate 03 | −38 | | | −51 | |
| Asteraceae | Chrysanthemum | coronarium | control 01 | | | | | |
| Asteraceae | Chrysanthemum | coronarium | Acetate 03 | −25 | | | | |
| Asteraceae | Chrysanthemum | coronarium | control 01 | | | | | |
| Asteraceae | Chrysanthemum | coronarium | Acetate 03 | | | | −69 | −60 |
| Asteraceae | Chrysanthemum | parthenium | control 01 | | | | | |
| Asteraceae | Chrysanthemum | parthenium | Acetate 03 | | | | | −37 |
| Asteraceae | Lasthenia | glabrata | control 01 | | | | | |
| Asteraceae | Lasthenia | glabrata | Acetate 03 | | | | | −6 |
| Asteraceae | Layia | platyglossa | control 01 | | | | | |
| Asteraceae | Layia | platyglossa | Acetate 03 | −25 | | | | −79 |
| Asteraceae | Solidago | virgaurea | control 01 | | | | | |
| Asteraceae | Solidago | virgaurea | Acetate 03 | | | | | −62 |
| Asteraceae | Solidago | virgaurea | control 01 | | | | | |
| Berberidaceae | Berberis | thunbergii | control 01 | −50 | −57 | | | |
| Berberidaceae | Berberis | thunbergii | Acetate 03 | −5 | −31 | | | |
| Berberidaceae | Berberis | thunbergii | control 01 | | | | | |
| Berberidaceae | Berberis | thunbergii | Acetate 03 | | | | | |
| Betulaceae | Alnus | japonica | control 01 | | | | | |
| Betulaceae | Alnus | japonica | Acetate 03 | −49 | −22 | | | |
| Betulaceae | Alnus | japonica | control 01 | | | | | |
| Brassicaceae | Barbarea | verna | control 01 | | | | | |
| Brassicaceae | Barbarea | verna | Acetate 03 | | | | | −17 |
| Brassicaceae | Iberis | intermedia | control 01 | | | | −25 | |
| Brassicaceae | Iberis | intermedia | Acetate 03 | | | | −12 | −60 |
| Bruniaceae | Brunia | nodiflora | Acetate 03 | −6 | | | −2 | −24 |
| Caryophyllaceae | Dianthus | andrzejowskia | control 01 | | −93 | −97 | | |
| Caryophyllaceae | Dianthus | andrzejowskia | Acetate 03 | | −65 | −79 | | |
| Caryophyllaceae | Gypsophila | paniculata | control 01 | | | | | |
| Caryophyllaceae | Gypsophila | paniculata | Acetate 03 | −85 | | | −90 | −85 |
| Caryophyllaceae | Lychnis | chalcedonica | control 01 | | | | | |
| Caryophyllaceae | Lychnis | chalcedonica | Acetate 03 | −82 | | | −81 | −79 |
| Chenopodiaceae | Atriplex | hortensis | control 01 | | | | | |
| Chenopodiaceae | Atriplex | hortensis | Acetate 03 | −21 | −67 | | | |
| Chenopodiaceae | Atriplex | hortensis | control 01 | | | | | |
| Chenopodiaceae | Atriplex | hortensis | Acetate 03 | | | | | |
| Cucurbitaceae | Bryonia | alba | control 01 | | | | | |
| Cucurbitaceae | Bryonia | alba | Acetate 03 | −43 | | | −62 | −50 |
| Cucurbitaceae | Citrullus | colorynthis | control 01 | | | | −24 | |
| Cucurbitaceae | Citrullus | colorynthis | Acetate 03 | | | | −4 | |
| Cucurbitaceae | Cucumis | sativus | control 01 | | | | −26 | |
| Cucurbitaceae | Cucumis | sativus | Acetate 03 | | | | −13 | |
| Cucurbitaceae | Lagenaria | siceraria | control 01 | | | | −21 | |
| Cucurbitaceae | Lagenaria | siceraria | Acetate 03 | | | | −29 | −46 |
| Cucurbitaceae | Momordica | charantia | control 01 | | | | | |
| Cucurbitaceae | Momordica | charantia | Acetate 03 | −93 | | | −91 | −88 |
| Cucurbitaceae | Trichosanthes | kirilowii | control 01 | | | | | |
| Cucurbitaceae | Trichosanthes | kirilowii | Acetate 03 | | | −65 | | |
| Ebenaceae | Diospyros | kaki | control 01 | | | | | |
| Ebenaceae | Diospyros | kaki | Acetate 03 | −30 | −95 | −98 | | |
| Fabaceae | Albizia | julibrissin | control 01 | | | | −56 | −1 |
| Fabaceae | Albizia | julibrissin | Acetate 03 | | | | −62 | −30 |
| Fabaceae | Cytissus | albus | control 01 | | | | | |
| Fabaceae | Cytissus | albus | Acetate 03 | −18 | −50 | | | |
| Fabaceae | Cytissus | scoparius | control 01 | | | | | |
| Fabaceae | Cytissus | scoparius | Acetate 03 | −41 | −96 | −95 | | |
| Fabaceae | Genista | tinctoria | control 01 | | | | | |
| Faraceae | Genista | tinctoria | Acetate 03 | | −10 | | | |
| Fabaceae | Glycyrrhiza | echinata | control 01 | | | | | |
| Fabaceae | Glycyrrhiza | echinata | Acetate 03 | −35 | | | −29 | −15 |
| Fabaceae | Gymnocladus | diocus | control 01 | | | | | |
| Fabaceae | Gymnocladus | doicus | Acetate 03 | −28 | | | −96 | −62 |
| Fabaceae | Lupinus | polyphyllus | control 01 | | | | | |
| Fabaceae | Lupinus | polyphyllis | Acetate 03 | −27 | −48 | | | |
| Fabaceae | Pachyrhizus | erosus | control 01 | | | | | |
| Fabaceae | Pachyrhizus | erosus | Acetate 03 | −69 | | | −49 | −63 |
| Fabaceae | Schrankia | occidentalis | control 01 | | | | | |
| Fabaceae | Schrankia | occidentalis | Acetate 03 | −60 | −86 | −93 | | |
| Flacourtiaceae | Scolopia | zeyheri | control 01 | | | | | |
| Flacourtiaceae | Scolopia | zeyheri | Acetate 03 | | | | | −27 |
| Hippocastanacea | Aesculus | woerlitzensis | control 01 | −70 | −78 | −84 | | |
| Hippocastanacea | Aesculus | woerlitzensis | Acetate 03 | −51 | −78 | −88 | | |
| Juglandaceae | Juglans | regia | control 01 | | −45 | | | |
| Juglandaceae | Juglans | regia | Acetate 03 | −50 | −49 | | | |
| Lamiaceae | Dracocephalum | moldavica | control 01 | | | | | |
| Lamiaceae | Dracocephalum | moldavica | Acetate 03 | | | | | −3 |
| Lamiaceae | Lycopus | europaeus | control 01 | | | | | |
| Lamiaceae | Lycopus | europaeus | Acetate 03 | −1 | | | | |
| Lamiaceae | Marrubium | vulgare | control 01 | | | | | |
| Lamiaceae | Marrubium | vulgare | Acetate 03 | −45 | | | −66 | −65 |
| Lamiaceae | Marrubium | vulgare | control 01 | | | | | |
| Lamiaceae | Marrubium | vulgare | Acetate 03 | | | | | |
| Lamiaceae | Salvia | dumetorum | control 01 | | | | | |
| Lamiaceae | Salvia | dumetorum | Acetate 03 | −3 | −78 | −17 | | |
| Lamiaceae | Scutellaria | baicalensis | control 01 | | | | | |
| Lamiaceae | Scutellaria | baicalensis | Acetate 03 | | −16 | | | |
| Lamiaceae | Scutellaria | creticola | control 01 | | | | | |
| Lamiaceae | Scutellaria | creticola | Acetate 03 | | −18 | −10 | | |
| Linaceae | Linum | usitatissimum | control 01 | | | | | |
| Linaceae | Linum | usitatissimum | Acetate 03 | | | | | −52 |
| Loganiaceae | Buddleia | davidii | control 01 | | | | | |
| Loganiaceae | Buddleia | davidii | Acetate 03 | −47 | | | −12 | −71 |
| Malpighiaceae | Acridocarpus | natalitius | control 01 | | | | | |
| Malpighiaceae | Acridocarpus | natalitius | Acetate 03 | | | | | −5 |
| Malvaceae | Malva | moschata | control 01 | | | | | |
| Malvaceae | Malva | moschata | Acetate 03 | −9 | | | | −55 |
| Melastomataceae | Pseudosbeckia | swynnertonii | control 01 | | | | | |
| Melastomataceae | Pseudosbeckia | swynnertonii | Acetate 03 | −38 | | | | |
| Meliaceae | Entandrophragma | caudatum | control 01 | | | | | |
| Meliaceae | Entandrophragma | caudatum | Acetate 03 | | | | | −12 |
| Melianthaceae | Melianthus | comosus | control 01 | −51 | | | −67 | −53 |
| Melianthaceae | Melianthus | comosus | Acetate 03 | −26 | | | | −48 |
| Melianthaceae | Melianthus | villosua | control 01 | | | | | |
| Melianthaceae | Melianthus | villosua | Acetate 03 | | | | | −3 |
| Molluginaceae | Pharnaceum | sp. | control 01 | | | | | |
| Molluginaceae | Pharnaceum | sp. | Acetate 03 | −56 | | | −74 | −77 |
| Moraceae | Maclura | pomifera | control 01 | | −35 | | | |
| Moraceae | Maclura | pomifera | Acetate 03 | | −56 | | | |
| Papaveraceae | Chelidonium | majus | control 01 | | | −21 | | |
| Papaveraceae | Chelidonium | majus | Acetate 03 | −86 | −93 | −98 | | |
| Papaveraceae | Eschscholzia | californica | control 01 | | | | −22 | |
| Papaveraceae | Eschscholzia | californica | Acetate 03 | −36 | | | −66 | −47 |
| Pedaliaceae | Proboscidea | louisianica | control 01 | | | | | |
| Pedaliaceae | Proboscidea | louisianica | Acetate 03 | −43 | | | −16 | −72 |
| Pittosporaceae | Pittosporum | tobira | control 01 | | | | | |
| Pittosporaceae | Pittosporum | tobira | Acetate 03 | −63 | −81 | −97 | | |
| Pittosporaceae | Pittosporum | viridiflorum | control 01 | | | | | |
| Pittosporaceae | Pittosporum | viridiflorum | Acetate 03 | | | | | |
| Pittosporaceae | Pittosporum | viridiflorum | control 01 | | | | | |
| Pittosporaceae | Pittosporum | viridiflorum | Acetate 03 | −63 | | | −63 | −70 |
| Polemoniaceae | Eriastrum | densifolium | control 01 | | | −42 | | |
| Polemoniaceae | Eriastrum | densifolium | Acetate 03 | −57 | −81 | −70 | | |
| Polemoniaceae | Eriastrum | densifolium | control 01 | | | | | |
| Polemoniaceae | Gilia | aggregata | control 01 | −92 | | | −82 | −82 |
| Polemoniaceae | Gilia | aggregata | Acetate 03 | −91 | | | −77 | −84 |
| Polemoniaceae | Gilia | stenothyrsa | control 01 | | | | | |
| Polemoniaceae | Gilia | stenothyrsa | Acetate 03 | −59 | | | −59 | −78 |
| Polemoniaceae | Linanthus | grandiflorus | control 01 | | | | −73 | −93 |
| Polemoniaceae | Linanthus | grandiflorus | Acetate 03 | | | | −10 | −80 |
| Polemoniaceae | Linanthus | grandiflorus | control 01 | −36 | | | −98 | −87 |
| Polemoniaceae | Linanthus | grandiflorus | Acetate 03 | | | | −79 | −76 |
| Polemoniaceae | Polemonium | caeruleum | control 01 | | | | | |
| Polemoniaceae | Polemonium | caeruleum | Acetate 03 | | | | | |
| Polemoniaceae | Polemonium | caeruleum | control 01 | −84 | | | −73 | −80 |
| Polemoniaceae | Polemonium | caeruleum | Acetate 03 | −70 | | | −52 | |
| Polygonaceae | Polygonum | odoratum | control 01 | | | | | |
| Polygonaceae | Polygonum | odoratum | Acetate 03 | | | | | −37 |
| Polygonaceae | Polygonum | odoratum | control 01 | | | | | −14 |
| Polygonaceae | Polygonum | odoratum | Acetate 03 | | | | | |
| Primulaceae | Lysimachia | punctata | control 01 | | | | −35 | |
| Primulaceae | Lysimachia | punctata | Acetate 03 | −79 | | | −81 | −86 |
| Primulaceae | Primula | japonica | control 01 | | | | | |
| Primulaceae | Primula | japonica | Acetate 03 | −47 | −74 | −88 | | |
| Rosaceae | Agrimonia | pilosa | control 01 | | | | | |
| Rosaceae | Agrimonia | pilosa | control 01 | | | | | |
| Rosaceae | Agrimonia | pilosa | Acetate 03 | | −16 | | | |
| Rosaceae | Filipendula | vulgaris | control 01 | | | | | |
| Rosaceae | Filipendula | vulgaris | Acetate 03 | −71 | −52 | | | |
| Rosaceae | Filipendula | vulgaris | control 01 | | | | | |
| Rosaceae | Filipendula | vulgaris | Acetate 03 | | | | | |
| Saxifragaceae | Bergenia | crassifolia | control 01 | | | | | −43 |
| Saxifragaceae | Bergenia | crassifolia | Acetate 03 | | | | | −19 |
| Scrophulariaceae | Collinsia | heterophylla | control 01 | | | | | |
| Scrophulariaceae | Collinsia | heterophylla | Acetate 03 | | | | | −62 |
| Scrophulariaceae | Digitalis | grandiflora | control 01 | −58 | | | −88 | −84 |
| Scrophulariaceae | Digitalis | grandiflora | Acetate 03 | | | | −84 | −80 |
| Scrophulariaceae | Gratiola | officinalis | control 01 | | | −30 | | |
| Scrophulariaceae | Gratiola | officinalis | Acetate 03 | | | −51 | | |
| Tiliaceae | Grewia | pachycalyx | control 01 | | | | | |
| Tiliaceae | Grewia | pachycalyx | Acetate 03 | | | | | −8 |
| Valerianaceae | Valerianella | locusta | control 01 | | | | | |
| Valerianaceae | Valerianella | locusta | Acetate 03 | −4 | | | | |
| Verbenaceae | Verbena | tenuisecta | control 01 | | | | | |
| Verbenaceae | Verbena | tenuisecta | Acetate 03 | −48 | | | −46 | −58 |

The data indicate that a substantial number of plants exhibit anti-cancer activity following elicitation with 0.1% (v/v) acetic acid.

Several of the many plant extracts subjected to the anti-microbial assay yielded extracts having no detectable anti-cancer activity, whether induced by acetic acid or not, such as the following: Baphicacanthus cusia, Actinidia chinensis, Aloinopsis luckhoffii, Aloinopsis rubrolineata, Aloinopsis spathulata, Delosperma ashtonii, Ruschia indurata, Rusehia pulvinaris, Tetragonia decumbens, Allium ampeloprasum, Allium tuberosum, Achyranthes bidentata, Amaranthus retroflexus, Amaranthus tricolor, Celosia cristata, Malosma laurina, Schinus terebinthifolius, Asimina triloba, Ammi majus, Angelica polymorpha, Apium graveloens, Carum carvi, Carum copticum, Cnidium monnieri, Cryptotaenia japonica, Cuminum cyminum, Heracleum sphondylium, Hydrocotyle asiatica, Levisticum officinale, Ligusticum porteri, Petroselinum crispum, Pimpinella anisum, Saposhnikovia divaricata, Rauvolfia caffra, Monstera deliciosa, Zantedeschia albomaculata, Aralia racemosa, Fatsia japonica, Araucaria araucana, Phoenix reclinata, Protasparagus africanus, Bulbine aloides, Kniphofia baurii, Achillea ptarmica, Ageratum conyzoides, Antennaria dioica, Arctium lappa, Arnica chamissois, Artemisia absinthium, Artemisia apiacea, Aster novae-angliae, Carlina acaulis, Centaurea maculosa, Chrysanthemum leucanthemum, Cichorium endivia, Corethrogyne califomica, Cosmos sulphureus, Cynara cardunculus, Cynara scolymus, Eclipta alba, Eupatorium cannabinum, Eupatorium maculatum, Helenium autumnale, Inula ensifolia, Inula helenium, Lactuca sativa, Leontopodium alpinum, Liatris spicata, Pyrethrum corymbosum, Ratibida columnifera, Rudbeekia hirta, Serratula tinctoria, Silphium laciniatum, Silybum marianum, Spilanthes oleracea, Tagetes minuta, Taraxacum officinale, Telekia speciosa, Tussilago farfara, Xanthium sibiricum, Xanthium strumarium, Impatiens balsamina, Begonia sutherlandii, Nandina domestica, Podophyllum emodii, Podophyllum hexandrum, Betula nigra, Betula pendula, Corylus avellana, Chilopsis linearis, Adansonia digitata, Anchusa officinalis, Borago officinalis, Cynoglossum firmament, Heliotropium arborscens, Lithospermum officinale, Symphytum officinalis, Brassica campestris, Brassica hirta, Brassica nigra, Brassica oleracea, Brassica rapa, Crambe aspera, Hesperis matronalis, Isatis tinctoria, Lepidium sativum, Lunaria annua, Nasturtium officinale, Raphanus sativus, Berzelia abrotanoides, Calycanthus floridus, Chimonanthus praecox, Adenophora bulleyana, Campanula carpatica, Campanula persicifolia, Campanula rapunculus, Campanula trachelium, Codonopsis pilosula, Lobelia siphilitica, Platycodon grandiflorum, Platycodon grandiflorus, Humulus lupulus, Capparis inermis, Kolkwitzia amabilis, Lonicera chamissoi, Lonicera maackii, Lonicera tatarica, Sambucus nigra, Sambucus tigranii, Carica papaya, Cerastium tomentosum, Dianthus gratianopolitanus, Lychnis alba, Lychnis coriacea, Stellaria media, Maytenus capitata, Chenopodium ambrosiodes, Chenopodium botrys, Chenopodium nuttaliae, Cistus incanus, Cistus ladanifer, Fumana procumbens, Helianthemum grandiflorum, Hypericum androsaemum, Hypericum olympicum, Hypericum perforatum, Terminalia mantaly, Cyanotis speciosa, Convolvulus cantabrica, Ipomoea purpurea, Camptotheca acuminata, Nyssa aquatica, Sedum spurium, Sedum telephium, Cucumis metuliferus, Calocedrus decurreus, Cupressus lusitanica, Cupressus sempervirens, Thuja occidentalis, Thuja orientalis, Cladium mariscus, Cyperus esculentus, Scirpus californicus, Scirpus robustus, Dioscorea dregeana, Dipsacus fullonum, Dipsacus sativus, Knautia arvensis, Scabiosa caucasica, Scabiosa lucida, Succisa pratensis, Dracaena hookeriana, Diospyros virginiana, Elaeagnus commutata, Hippophaë rhamnoides, Ephedra nevadensis, Ephedra viridis, Eremocarpus setigerus, Euphorbia lathyrsis, Securinega suffruticosa, Euptelea pleiosperma, Abrus precatorius, Acacia redolens, Acacia saligna, Albizia kolomikta, Astragalus membranaceus, Astragalus sinicus, Cassia hebecarpa, Cassia tora, Ceratonia siliqua, Chamaecrista fasciculata, Cicer arietinum, Dalea candida, Erythrina christa-galli, Erythrina corallodendron, Galega officinalis, Glycyrrhiza echinata (glabra?), Glycyrrhiza glabra, Glycyrrhiza glabra (echinata?), Indigofera suffruticosa, Indigofera tinctoria, Lablab purpureus, Labumum anagyroides, Lespedeza capitata, Lupinus arboreus, Lupinus perennis, Lupinus propinquus, Medicago sativa, Ononis spinosa, Petalostemon candidum, Pterocarpus indicum, Pueraria lobata, Pueraria thunbergiana, Robinia pseudoacacia, Sophora japonica, Tamarindus indica, Tephrosia grandiflora, Thermopsis fabacea, Thermopsis montana, Wisteria sinensis, Castanea dentata, Dovyalis caffra, Centaurium littorale, Gentiana lutea, Gentiana macrophylla, Gentiana tibetica, Erodium cicutarium, Geranium maculatum, Geranium sibiricum, Ginkgo biloba, Globularia aphyllanthesa, Globularia trichosanthes, Aesculus hippocastanum, Albuca altissima, Hosta fortunea, Lachenalia pusilla, Philadelphus incanus, Nemophila maculata, Nemophila menziesii, Phacelia bolanderi, Phacelia campanularia, Phacelia tanacetifolia, Belamcanda chinensis, Gladiolus cunonius, Iris germanica, Iris notha, Iris pseudoacorus, Iris sibirica, Iris versicolor, Juglans nigra, Juncus acutus, Acinos thymoides, Agastache mexicana, Agastache nepetoides, Ballota nigra, Calamintha nepeta, Clinopodium vulgare, Elsholtzia stauntonii, Glechoma hederacea, Hyssopus officinalis, Lavandula officinalis, Leonotis nepetifolia, Leonurus cardiaca, Leonurus sibiricus, Lycopus europeaus, Marrubium incanum, Mentha spicata, Monarda citriodora, Monarda fistulosa, Nepeta cataria, Origanum hirtum, Perilla frutescens, Pogostemon patchouli, Prunella vulgaris, Pycnanthemum pycnanthemoides, Rosmarinus officinalis, Salvia apiana, Salvia mellifera, Salvia officinalis, Satureja hortensis, Schizonepeta tenuifolia, Scutellaria altissima, Scutellaria orientalis, Sideritis syriaca, Stachys byzantina, Teucrium chamaedrys, Thymus cretaceus, Thymus vulgaris, Lanaria lanata, Stauntonia hexaphylla, Cinnamomum camphora, Laurus nobilis, Lilium martagon, Limnanthes douglasii, Linum grandiflorum, Linum hirsutum, Linum perenne, Kissenia capensis, Mentzelia lindleyi, Strychnos spinosa, Lagerstroemia indica, L,iriodendron tulipifera, Magnolia grandiflora, Alcea rosea, Althaea broussonetifolia, Althaea cannabina, Altbaea officinalis, Gossypium herbaceum, Hibiscus sabdariffa, Hibiscus trionum, Malacothamnus fasciculatus, Dissotis senegambiensis, Toona serrata, Toona sinensis, Montinia caryophyllaceae, Ficus religiosa, Morus nigra, Morina longifolia, Myrica cerifera, Melaleuca altemifolia, Myrtus communis, Abronia nana, Mirabilis jalapa, Forsythia suspensa, Fraxinus excelsior, Fraxinus pennsylvanica, Ligustrum sinense, Syringa amurensis, Syringa villosa, Syringa vulgaris, Camissonia cheiranthifolia, Camissonia pallida, Clarkia amoena, Clarkia rubicunda, Oenothera lamarckianna, Oenothera speciosa, Argemone mexicana, Papaver bracteatum, Papaver dubium, Papaver orientale, Passiflora edulis, Ceratotheca sesamoides, Dicerocaryum eriocarpum, Harpagophytum zeyheri, Sesamum indicum, Phytolacca americana, Cedrus deodara, Pinus pinea, Plantago asiatica, Plantago erecta, Plantago hirtella, Plantago insularis, Plantago major, Plantago sempervirens, Plantago squalida, Platanus occidentalis, Armeria juniperifolia, Limonium gmeinii, Bouteloua curtipendula, Cortaderia selloana, Cymbopogon flexuosus, Panicum virgatum, Polypogon monspeliensis, Schizachyrium scoparium, Setaria sphacelata, Sorghum sudanense, Vetiveria zizanoides, Podocarpus falcatus, Gilia capitata, Gilia tricolor, Phlox drummondii, Phlox paniculata, Securidaca longepedunculata, Polygonum hydropiper, Polygonum lapathifolium, Polygonum orientale, Polygonum punctatum, Rheum palmatum, Rumex acetosa, Rumex crispus, Calandrinia ciliata, Claytonia perfoliata, Portulacca oleracea, Anagallis arvensis, Primula elatior, Grevillea robusta, Punica granatum, Aconitum napellus, Adonis aestivalis, Aquilegia transsilvanica, Aquilegia vulgaris, Clematis manschurica, Clematis virginiana, Delphinium ajacis, Nigella arvensis, Nigella damascena, Nigella sativa, Thalictrum flavum, Trollius europaeus, Reseda luteola, Reseda odorata, Ceanothus cuneatus, Paliurus spina-christi, Rhamnus cathartica, Agrimonia eupatoria, Aruncus dioicus, Aruncus kamtschaticus, Cercocarpus betuloides. Chaenomeles japonica, Geum faurieri, Geum urbanum, Holodiscus discolor, Pentaphylloides mandschurica, Potentilla recta, Potentilla tormentilla, Prinsepia sinensis, Sanguisorba officinalis, Galium spurium, Gardenia jasminoides, Rubia tinctorum, Phellodendron amurense, Cardiospermum grandiflorum, Cardiospermum halicacabum, Koelreuteria paniculata, Sideroxylon inerme, Anemopsis californica, Bergenia cordifolia, Heuchera pilosissima, Tellima grandiflora, Schisandra chinensis, Antirrhinum majus-maximum, Collinsia canadensis, Hebenstreitia fastigiosa, Linaria vulgaris, Mimulus guttatus, Penstemon barbatus, Scrophularia nodosa, Vemonia noveboracensis, Veronica spicata, Simmondsia chinensis, Atropa bella-donna, Brugmansia suaevolens, Capsicum chinense, Datura stramonium, Hyoscyamus niger, Lycium barbatum, Nicandra physaloides, Physalis ixocarpa, Solanum aculeatissimum, Solanum dulcamara, Withania somnifera, Brachychiton discolor, Firmiana simplex, Fremontodendron californicum, Sterculia africana, Sterculia quinqueloba, Strelitzia nicolae, Strelitzia reginae, Cryptomeria japonica, Cunninghamia lanceolata, Taxodium distichum, Camellia japonica, Camellia sinensis, Grewia biloba, Grewia flavescens, Tropaeolum majus, Celtis occidentalis, Ulmus americana, Urtica dioica, Caryopteris sp., Verbena officinalis, Vitex agnus-castus, Cyphostemma juttae, Vitis amurensis, Vitis labrusca, Welwitschia mirabilis, Elettaria cardarnomum, Larrea tridentata, Peganum harmala, and Tribulus terrestris.

In addition, the following plants responded to acetic acid by yielding extracts that inhibited an anti-cancer activity observed in the extracts of un-elicited plants: Beaucarnea recurvata, Cotinus coggygria, Eryngium campestre, Aristolochia clematis, Cnicus benedictus, Grindelia robusta, Hieracium pilosella, Dianthus barbatus, Saponaria officinalis, Euonymus koopmannii, Luffa aegyptiaca, Dioscorea batatas, Elaeagnus angristifolia, Trigonella foenum-graecum, Psidium guajava, Eschscholzia caespitosa, Consolida oeientalis, Mimulus puniceus, Ailanthus altissima, and Valeriana officinalis.

While the data indicate that some plants do not express detectable anti-cancer activity, and that others show activity in both elicited and non-elicited samples, it is significant that approximately 55% of all active plant species exhibit anti-cancer activity only after being elicited with acetic acid. The data demonstrate the potential of acetic acid to induce biosynthetic pathways resulting in de novo synthesized compound(s) with anti-cancer activity, and/or triggering existing biosynthetic pathways, resulting in accumulation of anti-cancer compounds. Without wishing to be bound by theory, it is noted that while these cytotoxic compounds are probably biosynthesized as defense chemicals for the plant itself, they represent a vast potential in the field of anti-cancer drug development using natural products.

These and other advantages should be apparent to those skilled in the art from the teachings herein. Numerous modifications and variations of the present invention are possible and, therefore, within the scope of the appended claims. Consequently only such limitations as appear in the appended claims should be placed on the invention.

What is claimed is:

1. A method for eliciting a compound having therapeutic activity from a plant or plant part, comprising the steps of:
a) contacting a living, intact plant or plant part with an effective amount of acetic acids; and
b) allowing the acetic acid to induce or improve the production of a compound from the plant or plant part.

2. The method of claim 1, wherein the plant or plant part is contacted with an acetic acid in a concentration of about 0.1% acetic acid.

3. The method of claim 1, wherein the aqueous medium is water.

4. The method of claim 1, further comprising recovering the compound from the plant or plant part.

5. The method of claim 4, wherein the recovery step comprises extracting or exuding the compound into an aqueous medium and collecting the compound from the aqueous medium.

6. The method of claim 5, wherein the extracting comprises macerating the plant or plant parts in an aqueous medium.

7. The method of claim 1, wherein the plant part is a plant root.

8. The method of claim 1, wherein the therapeutic activity is selected from the group consisting of anti-microbial activity and anti-cancer activity.

9. The method of claim 8 wherein the anti-microbial activity is selected from the group consisting of anti-bacterial activity and anti-fungal activity

10. The method of claim 1, further comprising providing a chemical library of compounds recovered from the aqueous medium in an amount sufficient to assay for biological activity.

11. The method of claim 1, wherein the step of extracting the compounds comprises removing cuticular material located on the surface of a leaf by contacting the leaf surface with a solvent.

12. The method of claim 11, wherein the media is a liquid medium or an agar medium.

13. The method of claim 11, wherein the cuticular material is selected from the group consisting of lipid, wax, cutin, protein, primary metabolite and secondary metabolite.

14. The method of claim 13, wherein the cuticular material is a wax.

15. The method of claim 11, wherein the solvent is an organic solvent.

16. The method of claim 15, wherein the organic solvent is selected from the group consisting of methylene chloride and chloroform.

17. The method of claim 11, further comprising assaying the solvent solution for therapeutic activity.

18. The method of claim 17, further comprising analyzing the solvent solution to identify an agent which has the therapeutic activity.

19. The method of claim 17, wherein the therapeutic activity is selected from the group consisting of anti-microbial activity and anti-cancer activity.

20. The method of claim 19, wherein the anti-microbial activity is selected from the group consisting of anti-bacterial activity and anti-cancer activity.

21. The method of claim 17, wherein the step of assaying the solvent solution comprises contacting the solution with a medium containing a living microorganism and determining the rate of growth of the microorganism, whereby an inhibition of the growth of the microorganism is indicative of an agent in the solvent solution having therapeutic activity.

22. The process of claim 1, wherein the plant or plant part is obtained from a plant of a species selected from the group consisting of Atropa Belladonna, Erythrina flabelliformis, Ipomoea tricolor, Erythrina crista, Celosia cristata, Gallium spurium, Laurus nobilis, Vitis Labrusca, Gratiola Officinalis, Symphitum Officinalis, Hosta fortuna, Cassia hebecarpa, Thalictrum flavum, Scutellaria altissima, Portulacca oleracea, Portulacca oleracea, Scutellaria certicola, Physalis creticola, Geum Fauriei, Gentiana tibetica, Linum hirsutum, Aconitum napellus, Aconitum napellus, Podophyllum emodii, Thymus cretaceus, Hosta fortunei, Carlina acaulis, Chamaechrista fasciculata, Pinus Pinea, Pegamun hamalis, Amarindus india, Carica papaya, Cistus incanus, Capparis spinosa inermis, Cupressus lusitanica, Diopiros kaka, Eryngium campestre, Aesculos woerlitzensis, Aesculus Hippocastanum, Cupressus sempervirens, Celtis occidentalis, Polygonum cuspidatum, Elaeagnus angustifolia, Elaeagnus commutata, Gentiana macrophylla, Brassica rapa, Sesbania exaltata, Sesbania speciosa, Spartina potentiflora, Brassica juncea, Helianthus annus, Poinsettia sp., Pelargonium zonale,, Leontopodium alpinum, Lupinus luteus, Buxus microphylla, Liatris spicata, Primula japonica, Betula nigra, Filipendula vulgrais, Lobelia siphilitica, Grevillea robusta, Reseda luteola, Gentiana Littoralis, Campanula carpatica, Aesculus hippocastanum, Aesculus woerlitzensis, Ageratum conizoides, Psidium guajava, Ailanthus altissima, Buxus microphylla “japonica” Hydrocotyle asiatica, Grevillea robusta, Brugmansia suaveolens, Thymus pulegioides, Thymus lema-barona, Thymus serphyllum (wild), Gaultheria procumbens, Thymus serphyllum, Thymus camosus, Thymus thracicus, Calycanthus floridus, Zingiber officinalis, Lamium dulcis, “argenteus”, Thymus praecox “arcticus”, Thyrnus pulegioides “lemons”, Thymus speciosa, Thymus camosus, Thymus pseudolamginosus, Thymus vulgraris “oregano”, Ficus religiosa, Forsythia suspensa, Chelidonium majus, Thymus wooly, Thymus portugalense, Nicotiana tabacum, Thymus cytridorus “aureus”, Cactus officinailis, Lal lab purpurea, Juglans regia, Actinidia chinensis, Hemerocallis, Betula pendula, Gardenia jasminoides, Taxodium distichum, Magnolia loebherii, Crataegus praegophyrum, Larix decidua, Thuja ocidentalis, Thuja orientalis, Cupressocyparis leylandii, Pseudotsuga menziesii, Abies firma, Parthenocissus quinquefolia, Allium cemum (wild), Juniperus blue “pacific” taraxacum officinalis, Yucca sp., Ilexaquifolium, Tsuga canadensis “penola”, Ilex aquifolium, Tsuga canadensis “penola”, Ilex cornuta, Taxus hiksii, Taxus media, Metasequoia glyptostroboides, Pinus bungiana, Buxus sempervirens, Stewartia koreana, Prunus Sp., Betula dahurica, Plantago minor, Acer palmatum “burgundy”, Acer campestre, Cotynus coggygria, Quercus robur “fastigiata”, Acer truncatum, Achyranthes bidentata, Allium japonicum, Carum capsicum, Agastache mexicana, Prunella vulgaris, Tagetes minuta, Prunella vulgaris, Nepeta cataria, Ratibida columnifera-Fera. Aster-Nova anglicae, Myrica cerifera, Pittosporum tobira, Taxodium distichum (H20), Taxodium distichum (Acetic acid), Plantago major, Pinus sylvestris, Acorus canadensis, Pieris Japonica, Pinus strobus, Trifolium pratense, Prunus serotica, Datura stramonium, Geranium maculata, Hydrocotyle asiatica, Taxodium distichum, Astragalus sinicus, Centauria maculata, Ruschia indurata, Myrthus communis, Platanus occidentalis, Licium barbatum, Lavandula officinalis, Grevillea robusta, Hippophae rhamnoides, Filipendula ulmaria, Betula pendula, Polygonum odoratum, Brugmansia graveolens, Rhus toxicodenta, Armoracia rusticana, Ficus benjaminii, Sluffera sp, Pelagonium zonale, Allium sp, Asimina triloba, Lippa dulcis, Epilobium augustifolium, Brugmansia suavecolens (old), Brugmansia suaveolens (young), Xanthosoma sagittifolium. (leaf), Xanthosoma sagittifolium (stem), Monstera deliciosa., Aglaonema commutatus, Dieffenbachia leopoldii, Anthurium andreanum, Syngonium podophyllum, Dracaena fragrans, Ananas comosus, Strelitzia reglinae, Diffenbachia segiunae, Syngonium aurutum, Dracaena sq, haemanthus katharina, Anthurium altersianum, Spathiphyllum grandiflorum, Spathiphyllum. cochlearispatum, Monstera, pertusa, Anthurium magnificum, Anthurium hookeri, Anthurium elegans, Calathea zebrina, Yucca elephantipes, Bromelia balansae, Musa textiles, Myrthus communis, Olea olcaster, Olea europaea, Verium oleander, Cocculus laurifolius, Microsorium punctatum, Ficus sp., Senseviera sp., Adansonia digitata, Boechimeria boloba, Piper nigrum, Phymatosorus scolopendria, Tumera ulmifolia, Nicodemia diversifolia, Tapeinochilos spectabilis, Rauwolfia tetraphylla, Ficus elastica, Cycas cirinalis, Caryota ureus, Cynnamonum zeylonicum, Aechmea luddemoniana, Foenix zeulonica, Ficus benjamina, Ficus pumila, Murraya exotica, Trevesia sungaica, Clerodendrum speciossicum, Actinidi colonicta, Paeonia lactiflora, Paeonia suffructicisa, Quercus imbricaria, Iris alida, Portulacca olleracea, Poligonum aviculare, Iris pseudocarpus, Allium nutans, Allium fistulosum, Antericum ramosum, Veratrum nigrum, Polygonum latifolia, Hosta lancefolia, Hosta zibalda, Echinops sphae, Paeonia dahurica, Inula hilenium, Trambe pontica, Digitalis lutea, Bactisia australis, Austolachia australis, Hissopus zeraucharicus, Feucrium ham. edris., Sedum album, Heraclelum pubescens, Origanum vulgare, Cachris alpina, Haser trilobum, Matteuccia. struthiopteris, Sedum telchium, Bocconia cordata, Ajuga reptans, Thalictrum minus, Anemona japonica, Clematis rectae, Thalictrum sp., Alchemilla sp., Potentilla alba, Poterium sangiusorba, Menispernum dauricum, Oxybaphus nyctagineus. Anroracea rusticana, Crambe cordifolia. A rimonia eupatora, Anchusa officinalis, Poly monium ceruleum, Valeriana officinalis, Pulmonaria molissima, Stachys lanata, Coronilla varia, Platycarya grandiflora, Lavandula officinalis, Vincetoxicum officinale, Acalypha hispida, Gnetum gnemon, Psychotria nigropunctata, Psychotria metbacteriodomasica, Codiaeum variegatum, Phyllanthus grandifolius, Pterigota alata, Pachyra affinis, Sterculia elata, Philodendron speciosum, Pithecellobium unguis-cati, Sanchezia nobilis, Oreopanax capitatus, Ficus triangularis, Kigelia pinnata, Piper cubeba, Laurus nobilis, Erythrina caffra, Metrosideros excelsa, Osmanthus fragrans, Cupressus sempervirens, Jacobinia sp., Senecio platyphylloides, Livistona chinensis, Tetraclinis articulata, Eucalyptus rudis, Podocarpus spinulosus, Eriobotrya japonica, Gingko biloba, Rhododendron, Thuja occidentalis, Fagopyrum suffruticosum, Geum macrophylilum, Magnolia kobus, Vinca minor, Convallaria majalis, Corylus avellana, Berberis sp., Rosa multiflora, Ostrya carpinifolia, Ostrya connogea, Quercus rubra, Liriodendron tulipifera, Sorbus aucuparia, Betula nigra (leaf), Betula nigra (flower), Castanea sativa, Bergenia crassifolia, Artemisia dracunculus, Ruta graveolens, Quercus nigra, Schisandra chinensis, Betula alba, Sambucus nigra, Gentiana cruciata, Encephalartos horridus, Phlebodium aureum, Microlepia platyphylla, Ceratozamia mexicana, Stenochlaena tenuifolia, Adiantum trapeziforme, Adiantum raddianum, Lygodium japonicum, Pessopteris crassifolia, Asplenium australasicum, Agathis robusta, Osmunda regalis, Osmundastrum claytonianum, Phyllitis scolopendrium, Polystichum braunii, Cyrtomium fortunei, Dryopteris filix-mas, Equisetum variegatum, Athyrium nipponicum, Athyrium filix-femina, Parthenocissus tricuspidata, Ligusticum vulgare, Chamaeciparis pisifera, Rosa canina, Cotinus coggygria, Pinus strobus, Celtis occidentalis, Picea schrenkiana, Cydonia oblonga, Ulmus pumila, Euonymus verrucosus, Deutzia scabra, Mespilus germanica, Quercus castaneifolia, Euonymus europea, Securinega suffruticosa, Koelreuteria paniculata, Syringa josikaea, Zelkova carpinifolia, Abies cephalonica, Taxus baccata, Taxus cuspidata, Salix babylonica, Thuja occidentalis, Actinidia colomicta, Mahonia aquifolium, Aralia mandschurica, Juglans nigra, Euonymus elata, Prinsepia sinensis, Forsythia europaea, Sorbocotoneaster pozdnjakovii, Morus alba, Crataegus macrophyllum, Eucommia ulmifolia, Sorbus commixta, Philodendron amurense, Cornus mas, Kerria japonica, Parrotia persica, Jasminum fruticans, Swida sanguinea, Pentaphylloides fruticosa, Sibiraea altaiensis, Cerasus japonica, Kolkwitzia amabilis, Amigdalus nana, Acer mandschurica, Salix tamarisifolia, Amelanchier spicata, Cerasus mahaleb, Prunus cerasifera, Corylus avellana, Acer tataricum, Viburnum opulus, Syringa vulgaris, Fraxinus exelsior, Quercus trojana, Chaenomelis superba, Pinus salinifolia, Berberis vulgaris, Cotoneaster horisontalis, Cotoneaster fangianus, Fagus sylvatica, Pinus pumila, Pinus sylvestris and Berberis thunbergii.

23. A method of preparing a composition having therapeutic activity, comprising the steps of:
(a)contacting a living intact plant or plant part with an effective amount of acetic acid;
(b) allowing the acetic acid to induce a compound or component having therapeutic activity from the plant or plant part; and
(c)collecting a composition comprising the compound or component.

24. The method of claim 23, wherein the composition is collected by macerating the plant or plant parts in an aqueous medium.

25. The method of claim 23, wherein the composition is collected by contacting a surface of the plant or plant parts with a solvent suitable for removing cuticular or epicuticular material.

26. The method of claim 23, wherein the amount of acetic acid is about 0.1% acetic acid.
